(19) United States
(12) Patent Application Publication
(10) Pub. No.: US 2025/0160620 A1
(43) Pub. Date: May 22, 2025

(54) METHODS AND APPARATUSES FOR REDUCING CURVATURE OF A COLON

(71) Applicant: Neptune Medical Inc., Burlingame, CA (US)

(72) Inventors: Francisco G. LOPEZ, San Mateo, CA (US); Neal TANNER, Austin, TX (US); Mark C. SCHEEFF, Oakland, CA (US); Alexander Q. TILSON, Burlingame, CA (US)

(21) Appl. No.: 18/723,414

(22) PCT Filed: Dec. 22, 2022
(86) PCT No.: PCT/US2022/082300

§ 371 Date: Jun. 21, 2024

Related U.S. Application Data
(60) Provisional application No. 63/265,934, filed on Dec. 22, 2021.

(51) Int. Cl. A61B 1/00; G16H 40/63

(52) U.S. Cl. CPC A61B 1/00131; A61B 1/00147; G16H 40/63

(57) ABSTRACT

Described herein are methods and apparatuses for reducing the curvature of a body lumen, such as a colon, using a robotic system. In particular, described herein are methods and apparatuses for reducing curvature of a body lumen (e.g., colon) using apparatuses comprising one or more, including two or more, elongate rigidizable members. The elongate rigidizable members may be arranged telescopically and may be activated by applying positive pressure and/or vacuum.

### CROSS REFERENCE TO RELATED APPLICATIONS

This patent application claims priority to U.S. provisional patent application No. 63/265,934, titled “METHODS AND APPARATUSES FOR REDUCING CURVATURE OF A COLON,” filed on Dec. 22, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

During medical procedures, the interventional medical device can curve or loop through the anatomy, making advancement of the medical device difficult. Gastrointestinal looping, caused when the endoscope can no longer advance due to excessive curving or looping of the gastrointestinal tract, is a particularly well-known clinical challenge for endoscopy. Indeed, one study found that looping occurred in 91 of 100 patients undergoing colonoscopy [Shah et al, “Magnetic Imaging of Colonoscopy: An Audit of Looping, Accuracy and Ancillary maneuvers.” Gastrointest Endosc 2000; 52:1-8]. Gastrointestinal looping prolongs the procedure and can cause pain to the patient because it can stretch the vessel wall and the mesentery. Furthermore, gastrointestinal looping leads to an increased incidence of perforations. In severe cases of gastrointestinal looping, complete colonoscopies are impossible since looping stretches the length of the colon and the colonoscope is not long enough to reach the end. Gastrointestinal looping is an impediment to precise tip control, denying the user the coveted one-to-one motion relationship between the handle and the endoscope tip. Such problems commonly occur across a wide range of endoscopic procedures, including colonoscopy, esophagogastroduodenoscopy (EGD), enteroscopy, endoscopic retrograde cholangiopancreatography (ERCP), interventional endoscopy procedures (including ESD (Endoscopic Submucosal Dissection) and EMR (Endoscopic Mucosal Resection)), robotic flexible endoscopy, trans-oral robotic surgery (TORS), altered anatomy cases (including Roux-en-Y), and during NOTES (Natural Orifice Transluminal Endoscopic Surgery) procedures. Accordingly, there is a need for device that helps prevent gastrointestinal looping to provide more successful access to the gastrointestinal tract.

Similar difficulties in advancing medical instruments can arise, for example, during interventional procedures in the lungs, kidneys, brain, cardiac space, and other anatomical locations. Accordingly, there is a need for a device that can provide safe, efficient, and precise access to otherwise difficult to reach anatomical locations.

### SUMMARY OF THE DISCLOSURE

Described herein are methods and apparatuses for reducing the curvature of a body lumen, such as a colon, using a robotic system. In particular, described herein are methods and apparatuses for reducing curvature of a body lumen (e.g., colon) using apparatuses comprising one or more, including two or more, elongate rigidizable members. The elongate rigidizable members may be arranged telescopically and may be activated by applying positive pressure and/or vacuum.

For example, described herein are method of reducing curvature of a colon using a robotic system, comprising: navigating an elongate member distally into the colon; and activating a set of movements of the robotic system to control a steerable distal end region of the elongate member relative to a wall of the colon while withdrawing a proximal portion of the elongate member proximally out of the colon to reduce the curvature of the colon. Any of these methods may include maintaining the reduction of the curvature of the colon while performing one or more actions using the elongate member.

In any of the methods described herein activating the set of movements of the robotic system may comprise adjusting the rigidity of the elongate member. For example, the method may include making the elongate member (e.g., an inner rigidizing member nested within an outer rigidizing member) flexible. In any of these examples activating the set of movements of the robotic system may comprise activating a set of preprogrammed movements. Any of these methods may include activating the set of movements of the robotic system by withdrawing the proximal portion of the elongate member proximally out of the colon while maintaining a position and/or configuration of a distal end region of the elongate member. The position and/or configuration may be maintained by forming and maintaining a hook using a steerable distal end region. In some examples the position and/or configuration may be maintained by attaching a distal end region to the wall of the colon. In some examples, the position and/or configuration may be maintained by steering the steerable distal end region to maintain the lumen-centricity of the distal end region within the colon.

For example, described herein are methods of reducing curvature of a colon using a robotic system, the method comprising: navigating an elongate member distally into the colon; activating a set of movements of the robotic system to engage a wall of the colon with the elongate member to reduce the curvature of the colon; and maintaining the reduction of the curvature of the colon while performing one or more actions using the elongate member.

Activating the set of movements of the robotic system may comprise activating a set of preprogrammed movements (e.g., a macro). The elongate member may comprise a first elongate member and a second elongate member that is axially slideable within the first elongate member, further wherein activating the set of movements of the robotic system comprises allowing at least one of the first elongate member and the second elongate member to freely rotate at a proximal end region of the elongate member that is outside of the colon. In any of these examples, the first elongate member and the second elongate member may be configured to freely rotate, to be driven in rotation, and/or to operate in admittance rotation control, or some combination of any of these. The elongate member may comprise a first elongate member and a second elongate member, wherein the second elongate member is axially slideable within the first elongate member, further wherein activating the set of movements of the robotic system comprises actively rotating either or both the first elongate member and the second elongate member at a proximal end region of the elongate member that is outside of the colon. In some examples actively rotating either or both the first elongate member and the second elongate member comprises actively rotating the first elongate member or the second elongate member in a direction in which the first elongate member or the second elongate member has a different bending stiffness in different rotational directions. Activating the set of movements of the robotic system may comprise activating a set of preprogrammed movements. For example, activating the set of movements of the robotic system may comprise forming a hook or bend at a distal end of the elongate member and maintaining an angle between the hook or bend and the colon while changing an articulation plane of the elongate member proximal to the hook or bend. The hook or bend may engage with the distal tissue region. Alternatively or additionally the distal end of the elongate member (e.g., elongate member) may couple via an expandable member (e.g., balloon, expandable frame, etc.) and/or by suction.

In some examples activating the set of movements of the robotic system comprises at least partially rigidizing the elongate member. The elongate member may comprise a first elongate member and a second elongate member that is axially slideable within the first elongate member, further wherein activating the set of movements of the robotic system comprises partially rigidizing ether the first elongate member or the second elongate member. Partial rigidization may allow the user to adjust (in some cases dynamically adjust) the rigidity of one or more elongate members (e.g., robotic members) which may provide a safer technique to reduce the curvature of the colon and/or to reduce the curvature of the elongate member(s). For example, partial rigidization may limit the force between the elongate member(s) and the colon when manipulating the elongate members.

Activating the set of movements of the robotic system may comprise forming a curve or hook at a distal end region of the elongate member while changing an articulation plane of the elongate member proximal to the distal end region. For example, the distal end region of the elongate member may be secured relative to the wall by one or more of: an inflatable balloon or suction. For example, a vacuum chuck may be included. In some examples activating the set of movements of the robotic system to engage a wall of the colon comprises forming a pleat in the colon by pulling the wall of the colon proximally using the elongate member.

Any of these methods may include determining a shape of the elongate member within the colon. Determining a shape of the elongate member may comprise sensing the shape of the elongate member. In some examples determining a shape of the elongate member comprises estimating the shape of the elongate member based on a history of the individual articulation of the elongate member within the colon.

Any of these methods may include visualizing the colon using one or more imaging sensors on the elongate member. Activating the set of movements of the robotic system may comprise using a machine learning agent to guide the set of movements. Movements may include axial/distal movement (e.g., advancing/retracting) and/or rotational movement (clockwise/counterclockwise) and/or steering the elongate member (e.g., in one example, steering the distal end region of one or more elongate members, as by pullwires). Movements may be done automatically or manually or manually with automatic assistance.

The elongate member may comprise a first elongate member and a second elongate member that is axially slideable within the first elongate member, further wherein navigating an elongate member distally into the colon comprises alternately rigidizing and un-rigidizing the first elongate member and the second elongate member. The one or more actions may comprise one or more of: visualizing the wall of the colon; removing tissue from the wall of the colon, treating the wall of the colon; applying energy to the wall of the colon; applying a material to the wall of the colon.

For example, a method of reducing curvature of a colon using a robotic system may include: inserting a elongate member into the colon, the elongate member comprising a first elongate member and a second elongate member, wherein the second elongate member is axially slideable within the first elongate member; advancing the elongate member through a lumen of the colon; maintaining a position of a distal end of the elongate member relative to the lumen of the colon; and controlling the elongate member to reduce the curvature in the colon.

Controlling the elongate member to reduce the curvature in the colon may include automatically articulating the distal end of the first or second elongate member of the robotic system to maintain a hook or curve shape in a plane distal relative to the colon. Automatically rotating the distal end of the first or second elongate member to maintain the distal end at or near a center of the colon may include using gathered image or shape-sensing data. Maintaining a position of a distal end of the elongate member may include controlling the robotic system to inflate a balloon to anchor the first or second elongate member to the colon.

Maintaining a position of a distal end of the elongate member may include controlling the robotic system to apply vacuum to anchor the first or second elongate member to the colon. Advancing the elongate member through the colon may comprise: inserting the first elongate member into the colon while the first elongate member is in a flexible configuration; supplying vacuum or pressure to the first elongate member to transition the first elongate member into a rigid configuration; inserting a second elongate member in a flexible configuration through the first elongate member while the first elongate member is in the rigid configuration such that the second elongate member takes on a shape of the first elongate member in the rigid configuration; and supplying vacuum or pressure to the second elongate member to transition the second elongate member from the flexible configuration to a rigid configuration. Any of these methods may include controlling the robotic system to transition the first elongate member and the second elongate member to the flexible configurations prior to reducing the curvature in the colon.

The methods described herein may include controlling the robotic system to drive rotation of the first elongate member. In some examples the methods described herein may include anchoring the second elongate member during rotation of the first elongate member. Any of these methods may include driving rotation of the second elongate member. For example, any of these methods may include anchoring the first elongate member during rotation of the second elongate member.

Controlling the elongate member to reduce the curvature in the colon may include controlling the robotic system to retract the first elongate member while allowing it to freely rotate relative to a handle at its proximal end. For example, controlling the elongate member to reduce the curvature in the colon comprises controlling the robotic system to retract the second elongate member while allowing it to freely rotate relative to a handle at its proximal end. Controlling the elongate member to reduce the curvature in the colon may include automatically rotating the first elongate member to a desired rotation.

Automatically rotating the first elongate member to a desired rotation may include rotating the first elongate member until gathered data confirms that the first elongate member is moving. For example, automatically rotating the first elongate member to a desired rotation may comprise the robotic system stopping rotation if gathered torque data indicates that applied torque exceeds a threshold level. Controlling the robotic system may include rotating to a rotational position corresponding to a more flexible axis of the first elongate member than surrounding portions of the first elongate member. For example, automatically rotating the first elongate member to a desired rotation may result in the first elongate member bending along its more flexible axis.

Any of these methods may include allowing the first elongate member to freely rotate within a handle at its proximal end. Maintaining the position of the distal end of the elongate member relative to the lumen of the colon may comprise controlling the robotic system to deflect a distal end of the second elongate member such that it hooks onto a curve in the colon and further comprising the robotic system automatically rotating the distal end of the first or second elongate member to maintain the distal end at or near a center of the colon.

Any of these methods may include sensing a shape of the colon and/or sensing an amount of rotational torque applied to a proximal end of the first elongate member and/or a proximal end of the second elongate member.

Any of these methods may include receiving visual feedback corresponding to a position of the first and/or second elongate member. For example, the method may include automatically adjusting or maintaining a position of the first and/or second elongate member based on the visual feedback, and/or prompting a physician to adjust a position of the first and/or second elongate member based on the visual feedback.

In any of these methods, controlling the elongate member to reduce the curvature in the colon may include controlling the robotic system to rotate a distal end of the second elongate member while the distal end is fixed to the colon while allowing at least one of the first and second elongate member to freely rotate at their proximal ends. Controlling the elongate member to reduce the curvature in the colon may comprise controlling the robotic system to rotate the first elongate member in a first direction and the second elongate member in a second direction, opposite to the first direction. In some examples controlling the elongate member to reduce the curvature in the colon comprises advancing the first and second elongate members to a curve in the colon before fixing the first or second elongate member to the colon; and controlling the robotic system to retract the first and second elongate member such that at least a portion of the colon proximal to a distal end of the first and second ends folds together, reducing the length of the portion is reduced as the first and second elongate member are retracted. Fixing the first or second elongate member to the colon may comprises controlling the robotic system to apply suction to fix a distal end of the first elongate member to the colon. For example, fixing the first or second elongate member to the colon may comprise controlling the robotic system to inflate a balloon on the first elongate member to anchor a distal end of the first elongate member to the colon.

Also described herein are systems for performing any of these methods. For example, a system may include: an elongate member configured to be inserted to a colon; a base coupled to a proximal end of the elongate member; a controller comprising one or more processors; and a memory coupled to the one or more processors, the memory storing computer-program instructions, that, when executed by the one or more processors, perform a computer-implemented method to reduce a curvature of the colon, the method comprising: activate a set of movements of the robotic system to engage a wall of the colon with the elongate member to reduce the curvature of the colon; and maintain the reduction of the curvature of the colon while performing one or more actions using the elongate member.

The elongate member may include a first elongate member and a second elongate member that is axially slideable within the first elongate member. The elongate member may be rigidizable. The elongate member may comprise a first rigidizable elongate member and a second rigidizable elongate member that is axially slideable within the first elongate member.

Activating the set of movements of the robotic system may comprise allowing at least one of the first elongate member and the second elongate member to freely rotate at a proximal end region of the elongate member that is outside of the colon. Activating the set of movements of the robotic system may include actively rotating either or both the first elongate member and the second elongate member at a proximal end region of the elongate member that is outside of the colon. In some examples actively rotating either or both the first elongate member and the second elongate member may comprise actively rotating the first elongate member or the second elongate member in a direction in which the first elongate member or the second elongate member has a greater bending stiffness relative to other regions of the first elongate member or the second elongate member. Activating the set of movements of the robotic system may comprise activating a set of preprogrammed movements. Activating the set of movements of the robotic system may comprise forming a hook or bend at a distal end of the elongate member and maintaining an angle between the hook or bend and the colon while changing an articulation plane of the elongate member proximal to the hook or bend. In some examples activating the set of movements of the robotic system comprises at least partially rigidizing the elongate member. Activating the set of movements of the robotic system may comprise partially rigidizing ether the first elongate member or the second elongate member. In some examples activating the set of movements of the robotic system comprises securing a distal end region of the elongate member relative to the wall of the colon while changing an articulation plane of the elongate member proximal to the distal end region.

The distal end region of the elongate member may be secured relative to the wall by one or more of: an inflatable balloon or suction.

In some examples activating the set of movements of the robotic system to engage a wall of the colon comprises forming a pleat in the colon by pulling the wall of the colon proximally using the elongate member.

Any of these systems may be configured to determine a shape of the elongate member within the colon. For example, determining a shape of the elongate member may comprise sensing the shape of the elongate member. Determining a shape of the elongate member may comprise estimating the shape of the elongate member based on a history of the individual articulation of the elongate member within the colon.

Any of these systems may include one or more imaging sensors on the elongate member for visualization within the colon.

Activating the set of movements of the robotic system may comprise using a machine learning agent to guide the set of movements. For example, the system may be trained to perform or suggest the user perform a particular set of movements when the system that are likely to result in a reduction of the curvature of the colon based on the training of the machine learning agent operating in the system.

For example, a system may include: a elongate member configured to be inserted to a colon, comprising a first elongate member and a second elongate member that is axially slideable within the first elongate member; a base coupled to a proximal end of the first elongate member and/or the second elongate member; a controller comprising one or more processors; and memory coupled to the one or more processors, the memory storing computer-program instructions, that, when executed by the one or more processors, perform a computer-implemented method to reduce a curvature of the colon, the method comprising: navigate a elongate member distally into the colon; activate a set of movements of the robotic system to engage a wall of the colon with the elongate member to reduce a curvature of the colon; and maintain the reduction of the curvature of the colon while performing one or more actions using the elongate member.

As mentioned, described herein are methods and apparatuses for reducing the curvature of a colon by partially rigidizing the elongate member (e.g., an elongate rigidizable member). For example, described herein are methods of reducing curvature of a colon that include: navigating an elongate rigidizable member in an un-rigidized configuration distally into the colon and around one or more bends within the colon; applying a positive or negative pressure at a first magnitude to rigidize the elongate rigidizable member within the colon to a first rigidity; and withdrawing the elongate rigidizing member proximally out of the colon while adjusting the rigidity of the elongate rigidizing member by adjusting the positive or negative pressure to one or more intermediate magnitudes that is less than the first magnitude but greater than a magnitude of the un-rigidized configuration, so that the one or more bends are reduced.

Any of these methods may include displaying a shape of the elongate rigidizable member, and/or a force on the elongate rigidizable member while withdrawing the elongate rigidizing member proximally. The force may include a torque acting on the elongate rigidizable member and/or an engagement force between the elongate rigidizable member and the wall of the colon.

The magnitude of the positive or negative pressure is typically the absolute value of this positive or negative pressure. The intermediate pressure(s) may be a pressure that is a difference from the prior (e.g., the first) magnitude, so that the change in rigidity is gradually reduced (or in some cases increased) when withdrawing the elongate rigidizable member. For example, the intermediate magnitude may be different by a small percentage from the first magnitude (e.g., 2%, 3%, 4%, 5%, 6%, 7%, 8%, 9%, 10%, etc. lower than the first magnitude). The step of adjusting the rigidity may be performed iteratively until the force acting on the elongate rigidizable member is below the threshold, which may be measured or detected by feel by the user.

In general, the rigidity may be manually, automatically or semi-automatically adjusted. For example the user may manually adjust the rigidity as the user withdraws the elongate rigidizing member proximally. The apparatus (e.g., system) may display the force (s0 acting on the elongate rigidizable member and/or the shape of the elongate rigidizable member, and the user may monitor them and/or the user may base the adjustment on the feel of the retraction, or some combination of these. For example, any of these methods may include adjusting the rigidity of the elongate rigidizing member as the elongate rigidizing member is withdrawn proximally so that one or more forces acting on the elongate rigidizing member do not exceed a threshold.

The one or more forces may comprise a torque force and/or an engagement force between the elongate rigidizing member and a wall of the colon. Adjusting may include automatically adjusting the rigidity of the elongate rigidizing member. Navigating the elongate rigidizing member may comprise telescoping the elongate rigidizing member out of a second elongate rigidizing member. The rigidity of the second elongate rigidizing member may also be adjusted while withdrawing the elongate rigidizing member proximally.

Any of these methods may include maintaining the reduction of the curvature of the colon while performing one or more actions using the elongate rigidizing member. For example, the elongate rigidizable member may be withdrawn partially proximally, with the distal region remaining within the colon to hold the colon in the reduced configuration. The elongate rigidizable member maybe rigidized in this configuration. A second elongate rigidizable member (e.g., an inner elongate rigidizable member) may be advanced through the elongate rigidizable member (the “first” or outer elongate rigidizable member) and steered through the reduced anatomy distally. The second elongate rigidizable member may then be held in position and in some examples rigidized, and (optionally) the first elongate rigidizable member may again be advanced distally over the second elongate rigidizable member. Or one or more procedures may then be performed on the colon wall (e.g., imaging, biopsy, tissue ablation, tissue removal, etc.) through the first (or optionally the second) elongate rigidizable member.

Also described herein are methods and apparatuses for transmitting torque in an elongate rigidizable member, particularly elongate rigidizable members that are formed of a plurality of layers, including a braid layer, that slide over each other in an un-rigidized configuration resulting in poor torque transmission in the un-rigidized configuration. For example, a rigidizing device may include: an elongate flexible tube; a braid layer positioned over the elongate flexible tube; an outer layer over the elongate flexible tube and the braid layer; an inlet between the elongate flexible tube and the outer layer and configured to attach to a source of vacuum or pressure; a sealed channel between the elongate flexible tube and the outer layer, the sealed channel comprising a working channel, a cable guide, or an inflation lumen, wherein the sealed channel is formed at least in part by a torque-transmitting member coupled at a proximal end of the device to a handle region so that torque applied to the handle region is transmitted to a distal end region of the elongate flexible tube; and wherein the rigidizing device is configured to have a rigid configuration when vacuum or pressure is applied through the inlet and a flexible configuration when vacuum or pressure is not applied through the inlet. The torque transmitting member may comprise a laser cut hypotube forming the sealed channel. In some examples, the torque transmitting member comprises a braided member having a wide, torque-transmitting braid angle (e.g., braid angle of greater than 60 degrees, greater than 65 degrees, etc.). The sealed channel may be sealed from the vacuum or pressure applied through the inlet. The braid layer of the rigidizable member may include a plurality of strands braided together at a braid angle of 5-40 degrees relative to a longitudinal axis of the elongate flexible tube when the elongate flexible tube is straight.

A rigidizing device may include: an elongate flexible tube comprising a torque-transmitting layer and a sealing layer covering the torque transmitting layer; a braid layer positioned over the elongate flexible tube; a slip layer adjacent to the braid layer; an outer layer over the elongate flexible tube and the braid layer; and an inlet between the elongate flexible tube and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet; wherein the slip layer is configured to reduce friction between the braid layer and the elongate flexible tube or the outer layer when the rigidizing device is in the flexible configuration, further wherein the torque-transmitting layer is coupled at a proximal end of the device to a handle region so that torque applied to the handle region is transmitted to a distal end region of the elongate flexible tube when the rigidizing device is in the flexible configuration. The torque transmitting layer may be coupled to the elongate flexible tube at a distal end region and/or along its length, in addition to being attached at the proximal handle region. The torque-transmitting layer may comprise a laser-cut hypotube. Any appropriate laser-cut pattern that is highly flexible and that efficiently transmits torque may be used. For example, a pattern of circumferential laser-cut kerfs that provide a high degree of flexibility without sacrificing torqueability may be used.

A rigidizing device may include: an elongate flexible tube; a braid layer positioned over the elongate flexible tube; a slip layer adjacent to the braid layer; an outer layer over the elongate flexible tube and the braid layer, wherein the outer layer comprises a torque-transmitting layer and a sealing layer covering the torque transmitting layer; and an inlet between the elongate flexible tube and the outer layer and configured to attach to a source of vacuum or pressure; wherein the rigidizing device is configured to have a rigid configuration when vacuum or pressure is applied through the inlet and a flexible configuration when vacuum or pressure is not applied through the inlet; wherein the slip layer is configured to reduce friction between the braid layer and the elongate flexible tube or the outer layer when the rigidizing device is in the flexible configuration, further wherein the torque-transmitting layer is coupled at a proximal end of the device to a handle region so that torque applied to the handle region is transmitted to a distal end region of the elongate flexible tube when the rigidizing device is in the flexible configuration. The torque-transmitting layer may comprise a laser-cut hypotube.

The methods and apparatuses described herein may include aspects of, or may be used with the apparatuses, and in particular the dynamically rigidizing methods and apparatuses described, for example in International Application No. PCT/US2020/013937, filed on Jan. 16, 2020, and titled “DYNAMICALLY RIGIDIZING COMPOSITE MEDICAL STRUCTURES,” the entirety of which is incorporated by reference herein.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a rigidizing device.

FIGS. 2A-2B show exemplary rigidized shapes of a rigidizing device.

FIGS. 3A-3B show an example of a portion of a vacuum rigidizing apparatus as described herein. FIG. 3A shows a section through the exemplary vacuum rigidizing member of the apparatus. FIG. 3B shows an enlarged view of a portion of the section, illustrating the arrangement of layers in the un-rigidized configuration.

FIGS. 3C-3F show an example of a portion of a vacuum rigidizing apparatus having multiple braid layers as described herein. FIG. 3C shows a perspective view of the vacuum rigidizing member with the outer layer removed (showing the outermost braid layer). FIG. 3D is an enlarged view of a portion of FIG. 3C. FIG. 3E shows a longitudinal section though the vacuum rigidizing member of FIG. 3C. FIG. 3F is a cross-section through the rigidizing member of FIG. 3C.

FIGS. 4A-4B show an exemplary pressure rigidizing device.

FIG. 5 shows a rigidizing device with a distal end section.

FIG. 6 shows a rigidizing device with a distal end section having a plurality of actively controlled linkages.

FIG. 7 shows a nested rigidizing system.

FIG. 8 shows a nested rigidizing system with a cover between the inner and outer rigidizing devices.

FIGS. 9A-9B show a nested rigidizing system where the outer rigidizing device includes steering and imaging.

FIGS. 10A-10H show exemplary use of a nested rigidizing system.

FIGS. 11A-11D show a robotically controlled rigidizing system.

FIGS. 12A-12B show mechanisms of actuating a robotically controlled rigidizing system.

FIG. 13 shows a drive unit for a robotically controlled rigidizing system.

FIG. 14 shows a slide for use with a robotically controlled rigidizing system.

FIGS. 15A-15B show a robotically controlled rigidizing system.

FIG. 16 shows a pivoting arm for a robotically controlled rigidizing system.

FIG. 17 shows an exemplary user-activated control for a robotically controlled rigidizing system.

FIG. 18A shows a rigidizing system in the flexible configuration sagging.

FIG. 18B shows a rigidizing system with drive wheels that prevent sagging.

FIGS. 19A-19C show a twisting maneuver in the colon.

FIGS. 20A and 20B show an example of a reduction maneuver in the model of a colon.

FIG. 21A schematically illustrates an example of an elongate rigidizable member including an anchoring balloon at the distal end region of the elongate rigidizable member.

FIG. 21B schematically illustrates an example of an elongate rigidizable member including an anchoring suction port at the distal end region of the elongate rigidizable member.

FIGS. 21C-21F illustrate one example of a suction port that may be used as part of the methods and apparatuses described herein.

FIGS. 22A-22D illustrate one example of a proximal end of an elongate rigidizable member configured to allow the elongate rigidizable member rotate relative to a handle driver.

FIGS. 22E and 22F show an example of a handle driver securing an elongate rigidizable member so that it may rotate.

FIGS. 23A-23G illustrate an example of a method of reducing a curvature (e.g., loop) using an elongate rigidizable member that rotates at the proximal end.

FIGS. 24A-24C show an example of a robotic system elongate member articulating and rotating at its distal end.

FIGS. 25A-25C show an example of a robotic system including a pair of telescoping elongate rigidizing members performing a pleating reduction.

FIGS. 26A-26B show an example of an elongate rigidizing member being drawn proximally when highly rigidized within a colon model.

FIGS. 27A-27B show an example of an elongate rigidizing member being drawn proximally when unrigidized.

FIGS. 28A-28C show an example of reducing a model colon using an elongate rigidizing member that is configured to be partially rigidized.

FIG. 29 illustrates one example of a vacuum rigidizing elongate apparatus including a torsionally stiff member.

FIG. 30A illustrates another example of an elongate rigidizable member that includes a torsional stiffening member.

FIG. 30B show an example of a laser-cut hypotube that may be used as part of a torsional stiffening member as described herein.

FIG. 31 schematically illustrates an example of a method of reducing a loop of a colon as described herein.

FIG. 32 schematically illustrates an example of a method of reducing a loop of a colon as described herein.

FIG. 33 schematically illustrates an example of a method of reducing a loop of a colon as described herein.

### DETAILED DESCRIPTION

In general, described herein are methods and apparatuses (devices and systems, including software) for controlling the movement of one or more, and preferably a pair of nested, rigidizing elongate members. These methods and apparatuses may be configured to aid in transporting a scope (e.g., endoscope) or other medical instrument through a curved or looped portion of the body, e.g., a portion of the gastrointestinal tract, including, but not limited to the colon, as well as methods of using them. In particular, described herein are methods and apparatuses for reducing the curvature of a body region, such as a colon. These procedures may be referred to herein as “reductions” and may reduce or undue looping that may otherwise occur when scopes such as endoscopes are inserted and tracked within the body. The method and apparatuses described herein may simplify, automate (or partially automate) and maintain reductions in a manner that may be particularly beneficial when navigating otherwise tortuous body regions. These methods provide improvements and techniques not possible in conventional endoscopy.

The rigidizing elongate members described herein, also referred to herein as rigidizing devices, can be long, thin, and hollow and can transition quickly from a flexible configuration (i.e., one that is relaxed, limp, or floppy) to a rigid configuration (i.e., one that is stiff and/or holds the shape it is in when it is rigidized). In some examples the rigidizing apparatus may include a plurality of layers (e.g., coiled or reinforced layers, slip layers, braided layers, bladder layers and/or sealing sheaths) can together form the wall of the rigidizing devices, which may be referred to as “layered rigidizing apparatuses.” Unless the context makes clear otherwise, the methods and apparatuses described herein may refer to any appropriate rigidizing device, including layered rigidizing apparatuses. For example, the rigidizing devices (members, apparatuses, etc.) described herein may be rigidized by jamming particles, by phase change, by interlocking components (e.g., cables with discs or cones, etc.) or any other rigidizing mechanism. The rigidizing devices can transition from the flexible configuration to the rigid configuration, for example, by applying a vacuum or pressure to the wall of the rigidizing device or within the wall of the rigidizing device. In some examples, with the vacuum or pressure removed, the layers can easily shear or move relative to each other, and with the vacuum or pressure applied, the layers can transition to a condition in which they exhibit substantially enhanced ability to resist shear, movement, bending, torque and buckling, thereby providing system rigidization.

Although the examples of rigidizing apparatuses described herein use pressure (positive pressure and/or negative pressure) to selectively and controllable rigidize, any appropriate rigidizing apparatus may be adapted to perform reductions as described herein.

The rigidizing (e.g., selectively rigidizing) apparatuses described herein can provide rigidization for a variety of medical applications, including catheters, sheaths, scopes (e.g., endoscopes), wires, overtubes, trocars or laparoscopic instruments. The rigidizing devices can function as a separate add-on device or can be integrated into the body of catheters, sheaths, scopes, wires, or laparoscopic instruments. The devices described herein can also provide rigidization for non-medical structures.

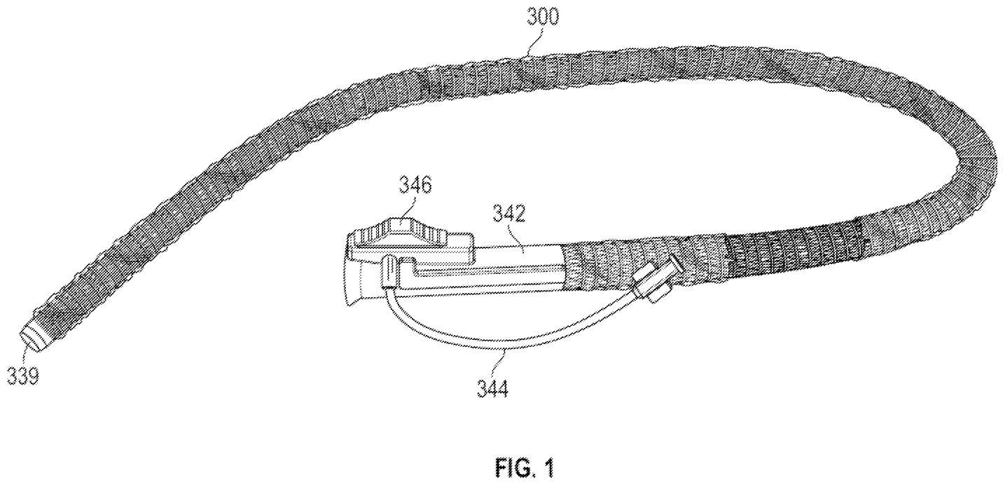

An exemplary rigidizing apparatus is shown in FIG. 1. The system shown includes a rigidizing device 300 having a wall with a plurality of layers including a braid layer, an outer layer (part of which is cut away in this example to show the braid thereunder), and an inner layer. The system further includes a handle 342 having a vacuum or pressure inlet 344 to supply vacuum or pressure to the rigidizing device 300. An actuation element 346 can be used to turn the vacuum or pressure on and off to thereby transition the rigidizing device 300 between flexible and rigid configurations. The distal tip 339 of the rigidizing device 300 can be smooth, flexible, and atraumatic to facilitate distal movement of the rigidizing device 300 through the body. Further, the tip 339 can taper from the distal end to the proximal end to further facilitate distal movement of the rigidizing device 300 through the body. In this example, the rigidizing apparatus is configured as an overtube, but other configurations may be used.

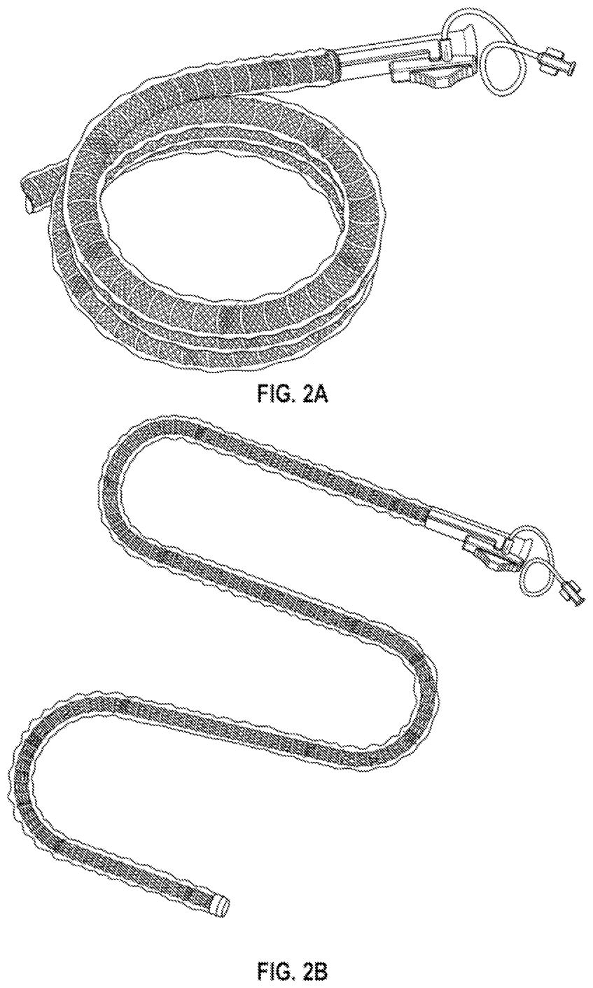

Exemplary rigidizing devices in a rigidized configuration are shown in FIGS. 2A and 2B. As the rigidizing device is rigidized, it locks into the shape it was in before vacuum or pressure was applied, i.e., it does not straighten, bend, or otherwise substantially modify its shape (e.g., it may stiffen in a looped configuration as shown in FIG. 2A or in a serpentine shape as shown in FIG. 2B). The air stiffening effect on the inner or outer layers (e.g., made of coil-wound tube) can be a small percentage (e.g., 5%) of the maximum load capability of the rigidizing device in bending, thereby allowing the rigidizing device to resist straightening. Upon release of the vacuum or pressure, braids or strands within the layers forming the device can unlock relative to one another and again move so as to allow bending of the rigidizing device. Again, as the rigidizing device is made more flexible through the release of vacuum or pressure, it does so in the shape it was in before the vacuum or pressure was released, i.e., it does not straighten, bend, or otherwise substantially modify its shape. Thus, the rigidizing devices described herein can transition from a flexible, less-stiff configuration to a rigid configuration of higher stiffness by restricting the motion between the strands of braid (e.g., by applying vacuum or pressure).

The rigidizing apparatuses described herein can toggle between a rigid configuration and a flexible configuration quickly, and in some examples with an indefinite number of transition cycles. In some examples the degree of rigidization (e.g., the stiffness) of the apparatus may also be adjusted, for example, by adjusting the positive pressure (in examples that are rigidized by positive pressure) or vacuum (in examples rigidized by vacuum). As interventional medical devices are made longer and inserted deeper into the human body, and as they are expected to do more exacting therapeutic procedures, there is an increased need for precision and control. Selectively rigidizing devices (including selectively rigidizing overtubes) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein.

The rigidizing devices described herein can additionally or alternatively include any of the features described with respect to International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, and International Patent Application No. PCT/US2020/013937 filed on Jan. 16, 2020, titled “DYNAMICALLY RIGIDIZING COMPOSITE MEDICAL STRUCTURES,” the entireties of which are incorporated by reference herein.

The rigidizing devices described herein can be provided in multiple configurations, including different lengths and diameters. In some examples, the rigidizing devices can include working channels (for instance, for allowing the passage of typical endoscopic tools within the body of the rigidizing device), balloons, nested elements, and/or side-loading features.

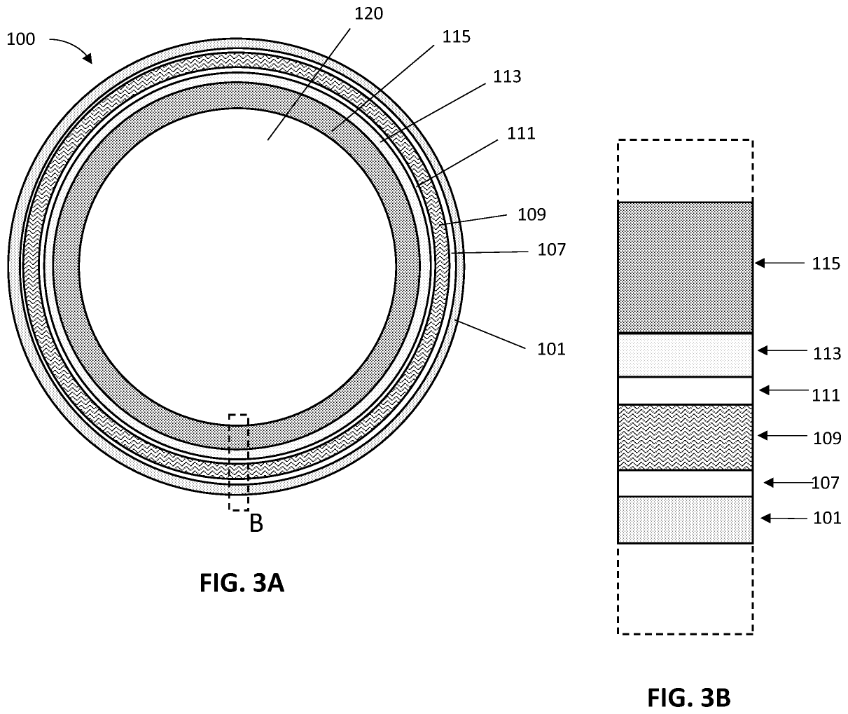

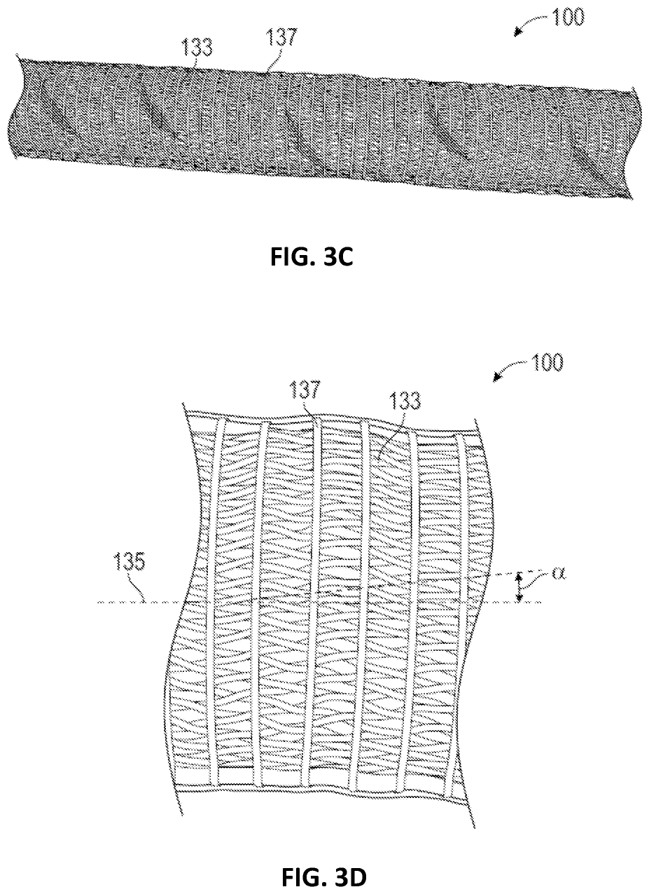

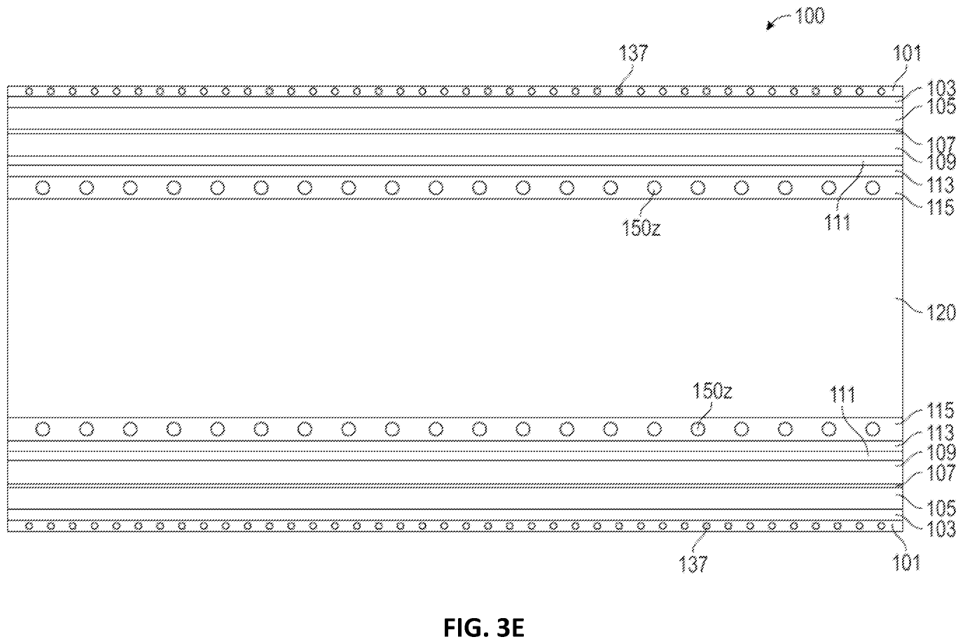

For example, a rigidizing apparatus 100 (also referred to as an apparatus, e.g., system and/or device, including a rigidizable member) may be configured to be rigidized by the application of vacuum, e.g., negative pressure. These apparatuses may generally be formed of layers that are configured to form a laminates structure when negative pressure is applied, so that one or more braided or woven layers may be reversibly fused to a flexible outer layer that is driven against a more rigid inner layer. FIGS. 3A-3B illustrate one example of a section through a rigidizing member of an apparatus (e.g., device, system) that is rigidized by the application of vacuum. FIG. 3B shows an enlarged view of the arrangement of the layers of FIG. 3A in the un-rigidized configuration. In this example, the rigidizable member includes an innermost layer 115 that is configured to provide an inner surface against which the remaining layers can be consolidated (e.g., when vacuum is applied). The innermost layer 115 can include a reinforcement element or coil. The rigidizing member may also include an optional slip layer 113 over (e.g., radially outwards of) the innermost layer. The slip layer may be, e.g., a lubrication, coating and/or powder (e.g., talcum powder) on the outer surface of the inner layer 115 and/or within the gap layer 111. A radial gap layer 111 may separate the inner layer and/or slip layer 113 from a variable stiffness layer that in some examples is a braid, knit and/or woven layer 109 (referred to herein for convenience as a “braid layer”), providing a space between the braid layer and slip layer and/or inner layer for the braided layer(s) to move within, e.g., when no pressure is applied; this space or gap may be removed when pressure is applied, allowing the variable stiffness layer to move radially inward upon application of pressure (e.g., vacuum). A second gap layer 107 may be present between the variable stiffness layer 109 and may be similar to layer 111. As will be described in reference to FIGS. 3C-3F, multiple variable stiffness layers (e.g., braid layers) may be included (e.g., 2, 3 4 or more braid layers may be included) and may be separated by additional gap layers and/or slip layers. The outermost layer 101 can be separated from the variable stiffness layer(s) by a gap layer and in some examples can be configured to move radially inward when a vacuum is applied to pull down against the braid layer(s) and conform onto the surface(s) thereof. In such examples the outermost layer 101 can be soft and atraumatic and can be sealed at both ends to create a vacuum-tight chamber with the innermost layer 115. In some examples the outermost layer may be referred to as a bladder or bladder layer. The outermost layer 101 can be elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK. In some examples a separate bladder layer may be included between the outermost layer and the variable stiffness layer, so that the outermost layer remains relatively smooth while the separate bladder layer is locked onto the variable stiffness layer, stiffening it, with the application of pressure, e.g., negative pressure between the bladder layer and the inner layer or in some examples positive pressure between the outer layer and the bladder layer.

FIGS. 3C-3F illustrate an example of a tubular rigidizing member of an apparatus 100 that includes multiple braid layers. As in FIGS. 3A-3B, the apparatus includes a tube having a wall formed of a plurality of layers positioned around a lumen 120 (e.g., for placement of an instrument or endoscope therethrough). In some examples a vacuum can be supplied between the layers to rigidize the rigidizing device 100. Any of the tubular apparatuses described herein may instead include a solid core forming the inner layer 115.

The innermost layer 115 can be configured to provide an inner surface against which the remaining layers can be consolidated, for example, when a vacuum is applied within the walls of the rigidizing device 100. The structure can be configured to minimize bend force and/or maximize flexibility in the non-vacuum condition. In some examples, the innermost layer 115 can include a reinforcement element 150z or coil within a matrix, as described above. In the example shown in FIG. 3E, the layer 113 over (i.e., radially outwards of) the innermost layer 115 can be a slip layer. The layer 111 can be a radial gap (i.e., a space). The gap layer 111 can provide space for the braided layer(s) thereover to move within (when no vacuum is applied) as well as space within which the braided or woven layers can move radially inward (upon application of vacuum).

As mentioned, the variable stiffness layer 109 includes a plurality of strand lengths that cross each other. The strands lengths can be parts of a single strand (fiber, wire, filament, bundle of filaments, etc.) and/or parts of separate strands. In some examples the variable stiffness layer is configured as a first braid layer including braided strands 133 similar to as described elsewhere herein. The variable stiffness layer (e.g., braid layer) can be, for example, 0.001″ to 0.040″ thick. For example, a variable stiffness layer can be 0.001″, 0.003″, 0.005″, 0.010″, 0.015″, 0.020″, 0.025″ or 0.030″ thick. In some examples, as shown in FIG. 3D, the braid can have tensile or hoop fibers 137. Hoop fibers 137 can be spiraled and/or woven into a braid layer. Further, the hoop fibers 137 can be positioned at 2-50, e.g., 20-40 hoops per inch. The hoop fibers 137 can advantageously deliver high compression stiffness (to resist buckling or bowing out) in the radial direction but can remain compliant in the direction of the longitudinal axis 135 of the rigidizing device 100. That is, if compression is applied to the rigidizing device 100, the braid layer 109 will try to expand in diameter as it compresses. The hoop fibers 137 can resist this diametrical expansion and thus resist compression. Accordingly, the hoop fiber 137 can provide a system that is flexible in bending but still resists both tension and compression.

In some examples, the rigidizing devices described herein can have more than one variable stiffness layer. For example, the rigidizing devices can include two, three, or four variable stiffness layers. Referring to FIG. 3E, the layer 105 can be a second braid layer 105. The second braid layer 105 can have any of the characteristics described with respect to the first braid layer 109. In some examples, the braid of second braid layer 105 can be identical to the braid of first braid layer 109. In other examples, the braid of second braid layer 105 can be different than the braid of the first braid layer 109. For example, the braid of the second braid layer 105 can include fewer strands and have a larger braid angle α than the braid of the first braid layer 109. Having fewer strands can help increase the flexibility of the rigidizing device 100 (relative to having a second strand with equivalent or greater number of strands), and a larger braid angle α can help constrict the diameter of the of the first braid layer 109 (for instance, if the first braid layer is compressed) while increasing/maintaining the flexibility of the rigidizing device 100. As another example, the braid of the second braid layer 105 can include more strands and have a larger braid angle α than the braid of the first braid layer 109. Having more strands can result in a relatively tough and smooth layer while having a larger braid angle α can help constrict the diameter of the first braid layer 109.

The layer 103 can be another radial gap layer similar to layer 111. The gap layer 103 can have a thickness of 0.0002-0.04″, such as approximately 0.03″. A thickness within this range can ensure that the strands 133 of the braid layer(s) can easily slip and/or bulge relative to one another to ensure flexibility during bending of the rigidizing device 100.

The outermost layer 101 can be configured to move radially inward when a vacuum is applied to pull down against the braid layers 105, 109 and conform onto the surface(s) thereof. The outermost layer 101 can be soft and atraumatic and can be sealed at both ends to create a vacuum-tight chamber with layer 115. The outermost layer 101 can be elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

In some examples, the outermost layer 101 can, for example, have tensile or hoop fibers 137 extending therethrough. The hoop fibers 137 can be made, for example, of aramids (e.g., Technora, nylon, Kevlar), Vectran, Dyneema, carbon fiber, fiber glass or plastic. Further, the hoop fibers 137 can be positioned at 2-50, e.g., 20-40 hoops per inch. In some examples, the hoop fibers 137 can be laminated within an elastomeric sheath. The hoop fibers can advantageously deliver higher stiffness in one direction compared to another (e.g., can be very stiff in the hoop direction, but very compliant in the direction of the longitudinal axis of the rigidizing device). Additionally, the hoop fibers can advantageously provide low hoop stiffness until the fibers are placed under a tensile load, at which point the hoop fibers can suddenly exhibit high hoop stiffness.

In some examples, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, painting, or spraying the coating thereon.

The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

In some examples, the outermost layer 101 can be loose over the radially inward layers. For instance, the inside diameter of layer 101 (assuming it constitutes a tube) may have a diametrical gap of 0″-0.200″ with the next layer radially inwards (e.g., with a braid layer). This may give the vacuum rigidized system more flexibility when not under vacuum while still preserving a high rigidization multiple. In other examples, the outermost layer 101 may be stretched some over the next layer radially inwards (e.g., the braid layer). For instance, the zero-strain diameter of a tube constituting layer 101 may be from 0-0.200″ smaller in diameter than the next layer radially inwards and then stretched thereover. When not under vacuum, this system may have less flexibility than one wherein the outer layer 101 is looser. However, it may also have a smoother outer appearance and be less likely to tear during use.

In some examples, the outermost layer 101 can be loose over the radially inward layers. A small positive pressure may be applied underneath the layer 101 in order to gently expand layer 101 and allow the rigidizing device to bend more freely in the flexible configuration. In this example, the outermost layer 101 can be elastomeric and can maintain a compressive force over the braid, thereby imparting stiffness. Once positive pressure is supplied (enough to nominally expand the sheath off of the braid, for example, 2 psi), the outermost layer 101 is no longer is a contributor to stiffness, which can enhance baseline flexibility. Once rigidization is desired, positive pressure can be replaced by negative pressure (vacuum) to deliver stiffness.

A vacuum can be carried within rigidizing device 100 from minimal to full atmospheric vacuum (e.g., approximately 14.7 psi). In some examples, there can be a bleed valve, regulator, or pump control such that vacuum is bled down to any intermediate level to provide a variable stiffness capability. The vacuum pressure can advantageously be used to rigidize the rigidizing device structure by compressing the layer(s) of braided sleeve against neighboring layers. Braid is naturally flexible in bending (i.e. when bent normal to its longitudinal axis), and the lattice structure formed by the interlaced strands distort as the sleeve is bent in order for the braid to conform to the bent shape while resting on the inner layers. This results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some examples, the hoop fibers through or over the braid can carry tensile loads that help to prevent local buckling of the braid at high applied bending load.

The stiffness of the rigidizing device 100 can increase from 2-fold to over 30-fold, for instance 10-fold, 15-fold, or 20-fold, when transitioned from the flexible configuration to the rigid configuration. In one specific example, the stiffness of a rigidizing device similar to rigidizing device 100 was tested. The wall thickness of the test rigidizing device was 1.0 mm, the outer diameter was 17 mm, and a force was applied at the end of a 9.5 cm long cantilevered portion of the rigidizing device until the rigidizing device deflected 10 degrees. The forced required to do so when in flexible mode was only 30 grams while the forced required to do so in rigid (vacuum) mode was 350 grams.

In some examples of a vacuum rigidizing device 100, there can be only one braid layer. In other examples of a vacuum rigidizing device 100, there can be two, three, or more braid layers. In some examples, one or more of the radial gap layers or slip layers of rigidizing device 100 can be removed. In some examples, some or all of the slip layers of the rigidizing device 100 can be removed.

The braid layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. Other variable stiffness elements can be used in addition to or in place of the braid layer. In some examples, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, longitudinal members or substantially longitudinal members.

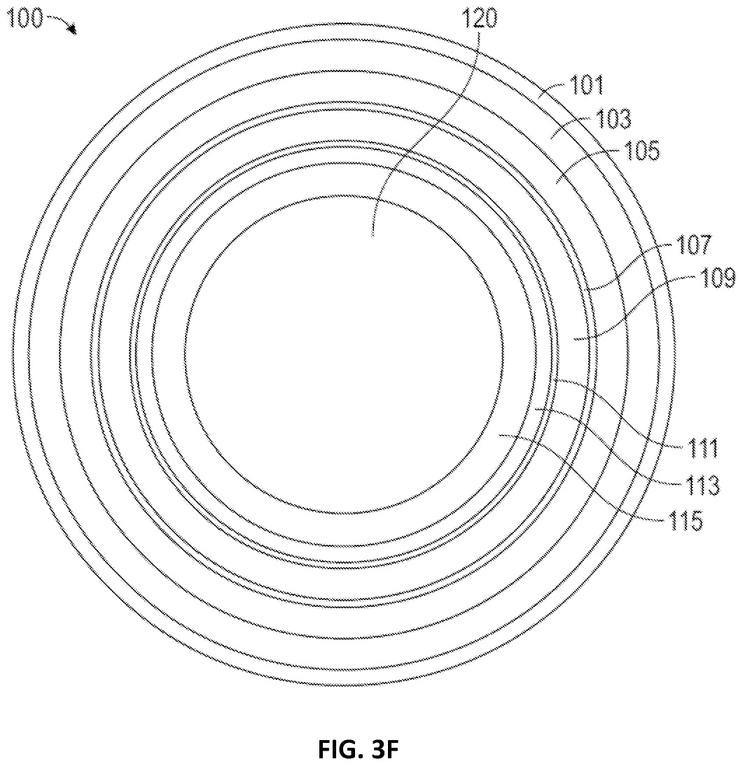

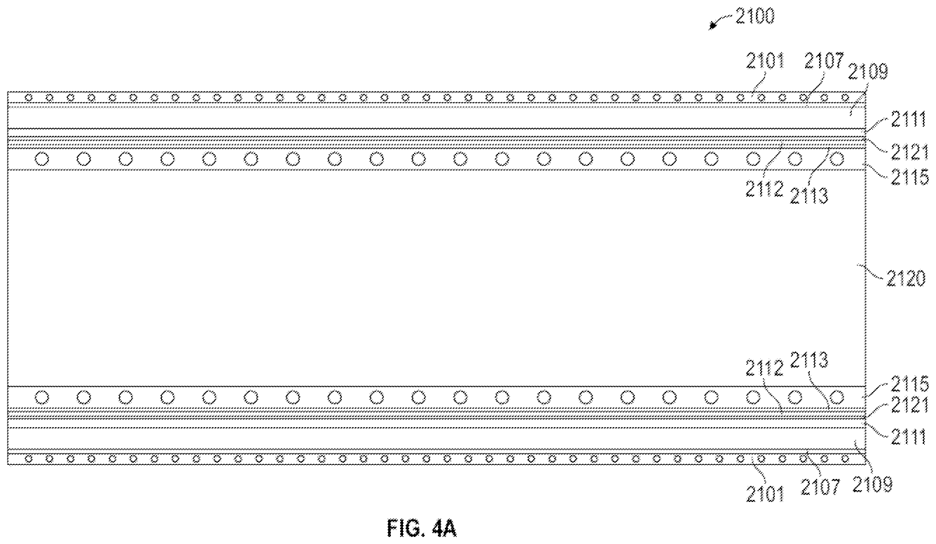

The rigidizable apparatuses described herein may also be rigidized by the application of positive pressure, rather than vacuum. For example, referring to FIGS. 4A-4B, the rigidizing apparatus (e.g., device or system) 2100 can be similar to rigidizing apparatus 100 described above, except that it can be configured to hold pressure (e.g., of greater than 1 atm) therein for rigidization rather than vacuum. A pressure-activated rigidizing device 2100 can also include a plurality of layers positioned around a lumen 2120 (e.g., for placement of an instrument or endoscope therethrough).

For example, FIGS. 4A-4B illustrate longitudinal and radial sections through an example of a pressure-activated rigidizable member of a rigidizing apparatus. The rigidizing device 2100 shown in FIGS. 4A and 4B can include an innermost layer 2115 (similar to innermost layer 115), an optional slip layer 2113 (similar to slip layer 113), a pressure gap 2112, a bladder layer 2121, a gap layer 2111 (similar to gap layer 111), a variable stiffness layer 2109 (similar to variable stiffness layer 109), a gap layer 2107 (similar to layer 107), and an outermost (e.g., containment) layer 2101.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some examples, the pressure gap 2112 of the rigidizing device 2100 can be connected to a pre-filled pressure source, such as a pre-filled syringe or a pre-filled insufflator, thereby reducing the physician's required set-up time.

The bladder layer 2121 can be made, for example, of a low durometer elastomer (e.g., of shore 20 A to 70 A) or a thin plastic sheet. The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some examples, the bladder layer 2121 can be 0.0002-0.020″ thick, such as approximately 0.005″ thick. The bladder layer 2121 can be soft, high-friction, stretchy, and/or able to wrinkle easily. In some examples, the bladder layer 2121 is a polyolefin or a PET. The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the braid layer 2109 against the outermost containment layer 2101 such that the relative motion of the braid strands is reduced.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other examples described herein. In some examples, the outermost containment layer 2101 can include a helical spring (e.g., made of circular or flat wire), and/or a tubular braid (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 include reinforcement elements therein, the braid layer 2109 can be reasonably constrained from both shrinking diameter (under tensile loads) and growing in diameter (under compression loads).

By using pressure rather than vacuum to transition from the flexible state to the rigid state, the rigidity of the rigidizing device 2100 can be increased. For example, in some examples, the pressure supplied to the pressure gap 2112 can be between 1 and 40 atmospheres, such as between 2 and 40 atmospheres, such as between 4 and 20 atmospheres, such as between 5 and 10 atmospheres. In some examples, the pressure supplied is approximate 2 atm, approximately 4 atmospheres, approximately 5 atmospheres, approximately 10 atmospheres, approximately 20 atmospheres. In some examples, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

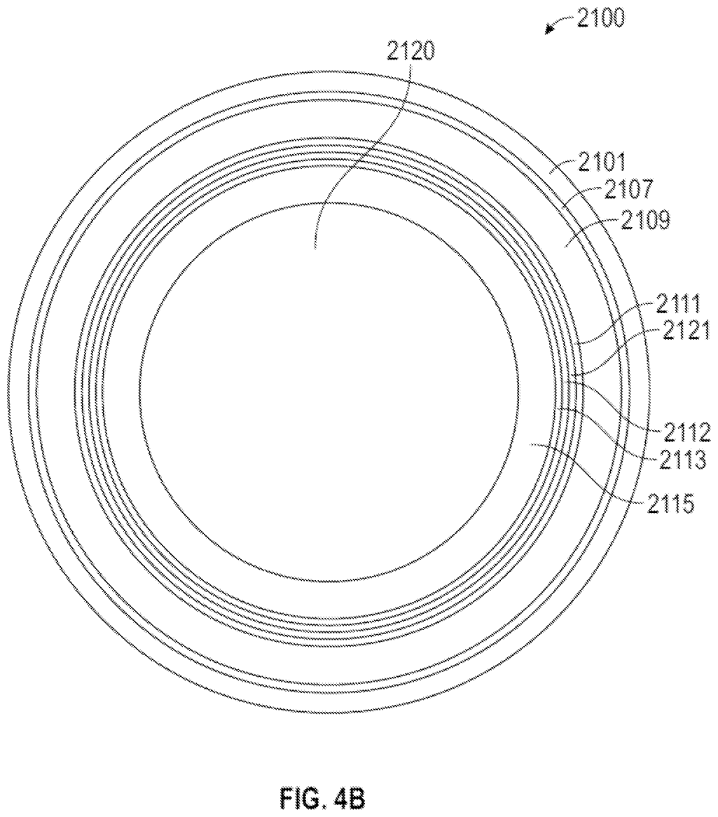

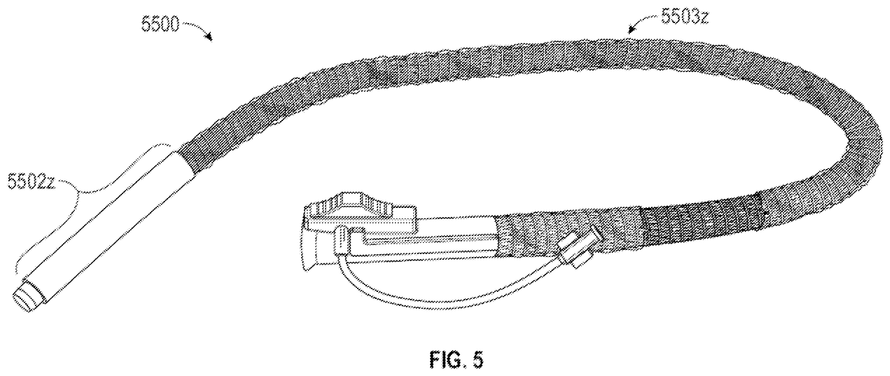

Any of the rigidizing devices described herein can have a distal end section or sections with a different design that the main elongate body of the rigidizing device. As shown in FIG. 5, for example, rigidizing device 5500 can have a main elongate body 5503z and a distal end section 5502z. Only the distal end section 5502z, only the main elongate body 5503z, or both the distal end section 5502z and the main elongate body 5503z can be rigidizing as described herein (e.g., by vacuum and/or pressure). In some examples, one section 5502z, 5503z is activated by pressure and the other section 5502z, 5503z is activated by vacuum. In other examples, both sections 5502z, 5503z are activated by pressure or vacuum, respectively.

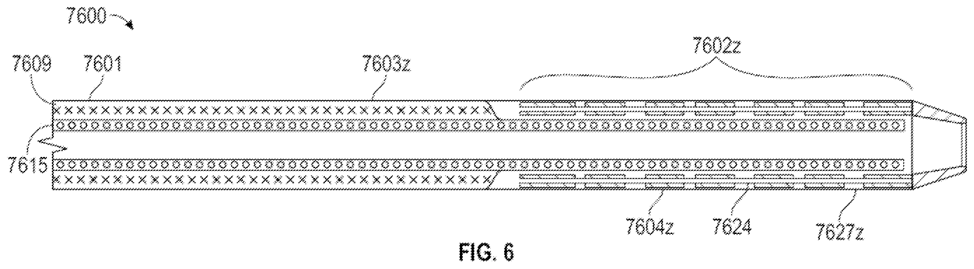

Referring to FIG. 6, in other examples, the distal end section 7602z can include a plurality of linkages 7604z that are actively controlled, such as via cables 7624, for steering of the rigidizing device 7600. The device 7600 is similar to device 5800 except that it includes cables 7624 configured to control movement of the device. While the passage of the cables 7624 through the rigidizing elongate body 7603z (i.e., with outer wall 7601, braid layer 7609, and inner layer 7615) is not shown in FIG. 6, the cables 7624 can extend therethrough in any manner as described elsewhere herein. In some examples, one or more layers of the rigidizing elongate body 7603z can continue into the distal end section 7602z. For example, and as shown in FIG. 26, the inner layer 7615 can continue into the distal end section 7602z, e.g., can be located radially inwards of the linkages 7604z. Similarly, any of the additional layers from the rigidizing proximal section (e.g., the braid layer 7609 or the outer layer 7601 may be continued into the distal section 7602z and/or be positioned radially inwards of the linkages 7604z). In other examples, none of the layers of the rigidizing elongate body 7603z continue into the distal section 7602z. The linkages 7604z (and any linkages described herein) can include a covering 7627z thereover. The covering 7627z can advantageously make the distal section 7602z atraumatic and/or smooth. The covering 7627z can be a film, such as expanded PTFE. Expanded PTFE can advantageously provide a smooth, low friction surface with low resistance to bending but high resistance to buckling.

In some examples, the rigidizing devices described herein can be used in conjunction with one or more other rigidizing devices described herein. For example, an endoscope can include the rigidizing mechanisms described herein, and a rigidizing device can include the rigidizing mechanisms described herein. Used together, they can create a nested system that can advance, one after the other, allowing one of the elements to always remain stiffened, such that looping is reduced or eliminated (i.e., they can create a sequentially advancing nested system).

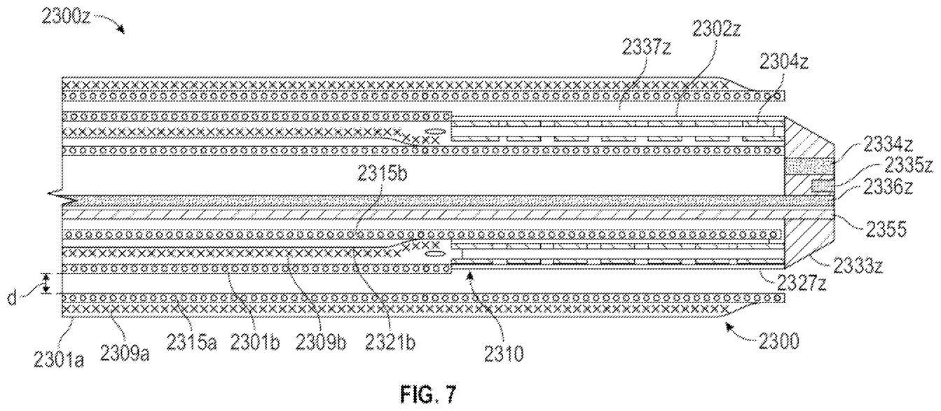

An exemplary nested system 2300z is shown in FIG. 7. The system 2300z can include an outer rigidizing device 2300 and an inner rigidizing device 2310 (here, configured as a rigidizing scope) that are axially movable with respect to one another either concentrically or non-concentrically. The outer rigidizing device 2300 and the inner rigidizing device 2310 can include any of the rigidizing features as described herein. For example, the outer rigidizing device 2300 can include an outermost layer 2301a, a variable stiffness layer 2309a, and an inner layer 2315a including a reinforcing coil wound therethrough. The outer rigidizing device 2300 can be, for example, configured to receive vacuum between the outermost layer 2301a and the inner layer 2315a to provide rigidization. Similarly, the inner scope 2310 can include an outer layer 2301b (e.g., with a coil wound therethrough), a variable stiffness layer 2309b, a bladder layer 2321b, and an inner layer 2315b (e.g., with a coil wound therethrough). The inner scope 2310 can be, for example, configured to receive pressure between the bladder 2321b and the inner layer 2315b to provide rigidization. Further, an air/water channel 2336z and a working channel 2355 can extend through the inner rigidizing device 2310. Additionally, the inner rigidizing scope 2310 can include a distal section 2302z with a camera 2334z, lights 2335z, and steerable linkages 2304z. A cover 2327z can extend over the distal section 2302z. In another example, the camera and/or lighting can be delivered in a separate assembly (e.g., the camera and lighting can be bundled together in a catheter and delivered down the working channel 2355 and/or an additional working channel to the distalmost end 2333z).

An interface 2337z can be positioned between the inner rigidizing device 2310 and the outer rigidizing device 2300. The interface 2337z can be a gap, for example, having a dimension d (see FIG. 5) of 0.001″-0.050″, such as 0.0020″, 0.005″, or 0.020″ thick. In some examples, the interface 2337z can be low friction and include, for example, powder, coatings, or laminations to reduce the friction. In some examples, there can be seals between the inner rigidizing device 2310 and outer rigidizing device 2300, and the intervening space can be pressurized, for example, with fluid or water, to create a hydrostatic bearing. In other examples, there can be seals between the inner rigidizing device 2310 and outer rigidizing device 2300, and the intervening space can be filled with small spheres to reduce friction.

The inner rigidizing device 2310 and outer rigidizing device 2300 can move relative to one another and alternately rigidize so as to transfer a bend or shape down the length of the nested system 2300z. For example, the inner device 2310 can be inserted into a lumen and bent or steered into the desired shape. Pressure can be applied to the inner rigidizing device 2310 to cause the variable stiffness layer(s) to engage and lock the inner rigidizing device 2310 in the configuration. The rigidizing device (for instance, in a flexible state) 2300 can then be advanced over the rigid inner device 2310. When the outer rigidizing device 2300 reaches the tip of the inner device 2310, vacuum can be applied to the rigidizing device 2300 to cause the layers to engage and lock to fix the shape of the rigidizing device. The inner device 2310 can be transitioned to a flexible state, advanced, and the process repeated. Although the system 2300z is described as including a rigidizing device and an inner device configured as a scope, it should be understood that other configurations are possible. For example, the system might include two overtubes, two catheters, or a combination of overtube, catheter, and scope. Both the inner and outer members are rigidizing members in these examples.

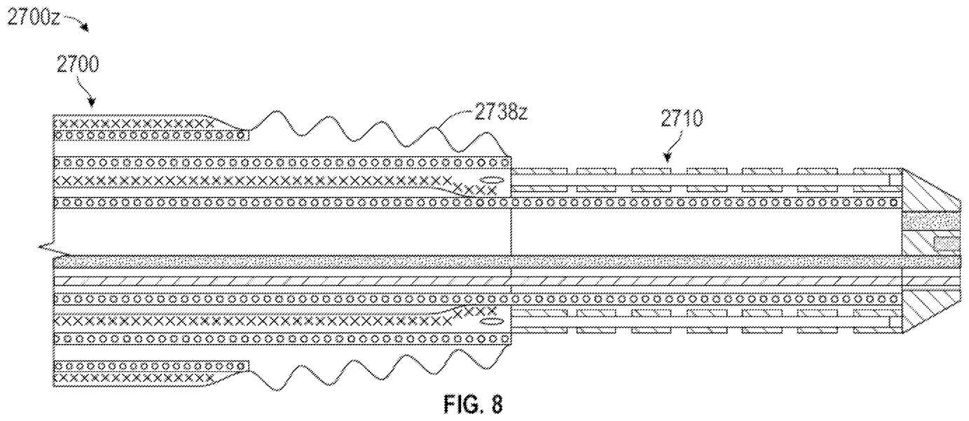

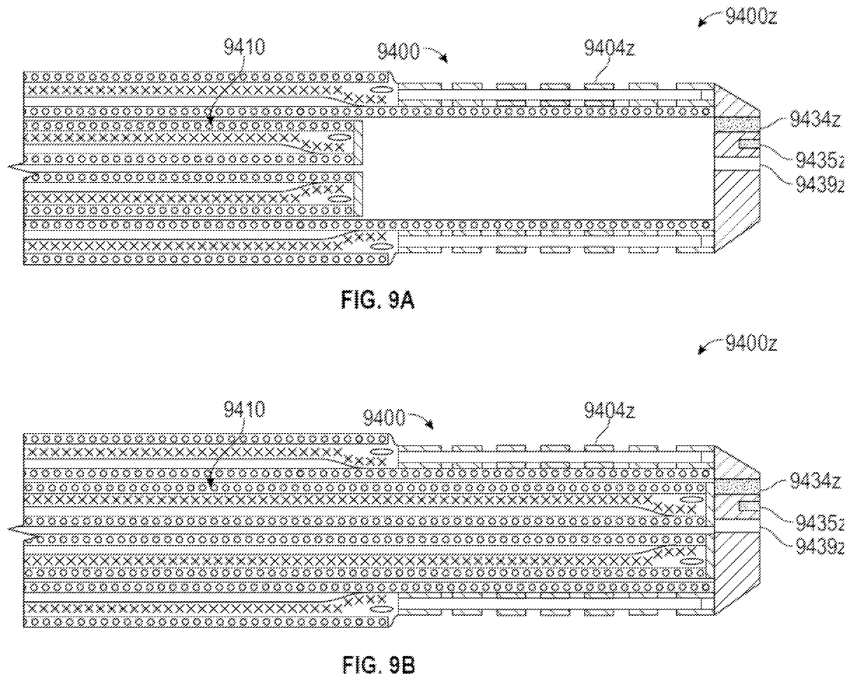

FIG. 8 shows another exemplary nested system 2700z. System 2700z is similar to system 2300z except that it includes a cover 2738z attached to both the inner and outer rigidizing device 2710, 2700. The cover 2738z may be, for example, low-durometer and thin-walled to allow elasticity and stretching. The cover 2738z may be a rubber, such as urethane, latex, or silicone. The cover 2738z may protect the interface/radial gap between the inner and outer devices 2710, 2700. The cover 2738z may prevent contamination from entering the space between the inner and outer tubes. The cover 2738z may further prevent tissue and other substances from becoming trapped in the space between the inner and outer tubes. The cover 2738z may stretch to allow the inner device 2710 and outer device 2700 to travel independently of one another within the elastic limits of the material. The cover 2738z may be bonded or attached to the rigidizing devices 2710, 2700 in such a way that the cover 2738z is always at a minimum slightly stretched. This example may be wiped down externally for cleaning. In some examples, the cover 2738z can be configured as a “rolling” seal, such as disclosed in U.S. Pat. No. 6,447,491, the entire disclosure of which is incorporated by reference herein.

FIGS. 9A-9B show another exemplary nested system 9400z. In this system 9400z, the outer rigidizing device 9400 includes steering and imaging (e.g., similar to a scope) while the inner device includes only rigidization (though it could include additional steering elements as described elsewhere herein). Thus, outer device 9400 includes linkages or other steering means disclosed herein 9404z, camera 9434z, and lighting 9435z. The outer device 9400 can further include a central passageway 9439z for access to the inner device 9410 (e.g., lumens such as working channels therein). In some examples, bellows or a loop of tubing can connect the passageway 9439z to lumens of the inner device 9410. Similar to the other nested systems, at least one of the devices 9410, 9400 can be rigidized at a time while the other can conform to the rigidization and/or move through the anatomy. Here, the outer device 9400 can lead the inner device 9410 (the inner device 9410 is shown retracted relative to the outer device 9400 in FIG. 9A and extended substantially even with the outer device 9400 in FIG. 7B). Advantageously, system 9400z can provide a smooth exterior surface to avoid pinching the anatomy and/or entrance of fluid between the inner and outer devices 9410, 9400. Having the steering on the outer device 9400 can also provide additional leverage for steering the tip. Also, the outer device can facilitate better imaging capabilities due to the larger diameter of the outer device 9400 and its ability to accommodate a larger camera.

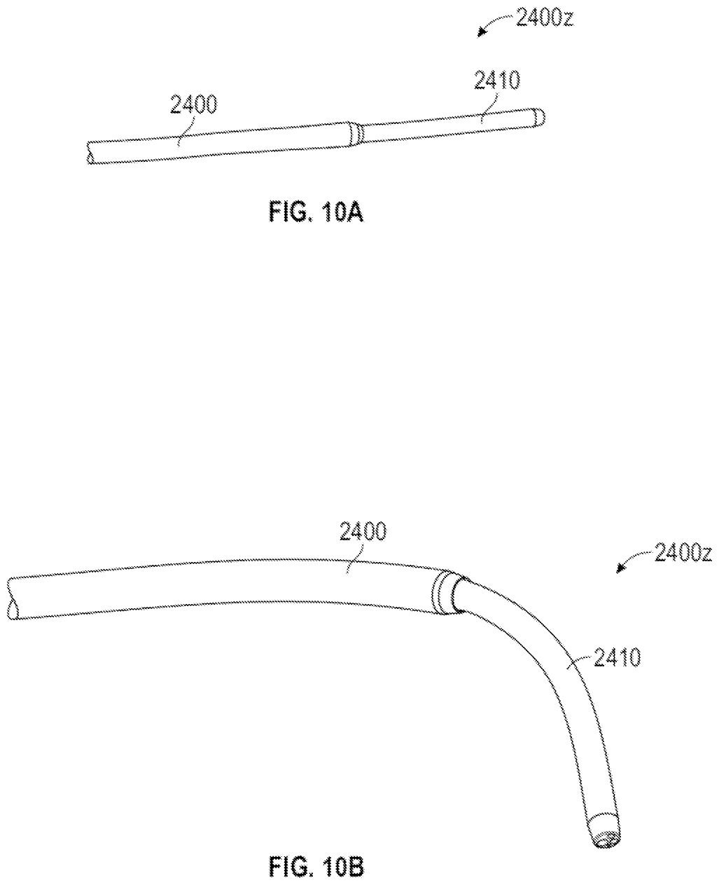

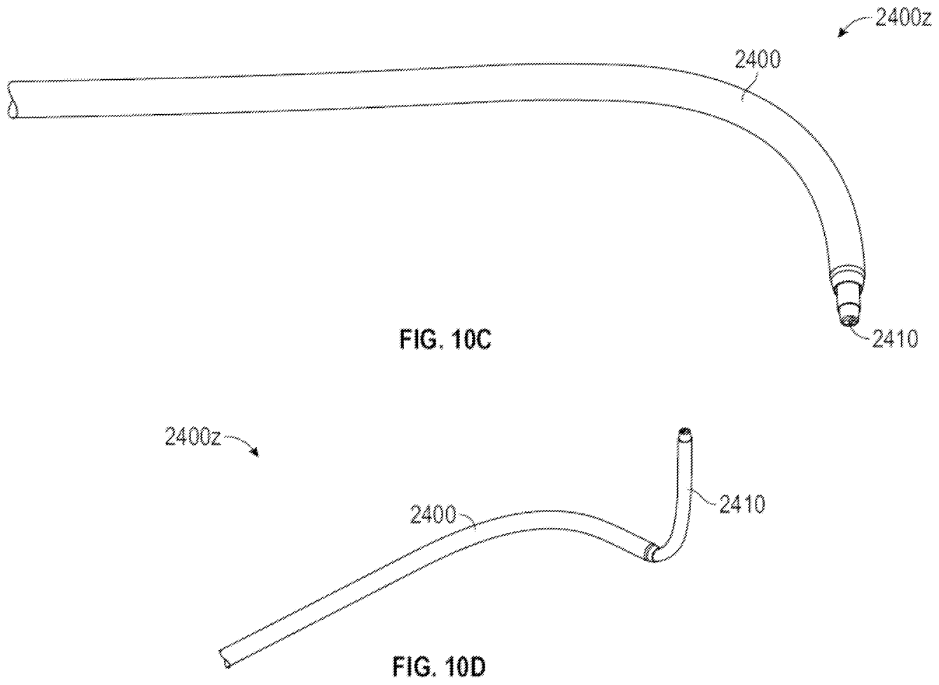

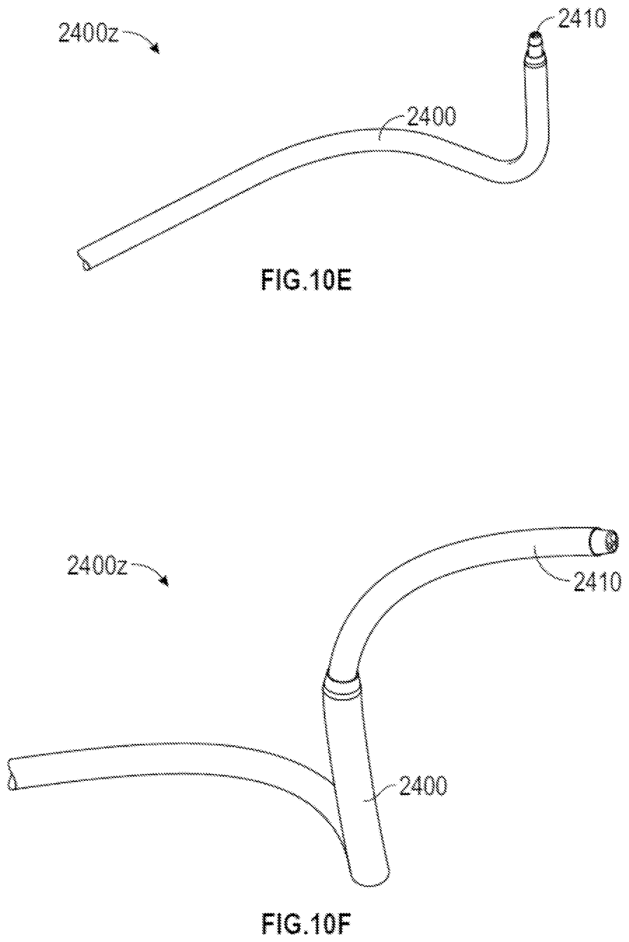

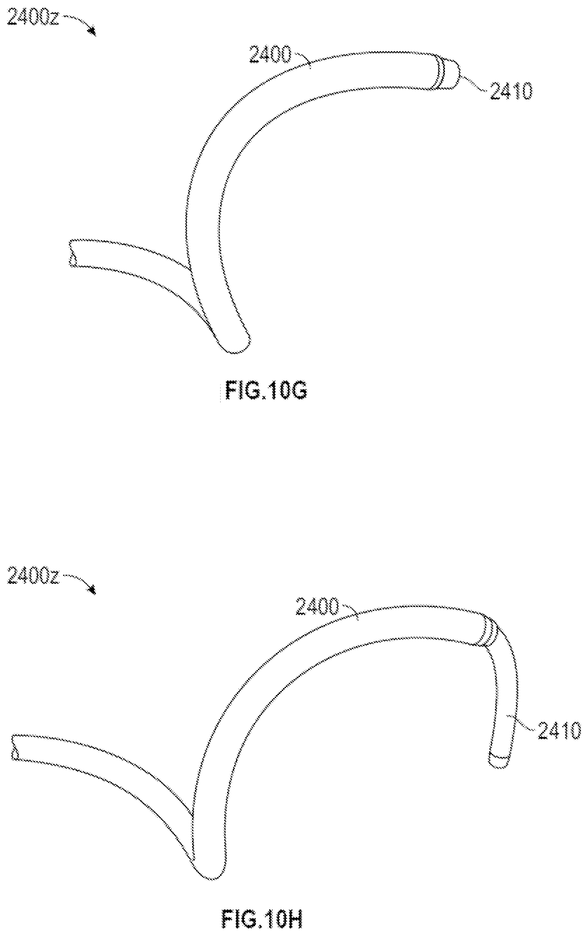

FIGS. 10A-10H show the exemplary use of a nested system 2400z as described herein. At FIG. 10A, a steerable inner rigidizing device 2410 is positioned within the outer rigidizing device 2400 such that the distal end of the inner rigidizing device 2410 extends outside of the outer rigidizing device 2400. At FIG. 10B, the distal end of the inner rigidizing device 2410 is bent in the desired direction/orientation (e.g., via steering cables, such as cables 7624) and then rigidized (e.g., using vacuum or pressure as described herein). At FIG. 10C, the outer rigidizing device 2400 (in the flexible configuration) is advanced over the rigidized inner rigidizing device 2410 (including over the bending distal section). Once the distal end of the outer rigidizing device 2400 is sufficiently advanced over the distal end of the inner rigidizing device 2410, then the outer rigidizing device 2400 can be rigidized (e.g., using vacuum or pressure as described herein). At FIG. 10D, the inner rigidizing device 2410 can then be transitioned to the flexible state (e.g., by removing the vacuum or pressure as described herein and by allowing the steering cables to go slack such that tip can move easily) and can be advanced and directed/oriented/steered as desired. Alternately, in FIG. 10D, the inner rigidizing device 2410 can be actively steered (either manually or via computational control) as it emerges such that is minimizes the load on the rigidized outer tube. Minimizing the load on the outer rigidizing device 2400 makes it easier for this tube to hold the rigidized shape. Once the inner rigidizing device 2410 is rigidized, the outer rigidizing device 2400 can be transitioned to the flexible state and advanced thereover (as shown in FIG. 10E). The process can then be repeated as shown in FIGS. 10F-H. The repeated process can result in “shape copying,” whereby the inner and outer rigidizing devices 2410, 2400 in the flexible configuration continuously conform to (or copy) the shape of whichever device 2410, 2400 is in the rigid configuration.

In some examples, at the completion of the sequence shown in FIGS. 10A-H, a third rigidizing device can be slid over the first two rigidizing devices (2400, 2410) and rigidized. Rigidizing devices 2400 and 2410 can then be withdrawn. Finally, a fourth rigidizing device can be inserted through the inner lumen of the third tube. This fourth rigidizing device may have a larger diameter and more features than rigidizing device 2410. For instance, it may have a larger working channel, more working channels, a better camera, or combinations thereof. This technique can allow two smaller tubes, which tend to be more flexible and maneuverable, to reach deep into the body while still ultimately deliver a larger tube for therapeutic purposes. Alternately, in the example above, the fourth rigidizing device can be a regular endoscope as is known in the art.

In some examples, at the completion of the sequence shown in FIGS. 10A-H, outer rigidizing device 2400 may be rigidized and then the inner rigidizing device 2410 may be removed. For example, the rigidizing device 2410 may be a “navigation” device comprising a camera, lighting and a distal steering section. The “navigation” device 2410 may be well sealed such that it is easy to clean between procedures. A second inner device may then be placed inside the rigidized outer device 2400 and advanced past the distal end of the outer device 2400. The second inner device may be a “therapeutic” tube comprising such elements as a camera, lights, water, suction and various tools. The “therapeutic” device may not have a steering section or the ability to rigidize, thereby giving additional room in the body of the therapeutic tube for the inclusion of other features, for example, tools for performing therapies. Once in place, the tools on the “therapeutic” tube may be used to perform a therapy in the body, such as, for example, a mucosal resection or dissection in the human GI tract.

In another example, after or during the completion of the sequence shown in FIGS. 10A-H, a third device may be inserted inside inner tube 2410. The third device may be rigidizing and/or an endoscope.

In some examples, after completion of the sequence shown in FIGS. 10A-10H and the completion of any therapies conducted with the system 2400z in place, the entire system 2400z can be removed from the anatomy. In one exemplary method of withdrawing, the system 2400z can be transitioned to the flexible configuration (i.e., both the inner and outer devices 2410, 2400 can be transitioned to the flexible configuration), and the flexible system 2400z can be pulled proximally. In this method, the tension between the patient's body (e.g., the anus) and a robotic arm (e.g., arm 1023y described below) can prevent the system 2400z from falling out of the body as it is removed (e.g., as more of the flexible system 2400z is positioned outside of the body than inside of the body).

As another exemplary method of withdrawing, shape copying can be performed similar to as described with respect to FIGS. 10A-10H, but in reverse. In this example, for example, the inner rigidizing device 2410 can be rigidized and the outer rigidizing device 2400 can be withdrawn proximally (while in the flexible configuration) over the inner rigidizing device 2410. The outer rigidizing device 2400 can then be rigidized and the inner rigidizing device 2410 can be relaxed and moved proximally within the outer rigidizing device 2400 (e.g., until the distal end of the inner rigidizing device 2410 is flush with the distal end of the outer rigidizing device 2400). In this example, when the inner rigidizing device 2410 is withdrawn into the outer rigidizing device 2400, tension on the steering cables can be held constant (e.g., at a low value, such as ¼ lb or less) to ensure that the steerable distal end section will move into the shape of the outer rigidizing device 2400 without disturbing the fixed shape of the outer rigidizing device 2400. Alternatively or additionally, if the outer rigidizing device 2400 is rigidized in a straight shape, then the inner rigidizing device 2410 can be pulled into the outer rigidizing device 2400 and tension on each of the steering cables can be made equal (i.e., the same value, thus conforming the child shape to shape of the inside of the mother).

As another exemplary method of withdrawing, the steerable distal tip of the inner rigidizing device 2410 can be actively steered proximally into the known, assumed, or measured shape of the outer rigidizing device 2400 either as or after the distal tip is retracted into the outer rigidizing device 2410. That is, the distal tip of the inner rigidizing device 2410 can be steered to match the shape of the section of the outer rigidizing device 2400 that is immediately proximal to the distal tip of the inner rigidizing device 2410. In one specific example, the inner rigidizing device 2410 may project from the outer rigidizing device 2400 by 4 inches, and the last 4 inches of the outer rigidizing device 2400 may form a 90 degree curve around a 2.5 inch radius of curvature. In this example, the inner rigidizing device 2410 can be steered into a 90 degree curve around a 2.5 inch radius of curvature and then withdrawn (in that shape) into the outer rigidizing device 2400. This may advantageously ensure that the inner rigidizing device 2410 pulls easily into the outer rigidizing device 2400 (i.e., because their shapes are matched).

Both the rigidity and the movement (e.g., rotation in the clockwise direction, rotation in the counter clockwise direction, advancing distally, retracing proximally) may be separately controlled for the inner and outer rigidizing members. In some examples either or both the inner and outer members may also be steerable. For example, either the inner or the outer or both the inner and outer rigidizing members may be steerable at their distal ends. In some cases, steering may be controlled by one or more wires (e.g., tendons). As will be described in greater detail herein, the rigidity and movement, including steering of the distal end, may be controlled and/or coordinated by a controller to perform the methods described herein, including reductions. The controller may also receive input from one or more shape sensor in either or both the inner and outer rigidizing members. For example a fiber optic shape sensor may be part of either the inner or outer or both. Either or both the inner and outer rigidizing members may also include one or more force sensors detecting the force required to move the inner and/or outer member. Any of these apparatuses may include one or more torque sensors configured to detect torque, or resistance to rotation, on the inner rigidizing member in the clockwise and/or counterclockwise direction. Any of these apparatuses may include one or more torque sensors configured to detect torque, or resistance to rotation, on the outer rigidizing member in the clockwise and/or counterclockwise direction. In addition, the apparatus may include one or more sensors for detecting resistance to advancing and/or retracting the inner and/or outer rigidizing member. In general, the controllers described herein may coordinate movement, including reductions, based at least in part on input from these shape sensing and/or torque or other force sensors. The controllers described herein may also control and coordinate the rigidity of the inner and/or outer rigidizing members.

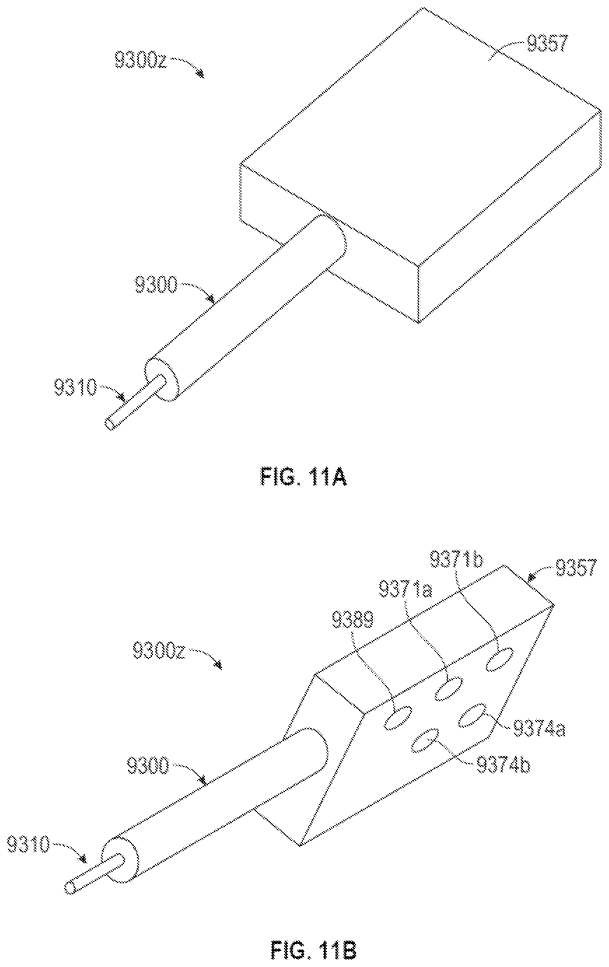

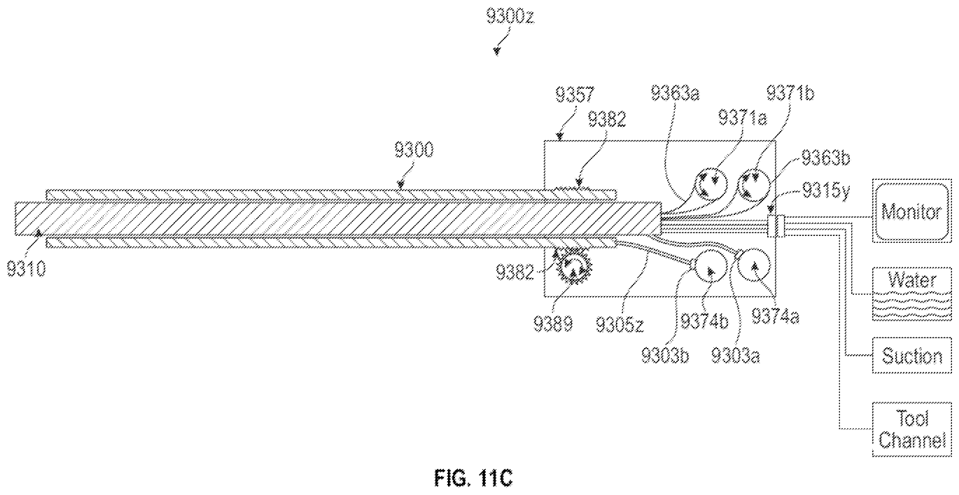

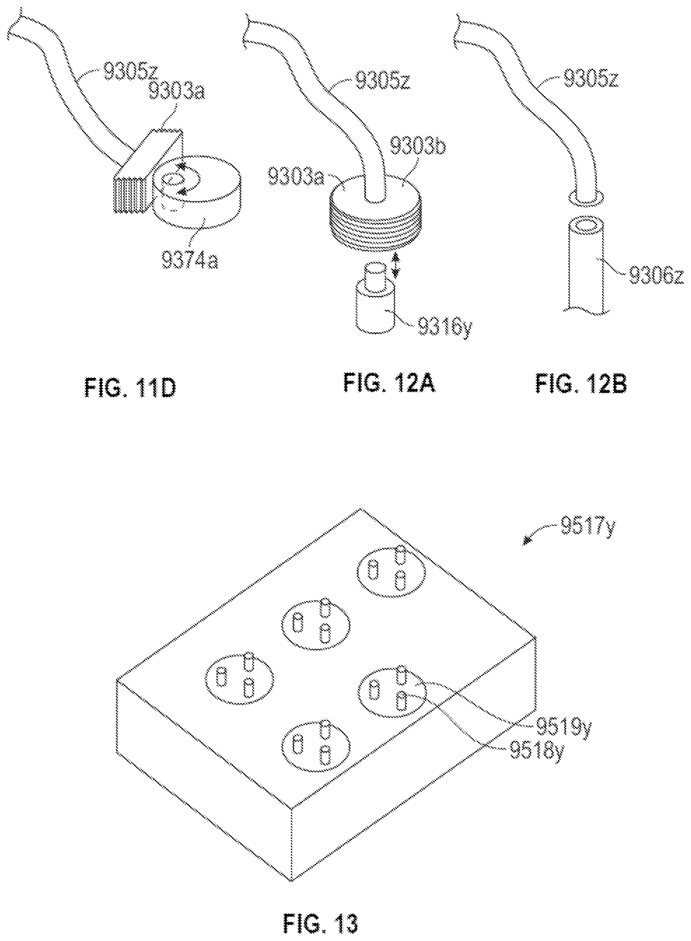

In some examples, the rigidizing devices (e.g., nested systems such as system 2400z) described herein can be robotically controlled. Any appropriate control subsystem may be used for controlling movement, including steering of the distal ends, advancing and/or retracting the rigidizing members, and/or rotating the inner and/or outer rigidizing members. FIGS. 11A-11D show an exemplary use of a nested system 9300z, like that shown in FIGS. 10A-10H, that can be robotically controlled or manipulated (e.g., for rigidization, steering, movement, rotation, etc.). As shown in FIGS. 11A-11D, the outer rigidizing device 9300 and the inner rigidizing device 9310 may be terminated together into a common structure, such as a cassette 9357. The outer rigidizing device 9300 can be movable with respect to the inner rigidizing device 9310 by rotation of a disk 9389 that is mounted to the cassette 9357. For example, the disk 9389 can be a pinion, and the outer rigidizing device 9300 may have a rack 9382 including a plurality of small teeth on the outside thereof. Rotating the disk 9389 against teeth 9382 may cause outer rigidizing device 9300 to advance forward or backward relative to the inner rigidizing device 9310. In some examples, the possible movement or translation of the rigidizing devices 9300, 9310 is limited by the size or design of the cassette 9357.

The cassette 9357 can further include additional disks 9371a, 9371b that may connect to cables 9363a,b respectively, to steer (e.g., bend or deflect) the tip of the inner rigidizing device 9310 (and/or outer rigidizing device 9300). Other steering mechanisms (e.g., pneumatics, hydraulics, shape memory alloys, EAP (electro-active polymers), or motors) are also possible. Again, in examples with different steering mechanisms, one or more disks in the cassette 9357 (e.g., disks 9371a, 9371b) may be used to actuate the steering. The cassette 9357 can further include bellows 9303a, 9303b that may connect to the pressure gap of the inner rigidizing device 9310 and the outer rigidizing device 9300, respectively. Compressing bellows 9303a, 9303b may drive fluid through pressure lines 9305z, causing the pressure in the pressure gap of the inner rigidizing devices 9310, 9300 to rise, causing the rigidizing devices 9310, 9300 to become rigid. Activation of the bellows 9303a, 9303b may be applied sequentially and/or simultaneously. As shown in FIGS. 9A-9D, the cassette 9357 can include eccentric cams 9374a,b to control bellows 9303a,b.

Alternatively, as shown in FIG. 12A, one or more linear actuators 9316y (e.g., on cassette 9357 or on drive unit 9517y) can be configured to actuate the bellows 9303a,b. As another alternative, the devices 9300, 9310 can be rigidized and de-rigidized through one or more sumps (as described herein) or pressure sources 9306z (e.g., via pressure line 9305z), as shown in FIG. 12B. Other mechanisms causing rigidization of the inner and outer rigidizing devices 9310, 9300 are also possible. For example, in some examples, cassette 9357 can include a syringe or other container comprising a fluid that can be delivered to the inner and outer rigidizing devices 9310, 9300 to add pressure for rigidization. In some examples, a syringe or other container can be used to draw fluid within the cassette 9357, creating a vacuum that can be applied to the inner and outer rigidizing devices 9310, 9300.

Referring back to FIGS. 11A-11D, the cassette 9357 can include a connector 9315y for connecting to additional lumens and/or wiring in the inner rigidizing device 9310. The connector 9315y may include a connection for the delivery of both suction and water to the tip of the inner rigidizing device 9310. The connector 9315y may include electrical connector to connect to a camera mounted to the tip of inner rigidizing device 9310 to an external monitor and/or video processing unit. The connector 9315y may include a mechanical connector that connects to a hollow tube (e.g., working channel) leading all the way to the tip of the inner rigidizing device 9310. By including the connector 9315y, the control of all components of the system 9300z can be performed with the cassette 9357.

Disks 9389, 9371a, 9371b and cams 9374a, 9374b (or the corresponding bellows) may be accessible from the bottom of the cassette 9357, as best shown in the side perspective view of FIG. 93B. Disks 9389, 9371a, 9371b and/or cams 9374a, 9374b may have features, such as splines, pins or teeth, to transmit torque. These features can allow the disks 9389, 9371a, 9371b and/or cams 9374a, 9374b to be manipulated (e.g., by a drive unit).

FIG. 13 shows an exemplary a drive unit 9517y that may be used to drive the disks 9389, 9371a, 9371b and/or cams 9374a, 9374b. For example, the drive unit 9517y can include drive paddles 9519y that may align with disks 9389, 9371a, 9371b and/or cams 9374a, 9374b of the cassette 9357. The drive paddles 9519y can be driven (i.e., rotated) by one or more motors of the drive unit 9517y so as to deliver torque to the disks 9389, 9371a, 9371b and/or cams 9374a, 9374b of the cassette 9357. The drive paddles 9519y can includes features 9518y (e.g., splines, pins, teeth, or the like) to transmit torque to the disks 9389, 9371a, 9371b and/or cams 9374a, 9374b of the cassette 9357. The drive unit 9517y may attach to the cassette 9357, for example, with clips, screws, or magnets.

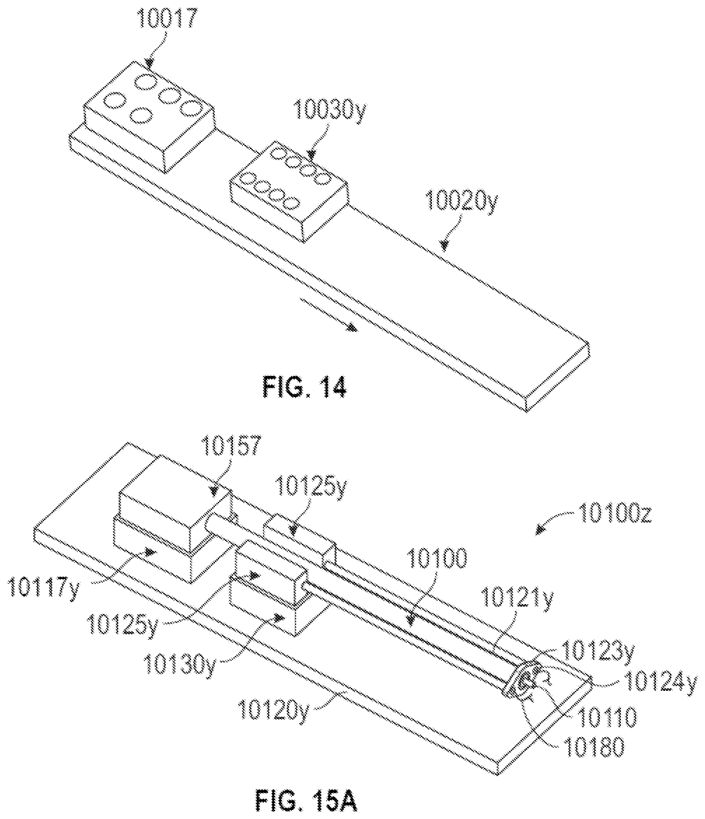

Referring to FIG. 14 and back to FIGS. 11A-11D, in some examples, the robotic system (e.g., system 9300z including the inner and outer rigidizing devices 9310, 9300 and cassette 9357) may be positioned on a linear slide 10020y. The linear slide 10020y can further include a drive unit 10017y (similar to drive unit 9517y) configured to control the inner and outer rigidizing devices 9310, 9300. The slide 10020y can allow the inner and outer rigidizing devices 9310, 9300 to be translated together (i.e., simultaneously). In some examples, in order to effect relative movement of the inner rigidizing device 9310 with respect to the outer rigidizing device 9300, the system 9300z can be translated in a first direction (forwards or backwards along the slide 10020y) while simultaneously using the disk 9389 and rack 9382 on the outer rigidizing device 9300 to move the outer rigidizing device 9300 in a second direction, opposite to the first direction. That is, to advance the inner rigidizing device 9310 relative to the outer rigidizing device 9300, the system 9300z including both rigidizing devices 9300, 9310 is advanced along the slide 10020y while simultaneously retracting the outer rigidizing device 9300 using the disk 9389 and rack 9382. Conversely, to retract the inner rigidizing device 9310 relative to the outer rigidizing device 9300, the system 9300z including both rigidizing devices 9300, 9310 can be retracted along the slide 10020y while simultaneously advancing the outer rigidizing device 9300.

In some examples, the drive unit 1017y, rack 9382, and/or disk 9389 can include a force gauge thereon. The force gauge can be configured to measure the amount of force required to insert or withdraw the inner and/or outer rigidizing devices 9310, 9300. The force gauge can be used, for example, to trigger one or more alarms if the amount of force has exceeded a threshold (e.g., a threshold amount of force that may cause injury to the anatomy if exceeded). In some examples, the one or more alarms can be escalating alarms. For example, a first alarm can indicate caution, and a second alarm can indicate probable danger of injury. In some examples, the threshold for the alarm(s) can vary depending on the state or mode of the system 9300z. For instance, when inch-worming forward by alternating inner member 9310 and outer member 9300 rigidity, the alarm(s) can have a first set of threshold values. In contrast, when withdrawing and moving backwards (e.g., wherein both the inner and outer members 9310, 9300 are flexible), the alarm(s) can have a second set of threshold values. The alarm(s) can be represented, for example, by a change in an image on a screen (e.g., a virtual light or color change), a change in the light (e.g., brightness or color) from one or more physical lights, a vibration or rumble in a controller, and/or the blocking of certain motions or functions. Although described as being on the drive unit 1017y, rack 9382, and/or disk 9389, it should be understood that the force gauge can be placed elsewhere within the system 9300z while still enabling measurement of insertion and/or withdrawal force.

Referring to FIG. 14, the linear slide 10020y can further include a second drive unit 10030y configured to control a tool or tools (e.g., tool 9980) used with the inner and outer rigidizing devices. In some examples, the first drive unit 10017y and the second drive unit 10030y can independently translate along linear slide 10020y. One, two or more tools 9980 may attach to drive unit 10030Y. The linear slide 10020y can advantageously ensure that the tool(s) used with the nested rigidizing system stay in place at the distal end of the outer rigidizing device despite any translation by the outer rigidizing device. For example, the tool drive unit 10030y can be configured to translate the tool forward when the outer rigidizing device advances relative to the slide 10020y. Similarly, the tool drive unit 10030y can be configured to retract the tool when the outer rigidizing device retracts relative to the slide 10020y. This may ensure, for example, that the tool stays locked into the fitting (e.g., fitting 9823y).

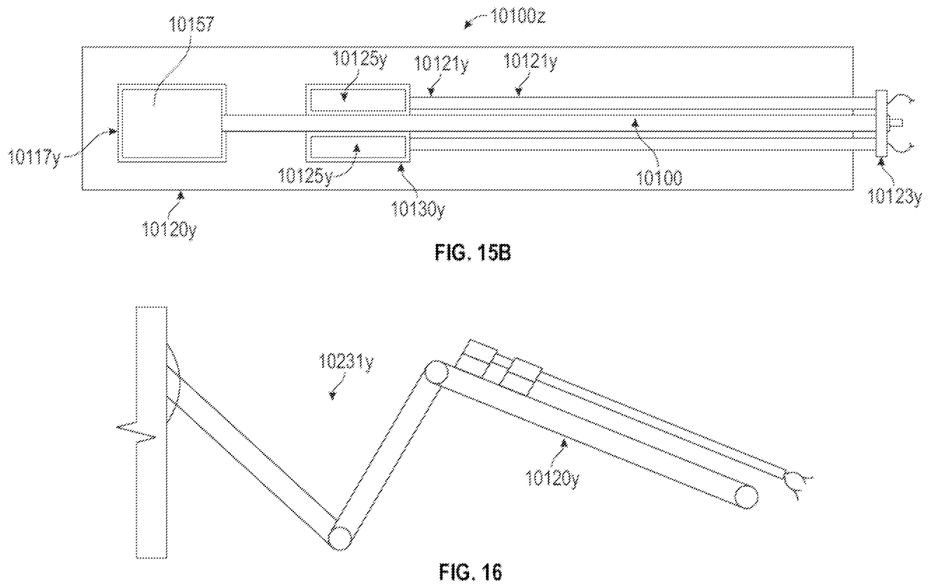

FIGS. 15A and 15B show top perspective and top views, respectively, of an exemplary robotic system 10100z positioned on a slide 10120y with cassette 10157 attached to a drive unit 10117y for control of the nested rigidizing devices 10100, 10110. Two cassettes 10125y for the control of two different tools 10180, are mounted to drive unit 10130y. The tools 10180 are inserted through guide 10121y and locked in fitting 10123y at ports 10124y.

FIG. 16 shows an exemplary pivoting arm 10231y that can be connected to the linear slide 10120y so as to orient the slide 10120y and thus the rest of the robotic system (including nested rigidizing devices 10100, 10110 and/or tools 10180) relative to the patient. As such, the linear slide 10120y may be positioned vertically, horizontally or at an angle in between.

The system 10100z may be used in the following exemplary manner. Cassette 10157 is attached to the inner and outer rigidizing devices 10110, 10100, and the inner and outer rigidizing devices 10110, 10100 are advanced into the patient's body (e.g., as detailed in FIGS. 65A-H). In some examples, the inner and outer rigidizing devices 10110, 10100 are advanced into the patient's colon or upper GI tract. Reciprocating motion of the inner rigidizing device 10110 and outer rigidizing device 10100 is provided by the motion of disk a disk within the cassette 10157 and the translation of the rigidizing devices 10110, 10100 along the slider 10120y. Rigidization is provided by compressing bellows in cassette 10157. Steering is provided by disks in cassette 10157. When a medical practitioner has reached the place in the body where the procedure is to be performed, a tool can be inserted through guide 10121y and locked to ports 10124y. The cassettes 10125y are then attached to drive unit 10130y for control of the tool.

The drive units described herein may be connected to a controller or control sub-system, which may include a computer (e.g., computer, tablet, laptop, etc.) for controlling and coordinating rigidity and movement of the apparatus, including for performing reductions as described herein. The controller in communication with the drive units may comprise software providing a user interface for a clinician to interact with to control the system and any tools being used. Automation, such as via computer controls of the cassettes and/or drive units described herein, can be used to make repetitive tasks easier to perform. For instance, a program can be developed that automatically moves the distal end of the rigidizing device in an arc while emitting water. A second arc can then be made to suction water and material from the GI tract. This may be useful, for example, in cleaning the GI tract. Software can be provided to perform, automatically or semi-automatically, any of the maneuvers including rigidization, described herein in sequence such that the operator needs only to provide input, with, for example, an input such as (but not limited to) a joystick, to direct the operation of the device, including the distal end of the device.

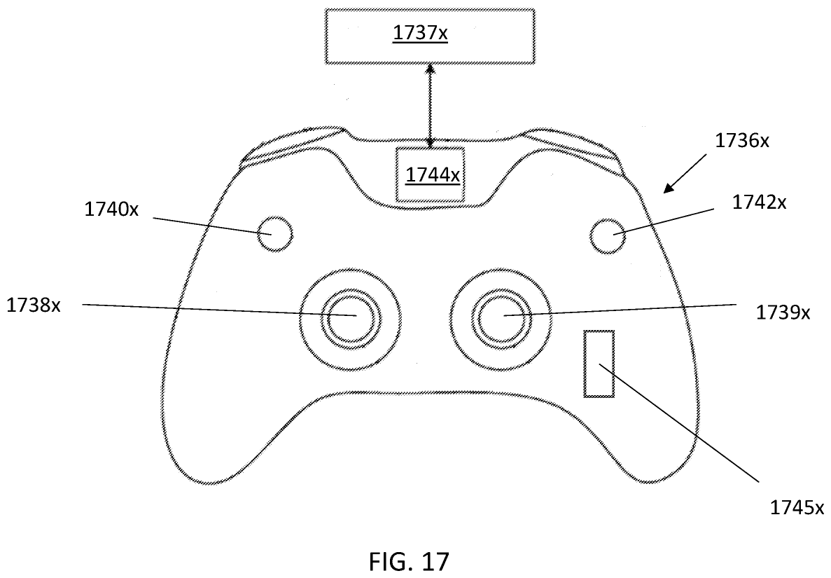

Any appropriate input may be used, including touchscreen, keyboard, joystick, pedal, etc. Referring to FIG. 17, in some examples, the control of system 2400z or 9300z (e.g., any of the sequences described herein, including as shown to FIGS. 10A-10H) can be performed using an input 1736x connected (e.g., wirelessly via onboard electronics 1744x) to a controller, which includes a computational device 1737x (i.e., a controller or computer). The input 1736x can be a hand-held input 1736x that include a steering actuator 1738x (e.g., toggle) configured to enable steering (e.g., up/down and left/right) of the inner rigidizing device 2410/9310. The input 1736x can further include a movement actuator 1739x (e.g., toggle) configured to enable forward/backward motion of the inner rigidizing device 2410/9310.

In some examples, the input 1736x can further include an actuator 1740x (e.g., button, knob, slider, etc.) configured to enable the outer rigidizing device 2400/9300 to automatically shape copy the inner rigidizing device 2410/9310. That is, upon activating the actuator 1740x (e.g., pushing the button), the inner rigidizing device 2410/9310 can transition from the flexible to the rigid configuration, then the outer rigidizing device 2400/9300 can transition from the rigid configuration to the flexible configuration, and the outer rigidizing device 2400/9300 can be advanced over the inner rigidizing device 2410/9310. In some examples, the advancement of the outer rigidizing device 2400/9300 can be terminated when the outer rigidizing device 2400/9300 reaches a preset position relative to the inner rigidizing device 9410/9310 (e.g., when the distal end of the outer rigidizing device 2400/9300 aligns with the distal end of the inner rigidizing device 2410/9310). In other examples, advancement of the outer rigidizing device 2400/9300 advancement can be terminated automatically by a sensor reading. For example, the sensor reading can be an output from a camera of the inner rigidizing device 2410/9310 (e.g., when the camera sees the outer rigidizing device 2400/9300 appear in the camera image). As another example, the sensor reading can be an amount of force that is required to advance the outer rigidizing device 2400/9300 (i.e., when the required force meets a pre-set threshold). When the outer rigidizing device 2400/9300 has completed its shape copying sequence, then the outer rigidizing device 2400/9300 can be automatically rigidized, and the inner rigidizing device 2410/9310 unrigidized to complete the automatic shape copying sequence. In some examples, the actuators 1738x, 1739x can be deactivated (i.e., to prevent movement of the inner rigidizing device 2410/9310) while the automatic shape copying sequence of the outer rigidizing device 2400/9300 (via activation of the actuator 1740x) is performed.

Although the steering and motion actuators 1738x, 1739x are shown in FIG. 17 as toggles and the automatic shape copying actuator 1740x as a button, it should be understood that any of the actuators could be replaced with alternative actuators (e.g., toggle, buttons, joysticks, slides, or track balls). Any of the actuators 1738x, 1739x, 1740x can include haptic feedback. For example, the activation of motion actuator 1739x can include a resistance to motion proportional to the amount of force required to move the inner rigidizing device 2410/9310 forward or backward.

In some examples, the input 1736x can include an actuator 1742x (e.g., button) in addition to or in lieu of the actuator 1740x. The actuator 1742x can be configured to enable the outer rigidizing device 2400/9300 to automatically shape copy the inner rigidizing device 2410/9310. The actuator 1742x can be similar to the button 1740x except that the button 1742x can be configured to be held down by the user until the desired advancement of the outer rigidizing device 2400/9300 has been reached. Upon release of the actuator 1742x, the outer rigidizing device 2400/9300 can be automatically rigidized and the inner rigidizing device 2410/9310 unrigidized. When the actuator 1742x is actuated, the shape copying sequence can automatically stop (e.g., as described with respect to button 1740x) if the user does not release the actuator 1742x before a signal to terminate advancement is detected by the system 2400z/9300z (e.g., the via alignment of the distal ends of inner and outer rigidizing devices 2410/9310, 2400, 9300, via a camera reading, or via a force sensor). The actuator 1742x can thus provide an automated shape copying sequence with more input by the user over the movement of the outer rigidizing device 2400/9300 than actuator 1740x. In some examples, the input 1736x can include an actuator 1745x (e.g., lever) in

addition to or in lieu of the actuators 1740x, 1742x. The actuator 1745x can be configured, when actuated, to switch modes of the actuator 1739x such that the actuator 1739x switches between enabling forward/backward motion of the inner rigidizing device 2410/9310 and forward/backward motion of the outer rigidizing device 2400/9300. In this example, when the user toggles between modes, the computational device 1737x and/or onboard electronics 1744x can automatically rigidize the former device (i.e., the device that is no longer controlled by the actuator 1739x) and unrigidize the current device (i.e., the device that is being controlled by the actuator 1739x).

In some examples, advancement of the outer rigidizing device 2400/9300 can be actuated automatically, such as when the inner rigidizing device 2410/9310 stops moving for longer than a set time interval or when the inner rigidizing device 2410/9310 cassette reaches a preset end of travel with respect to the outer rigidizing device 2400/9300.

In some examples, the system 2400z/9300z can include a mechanism designed to ensure that the outer rigidizing device 2400/9300 does not disturb the set shape (e.g., the shape of the steerable distal end section and/or the rigidized shape of the body) of the inner rigidizing device 2410/9310 as the outer rigidizing device 2400/9300 is advanced thereover. In one exemplary example, when the outer rigidizing device 2400/9300 is advanced, a tension sensor can be used by the computational device 1737x to enable adjustment to the position/orientation of the steerable distal end section of the inner rigidizing device 2410/9310. In an exemplary method of use, the tension on the steering cables (e.g., similar to cables 7624) can first be measured (i.e., after the inner rigidizing device 2410/9310 has been rigidized and before the outer rigidizing device 2400/9300 is advanced). The outer rigidizing device 2400/9300 can then be advanced over the inner rigidizing device 2410/9310, and the tension on the cables can be measured. The change in cable tension can be used (e.g., in conjunction with the stiffness of the cable) to determine the amount of unintended displacement of the steerable distal end section of the inner rigidizing device 2410/9310. After the unintended displacement has been determined, a counter displacement can be actuated by the cables to adjust the position of the distal end section of the inner rigidizing device 2410/9310 to another position. For instance, back to its starting position. In some examples, a sensor, such as a fiber optic sensor, may be used to sense the shape of the steerable distal end section of the inner rigidizing device 2410/9310 instead of or in addition to the tension sensor.

Any of the rigidizing members described herein may also include one or more sensors including optical sensors (e.g., cameras). For example, any of these rigidizing members may include a camera on the distal tip of the inner rigidizing device 2410/9310 that can be used in addition to or instead of the tension sensor to control movement (e.g., radial movement) of the inner rigidizing device 2410/9310 as the outer rigidizing device 2400/9300 is advanced thereover. That is, the camera on the inner rigidizing device 2410/9310 can be visually served in place to maintain a fixed position while the outer rigidizing device 2400/9300 is advanced thereover (i.e., the visual image from the camera of the inner rigidizing device 2410/9310 can be used as feedback to prevent the outer rigidizing device 2400/9300 from deforming the shape of the inner rigidizing device 2410/9310). In some examples, the computational device 1737x can prevent the outer rigidizing device 2400/9300 from advancing if the image displacement has reached a certain threshold, thereby preventing the outer rigidizing device 2400/9300 from having too much of an effect on the fixed shape of the inner rigidizing device 2410/9310. In other examples, the computational device 1373x can compute a vector based upon movement of the image and command the distal tip to move back to the original position (e.g., by adjusting the steering cables). Advantageously, using the camera to keep a fixed position of the distal tip of the inner rigidizing device 2410/9310 can also help ensure that the user is able to maintain a fixed visual position within the anatomy.

Similarly, in some examples, the system 2400z/9300z can include a mechanism designed to ensure that the inner rigidizing device 2410/9310 does not disturb the set shape (i.e., rigidized shape) of the outer rigidizing device 2400/9300 as the inner rigidizing device 2410/9310 advances therethrough. For example, the computational device 1737x can ensure that tension on the steering cables of the inner rigidizing device 2410/9310 is kept uniform at less than a threshold value (such as at a low value of less than ¼ lb) as the inner rigidizing device 2410/9310 advances, thereby ensuring that the inner rigidizing device 2410/9310 does not put too much force on the outer rigidizing device 2400/9300. In some examples, the tension in the cables can be limited to a certain (low) threshold value only until the steerable distal end section of the inner rigidizing device 2410/9310 is completely distal to the distal end of the outer rigidizing device 2400/9300. In some examples, the steering cables can be advanced while holding the tension they had immediately after the outer rigidizing device 2400/9300 was rigidized.

In some examples, shape copied curves may gradually straighten as they are propagated proximally along the system 2400z/9300z because a higher bending stiffness may lead to degraded shape copying fidelity, gap offsets, or the like. This gradual straightening (or “relaxation”) may be advantageous to assist in navigating through the tortuous anatomy (e.g., to reduce capstan drag). In one example, to encourage this straightening at the proximal end, the system 2400z/9300z can include a gradual taper in the diameter of the outer device 2400/9300 such that the device 2400/9300 (and system 2400z/9300z) includes a larger diameter at the proximal end and a smaller diameter at the distal end. By increasing the diameter at the proximal end, the system 2400z/9300z may be stiffer at the proximal end than the distal end and thereby encourage relaxation. In another example, to encourage this straightening at the proximal end, one or both of the inner rigidizing device 2410/9310 or the outer rigidizing device 2400/9300 can include a tapered space or gap in the device wall between the innermost layer 115 and the outermost layer 101 (see the exemplary wall layout in FIG. 3E). The gap can be larger at the proximal end of the respective device(s) 2410/9310, 2400/9300 and smaller at the distal end of the device(s) 2410/9310, 2400/9300. The smaller gap at the proximal end may result in a proximal end that is less flexible and therefore more likely to straighten.

In general, the flexibility and rigidity of the inner elongate member (“child”) and the outer elongate member (“parent”) may be matched so that the range of flexibility of each elongate member, which may be made as large as possible, is approximately the same and the first elongate member is approximately as flexible as the second elongate member when both are in the relaxed (not rigid) configuration.

In some examples, the system 2400z/9300z can be configured to enable commanded relaxation of the curvature of the shape copied formation while the system 2400z/9300z is within the anatomy. For example, it may be desirable to relax the curvature, i.e., partially straighten the rigidized state of both devices 2410/9310, 2400/9300, in order to reduce capstan drag as the devices 2410/9310, 2400/9300 are slid relative to one another and/or as a third device is passed through the system 2400z/9300z. Advantageously, the curvature relaxation can be configured so as to slightly pull or straighten the anatomy (e.g., the colon) within which the system 2400z/9300z is positioned without causing harm thereto.

In one exemplary example of commanded curvature relaxation, both the inner rigidizing device 2410/9310 and the outer rigidizing device 2400/9300 can be relaxed simultaneously, enabling the entire system 2400z/9300z to tend towards straightness (though constrained partially by the anatomy). In this example, the combined stiffnesses of the inner and outer rigidizing devices 2410/9310, 2400/9300 in the flexible configuration can be designed to be less than the stiffness of a device known to be safe in the anatomy. For example, for use in the colon, system 2400z/9300z can have a combined stiffness that is less than that, for example, of a standard adult colonoscope, thereby ensuring that the system 2400z/9300z will exert no more force on the colon during curvature relaxation than a device known to be safe.

In another exemplary example of commanded curvature relaxation, the inner rigidizing device 2410/9310 and the outer rigidizing device 2400/9300 can be alternately rigidized and unrigidized without translating either device 2410/9310, 2400/9300, thus resulting in a gradual relaxation of the curves (as each copying cycle may inevitably allow the system 2400z/9300z to straighten slightly). For example, both devices 2410/9310, 2400/9300, can first be rigidized. A first of the inner or outer rigidizing devices 2410/9310, 2400/9300 can then be relaxed and subsequently rigidized. The second of the inner or outer rigidizing devices 2410/9310, 2400/9300 can then be relaxed and subsequently rigidized. The loop of rigidization/relaxation can be repeated until the shape of the system 2400z/9300z is smoothed (i.e., the curvature relaxed) to the desired shape.

In another exemplary example of commanded curvature relaxation, a combination of the above two mechanisms can be used to phase the transfer between the rigidized and unrigidized states. That is, the system 2400z/9300z can fluctuate between having both devices 2410/9310, 2400/9300 rigidized to having only one device 2410/9310, 2400/9300 rigidized to having neither device 2410/9310, 2400/9300 rigidized, to having only one device 2410/9310, 2400/9300 rigidized, to rigidizing both devices 2410/9310, 2400/9300, etc.

Any of the methods of commanded curvature relaxation described herein can be modified to adjust the amount of relaxation desired. For example, the number of cycles (of rigidizing and unrigidizing) can be varied to change the amount of relaxation. As another example, the frequency and/or duty cycles of the cycles can be varied (e.g., how long the unrigidized configuration is maintained). As another example, the pressure and/or vacuum applied during the rigidized and unrigidized phases can be modified (e.g., instead of releasing all of the vacuum/pressure in the unrigidized phase, a partial release can be performed to allow the braid to slip only in the highest curvature regions).

In some examples of commanded curvature relaxation, the relaxation can be applied up to a particular threshold. For example, the relaxation sequence can be performed until only bends tighter than a set radius (e.g., a radius of 2 inches) are relaxed.

In some examples of commanded curvature relaxation, the steerable distal end section of the inner rigidizing device 2410/9310 can be used to selectively modify the shape of the outer rigidizing device 2400/9300 (e.g., to relax the curvature in a specific location).

In some examples, commanded curvature relaxation can be used to assist in passing working tools through the system 2400z/9300z. For example, the system 9300z can be relaxed until the working tool passes through the system 2400z/9300z to reduce the tortuosity that the tool is exposed to.

In some examples, the system 2400z/9300z can be used to perform a reduction maneuver within the anatomy (i.e., to straighten a tortuous path within the anatomy, such as within the colon). In some examples of a reduction maneuver, the distal end of the system 2400z/9300z can be anchored (e.g., by angulating around a bend in the colon, such as the splenic flexure), and then the entire system 2400z/9300z (i.e., both the inner and outer rigidizing devices 2410/9310, 2400/9300) can be pulled proximally while in the flexible configuration. Pulling proximally on the system 2400z/9300z while the distal end is anchored can straighten the lumen distal to the anchoring point, thereby straightening the tortuous path.

In some examples, the system 2400z/9300z can be used to unfurl or unloop the anatomy during insertion and/or as part of a reduction. For example, the system 2400z/9300z can be used to enter the patient's colon to the patient right and form an alpha loop to open up a folded sigmoid colon.

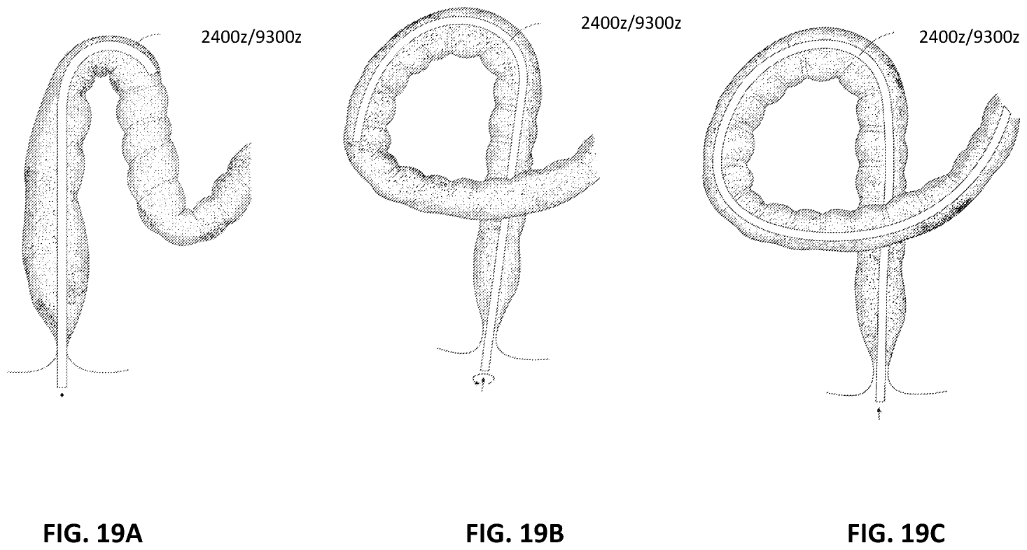

In some examples, the system 2400z/9300z can be used to perform a maneuver similar to twisting the entire system 2400z/9300z from the proximal end (e.g., to assist in unfurling or unlooping the anatomy). That is, the maneuver can enable the system 2400z/9300z to mimic the movement that would occur if the system 2400z/9300z were bent at a fixed angle and then the entire system was rotated (e.g., by 90-180 degrees) from the proximal end while maintaining fixed angle of the bend. FIGS. 19A-19C illustrate an example of a twisting maneuver that can enable unfurling of a curved loop, such as of an N-loop. That is, as shown in FIG. 19A, the system 2400z/9300z can be advanced into the colon up until the first curve of the N-loop. At FIG. 19B, the system 2400z/9300z can activate a twisting maneuver (i.e., to mimic twisting) and be advanced further into the colon, thereby pulling and looping the colon over itself and enabling navigation through a larger radius of curvature rather than the tight radius of curvature in the N-loop. The system 2400z/9300z can then be advanced further into the colon through the large radius curve. Although shown as being used in the colon to straighten an N-loop, in this example, it should be understood that the methods and apparatuses for reducing a portion of the anatomy as described herein (including but not limited to the twisting maneuver) may be used in other anatomical locations.

In one example, the twisting maneuver can be performed by activating a plurality of the cables on the inner rigidizing device 2410/9310 simultaneously. For example, in some examples, the inner rigidizing device 2410/9310 can include a steerable distal end that is controlled by actuating four cables (two cables to control the actuation in the “x-plane” and two cables to control the actuation in the “y-plane”). The “x-plane” (or roll) can be controlled by moving the actuator 1738x left-right while the “y-plane” (or pitch) can be controlled by moving the actuator 1738x up-down. Moving the actuator 1738x up can pull the y-positive cable taught and tilt the inner rigidizing device 2410/9310 “north.” When the twisting maneuver is actuated (e.g., by pressing an actuator, such as a button, on the input 1736x), the motion of the actuator 1738x and x, y output can be no longer correlated in the same way. Rather, when a bend is formed and the twisting maneuver is actuated, the computational device 1737x (e.g., a controller) can calculate the polar coordinates radius r and angle θ using the equations below:

r=\sqrt{x²+y²} and θ=atan 2(x,y)

Once those values are calculated, a unique cosine and sine curve can be calculated that will serve as the calculator to output the new x and y coordinates, where r serves as the amplitude for the y axis and e is the initial location along the x-axis between intervals (−180°, 180°]. In this twisting maneuver, the actuator 1738x may simply require one axis (e) to output both the x and y values necessary to actuate the bending section. The computational device 1737x can cycle through the range (−180, 180] and output x and y using the equations below:

x=r cos(θ) and y=r sin(θ)

In this example, movement of the actuator 1738x left or right can cause the computational device 1737x to cycle through a value θ up or down between a range of (−180 and 180] as described above, which can result in a computation of the x and y values for movements of the cables. Moving the actuator 1738x left-right can therefore result in rotating or twisting the bending section (i.e., rotating/twisting clockwise or counterclockwise).

In some examples, before the twisting maneuver, the tip of the outer rigidizing device 2400/9300 can be advanced distal to the tip of the inner rigidizing device 2410/9310 to extend the length of the arc created.

In some examples, the system 2400z/9300z can be configured to maneuver into a tighter curve than the steerable distal end section of the inner rigidizing device 2410/9310 can achieve on its own. For example, the steerable distal end section of the inner rigidizing device 2410 can be positioned such that it is only partially covered by the outer rigidizing device 2400/9310. If the rigidizing device 2400/9300 is placed in the rigid configuration (with the steerable distal end section of the inner rigidizing device 2410 partially covered), the high stiffness of the outer rigidizing device 2400/9300 can prevent the covered portion of the steerable distal end section of the inner rigidizing device 2410/9310 from bending. As a result, only the exposed portion of the distal end section will bend, enabling the formation of a tight (i.e., low radius of curvature) and small bend or curve in the distal end section.

In some examples, the system 2400z/9300z can be used to map the shape of the anatomy (e.g., the colon) through which the system 2400z/9300z passes. For example, after placement and rigidization of the outer rigidizing device 2400/9300, the inner rigidizing device 2410/9310 can be withdrawn in a flexible state into the outer rigidizing device 2400/9300. As the device 2410/9310 is withdrawn (and/or as it is inserted again after withdrawal) within the rigid outer device 2400/9300, the tension on the steering cables can be monitored (e.g., with a tension sensor and computational device 1737x as described herein) to map the shape of the rigidized outer device 2400/9300 (and thus, correspondingly, the shape of the anatomy). As another example, the outer rigidizing device 2400/9300 can be withdrawn and then reinserted. As the device 2400/9300 (in the flexible configuration) is reinserted over the rigid inner device 2410/9310, a force sensor can be used to measure the insertion forces of the outer rigidizing device 2400/9300 to measure the severity of the bends of the inner rigidizing device 2410/9310 (and thus, correspondingly, the shape of the anatomy).

Similarly, in some examples, with knowledge of the commanded distal articulations of the steerable distal end of the inner rigidizing device 2410/9310 and the subsequent shape copying sequence (including axial motion of the inner and outer devices 2410/9310, 2400/9300), an estimate of the accumulated overall shape of the system 2400z/9300z can be produced and used for control, visualization, and/or general situational awareness. Additionally, the efficiency and/or accuracy of shape copying can be modeled either empirically or analytically and applied to the overall shape estimate. Alternatively, in some examples a shape sensor may be used to estimate the shape without visualization and/or in some cases with visualization.

In some examples, a camera on the distal end section of the inner rigidizing device 2410/9310 can be used as part of a computer vision algorithm (e.g. by the computational device 1737x) for steering and/or control of the system 2400z/9300z. For example, the computational device 1737x can use a computer vision algorithm to determine when the steerable distal end section of the inner rigidizing device 2410/9310 is moving. The computational device 1737x can then calculate the net rotational and linear motion of the camera from the motion of elements in a camera view at a known or approximate range. This can be useful, for example, to detect how a motion of a steering cable affects the steering of the steerable distal end section to ensure calibration of the steering. As another example, the computational device 1737x can use a computer vision algorithm to enable an automatic lumen following mode (i.e., via detection of the lumen with the computer vision algorithm). In this example, for example, the user (e.g., physician) can command the insertion axis for the system 2400z/9300z, and the system 2400z/9300z can automatically articulate following the commanded path. In another example, the system 2400z/9300z can provide the user with a visual guide suggesting where to drive the inner rigidizing device 2410/9310 for optimal lumen following. In this example, the visual guide can be rendered on the endoscope view as a target for the physician to aim the camera. In another example, the system 2400z/9300z can provide the user with steering guides (e.g. a potential field) that bias the driving commands for the inner rigidizing device 2410/9310 towards the optimal trajectory while still allowing the physician to “push through” (i.e., ignore) the guides if desired.

In some examples, the system 2400z/9300z can be configured to undergo a diagnostic mode. For example, in diagnostic mode, the inner rigidizing device 2410/9310 can be partially or fully withdrawn into the outer rigidizing device 2400/9310 (and/or the outer rigidizing device 2400/9300 can be moved partially thereover). If it is suspected that a steering cable on the inner rigidizing device 2410/9310 is broken, then a diagnostic test can be performed of the steering cables and their respective actuators. For example, a motor controlling a steering cable of the inner rigidizing device 2410/9310 can be actuated such that the distal bending section of the inner rigidizing device 2410/9310 presses against the inside of the rigidized outer device 2400/9300. During this maneuver, a corresponding increase in tension on the steering cables should be seen. If there is no increase in tension, then it can be determined that the respective steering cable may be broken.

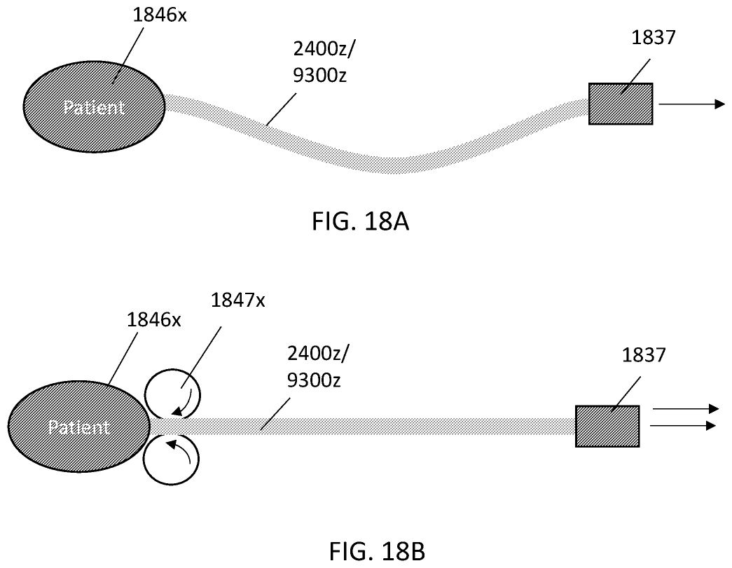

Referring to FIG. 18A, in some examples, when the system 2400z/9300z is being withdrawn in the entirely flexible configuration (e.g., for removal or for a reduction maneuver), the system 2400z/9300z may sag outside of the patient's body 1846x. To compensate for this sagging, a support and/or anti-buckling device can be used to support the system 2400z/9300z outside of the body. In other examples, referring to FIG. 18B, to compensate for sagging, the system 2400z/9300z can include a plurality of drive wheels 1847x or other gripping mechanism at the entrance point (e.g., mouth or anus) to the body lumen through which the system 2400z/9300z is maneuvering. The drive wheels 1847x can advantageously maintain tension on the system 2400z/9300z as it is withdrawn, thereby keeping the portion of the system 2400z/9300z that is external to the body 1846x relatively straight and self-supporting from the body 1846x to the proximal termination point (e.g., cassette 1837). In other examples, the system 2400z/9300z can be used without an anti-buckling device and/or other mechanism to support the system 2400z outside of the body by not transitioning the system 9400z to a fully flexible configuration. That is, the system 2400z can be used and configured such that at least one device 2400, 2410 is always rigidized (i.e., as the devices 2410, 2400 are alternately advanced and rigidized). As such, the majority of the length of the system 2400z overall can be rigid during use. Due to its rigid state, the system 2400z can be stable outside of the body (i.e., during insertion) without an external anti-buckling device and/or support mechanism.

In some examples, whenever the inner rigidizing device 2410/9310 is moving forward, the position of the steering cables can be precisely controlled. In contrast, whenever the inner rigidizing device 2410/2400 is moving backwards or the outer rigidizing device 2400 is moving forwards, the tension on the cables can be precisely controlled.

It should be understood that where “tension” is described with respect to the steering cables herein (e.g., monitoring tension), other mechanisms for actuating the cables are possible (e.g., hydraulic pressure).

In some examples, the inner rigidizing device and the outer rigidizing device may be advanced by the robotic system described herein using small steps (e.g., less than 1-inch steps). Small steps may advantageously allow for more precise control of the placement and orientation of the rigidizing devices. For example, the user may steer the inner tube in the desired direction and, as the inner tube advances ahead of the outer tube by a small amount (for instance, ½, ¾ or just under 1 inch), the sequence of rigidization and advancement or retraction of the outer tube can be triggered automatically. In some examples, the present sequence of small steps can be overridden when desired. In some examples, the inner rigidizing device and outer rigidizing device may be advanced by the robotic system using medium steps (e.g., 1-3 inch steps) or large steps (e.g., greater than 3 inch steps).

The cassettes and/or tools described herein may be disposable or reusable or used and cleaned for a limited number of cycles.

The linear slides described herein can, in some examples, be U-shaped with a corresponding U-shaped tract. Alternatively, the linear slides can, in some examples, be circular with a corresponding circular shaped tract.

In some examples, the tip of the outer rigidizing device can include one or more cameras to view the end effector of the tool used with a robotic system. This can allow a controller of the robotic system to calculate the relation between the control inputs and effector outputs and adjust accordingly to give the same effector motion regardless of the tooth path (e.g., regardless of drag placed on the tool control cables during bending).

Although the outer rigidizing device for the nested systems described herein is often referred to as rigidizing via vacuum and the inner scope rigidizing device as rigidizing via pressure, the opposite can be true (i.e., the outer rigidizing device can rigidize via pressure and the inner rigidizing device via vacuum) and/or both can have the same rigidizing source (pressure and/or vacuum).

Although the inner and outer elements of the nested systems are generally described as including integrated rigidizing elements, the rigidizing elements can be separate (e.g., so as to allow relative sliding between the imaging scope elements and the rigidizing elements).

The rigidizing devices of the nested systems described herein can be designed such that inner rigidizing device can't rotate substantially within outer rigidizing device when they are assembled. For instance, the outer surface of the inner rigidizing device can have longitudinal ridges and grooves that form a spline. The inner surface of the outer rigidizing device can have corresponding ridges and grooves that mate with the same features in the outer rigidizing device.

Either or both of the rigidizing devices of the nested systems described herein can be steerable. If both rigidizing devices are steerable, an algorithm can be implemented that steers whichever rigidizing device is flexible and moving longitudinally. The algorithm can steer the flexible rigidizing device to anticipate the shape of the rigidized device thus minimizing the tendency for the moving, flexible rigidizing device to straighten the rigid device.

If one rigidizing device of the nested systems described herein requires vacuum and the other rigidizing device requires pressure, user controls can be constructed in which moving one vs. the other (outer and inner) involves flipping a switch, with the switch toggling between a first condition in which, for example, one is pressurized for rigidity when the other is vented for flexibility and a second condition in which one is vented for flexibility and the other is vacuumed for stiffness. This, for example, could be a foot pedal or a hand switch.

In some examples, the alternate movement of the nested systems described herein can be controlled manually. In other examples, the alternate movement can be controlled automatically, via a computer and/or with a motorized motion control system.

The nested systems described herein can advantageously be of similar stiffness. This can ensure that the total stiffnesses of the nested system is relatively continuous. The nested systems described herein can be small so as to fit in a variety of different anatomies. For example, for neurology applications, the outside diameter of the system can be between 0.05″-0.15″, such as approximately 0.1″. For cardiology applications, the outside diameter of the system can be between 0.1″-0.3″, such as approximately 0.2″. For gastrointestinal applications, the outside diameter of the system can be between 0.3″-1.0″, such as 0.8″. Further, the nested systems described herein can maintain high stiffness even at a small profile. For example, the change in relative stiffness from the flexible configuration to the rigid configuration can be multiples of 10x, 20x, 30x, and even larger. Additionally, the nested systems described herein can advantageously move smoothly relative to one another.

The nested systems described herein can advantageously navigate an arbitrary path, or an open, complex, or tortuous space, and create a range of free-standing complex shapes. The nested systems can further advantageously provide shape propagation, allowing for shape memory to be imparted from one element to another. In some examples, periodically, both tubes can be placed in a partially or fully flexible state such that, for instance, the radii or curvature of the system increases, and the surrounding anatomy provides support to the system. The pressure or vacuum being used to rigidize the tubes can be reduced or stopped to place the tubes in a partially or fully flexible state. This momentary relaxation (for instance, for 1-10 seconds) may allow the system to find a shape that more closely matches the anatomy it is travelling through. For instance, in the colon, this relaxation may gently open tight turns in the anatomy.

In some examples, the stiffness capabilities of the inner or outer rigidizing devices may be designed such that tight turns formed by the inner rigidizing device at its tip, when copied by the outer rigidizing device, are gradually opened up (made to have a larger radius) as the shape propagates proximally down the outer tube. For instance, the outer rigidizing device may be designed to have a higher minimum radius of curvature when rigidized.

The nested systems are continuous (i.e., non-segmented) and therefor provide smooth and continuous movement through the body (e.g., the intestines). The nested systems can be disposable and low-cost.

In some examples, the outer rigidizing device can be a dynamically rigidizing overtube (e.g., as described in PCT/US18/42946, the entirety of which is incorporated by reference herein). In some examples, the inner rigidizing device can be a rigidizing system or a commercially available scope, for example a 5 mm diameter nasal scope. Utilizing rigidization and a nested system enables the utilization of a smaller scope that delivers, compared to a duodenoscope, more flexibility if desired, more stiffness if desired, enhanced maneuverability, and the ability to articulate at a much smaller radius of curvature.

In some examples, upon reaching the target destination, the inner rigidizing device of a nested system can be withdrawn. The outer rigidizing device can remain rigidized, and contrast can be injected through the inner element's space to fluoroscopically image.

RF coils can be used in any of the nested systems described herein to provide a 3-D representation of whatever shape the nested system takes. That representation can be used to re-create a shape or return to a given point (e.g., for reexamination by the doctor after an automated colonoscopy).

In some examples, the nested systems described herein can be useful as a complete endoscope, with the internal structure carrying the payload of working channels, pressurization lines, vacuum lines, tip wash, and electronics for lighting and imaging (vision systems, ultrasound, x-ray, MRI).

The nested systems described herein can be used, for example, for colonoscopy. Such a colonoscopy nested system can reduce or eliminate looping. It could eliminate the need for endoscopic reduction. Without looping, the procedure can combine the speed and low cost of a sigmoidoscopy with the efficacy of a colonoscopy. Additionally, colonoscopy nested systems can eliminate conscious sedation and its associated costs, time, risks, and facility requirements. Further, procedural skill can be markedly reduced for such colonoscopy procedures by using the nested systems described herein. Further, in some examples, the nested systems described herein can provide automated colonoscopy, wherein a vision system automatically drives the nested system down the center of the colon while looking for polyps. Such an automated system would advantageously not require sedation nor a doctor for the basic exam while allowing the doctor to follow up for further examination if required.

As described above, in some examples, the system (e.g., system 2400z/9300z) can be used to reduce the curvature through a body lumen, such as the colon. This action comprises a reduction of the body lumen. During traditional endoscopy of a body lumen (e.g., colonoscopy), if a loop has been formed and there is a tight turn within the loop, a clinician will generally attempt to perform a reduction. To reduce the loop, the clinician will torque consistently in the direction of least resistance.

For example, when performing a reduction of an alpha loop, a clinician may hook the bending section of the scope or catheter around a tight turn. The clinician then torques the scope in the direction of least resistance (normally counter-clockwise). Torquing the scope in this way “lifts” the loop and increases the “moment arm” to reduce loop. The clinician may then manually try to retract the scope, while torquing the scope the entire time. Ideally, this should be performed only as long as there is little or no resistance during these motions. The clinician may actuate the distal end of the bending section to maintain lumen-centricity during a reduction. Once reduced, the clinician removes the loop outside the patient, and continues to advance carefully. This process may be unwieldy and difficult to perform with traditional endoscopes, as it is difficult to coordinate the movement of the distal, intermediate and proximal regions in a manner that avoids harming the patient. It may also be particularly difficult to maintain the reduction once performed, to allow the apparatus to be further inserted/withdrawn, oriented (rotated) or used to transmit tools along a working channel.

The rigidizing apparatuses described herein may be particularly useful and helpful for preforming simple, safe and reliable reductions, e.g., of the colon. The methods and apparatuses described herein may provide a simple technique that minimize complicated maneuvers and may reliably minimize slipping of the distal end of the apparatus. These methods and apparatuses may be configured to prevent the colon from getting caught up and twisted, including in examples in which the rigidizing apparatus may form a hook at a distal end to reduce the colon.

When performing colonoscopies, looping is a frequent challenge, and may occur when the colonoscope stretches and distends the colon in response to advancing of the scope forward (distally). Typically, once a loop has formed, it must be straightened (“reduced”) before the procedure can continue. However, it may be particularly challenging to reduce a loop with nested colonoscopes. The methods and apparatuses described herein may provide, for the first time, reduction of loops by controlling both the rigidity and relative position (axial and/or rotational) of nested elongate members. Advantageously, some or all of these techniques may be automated or semi-automated.

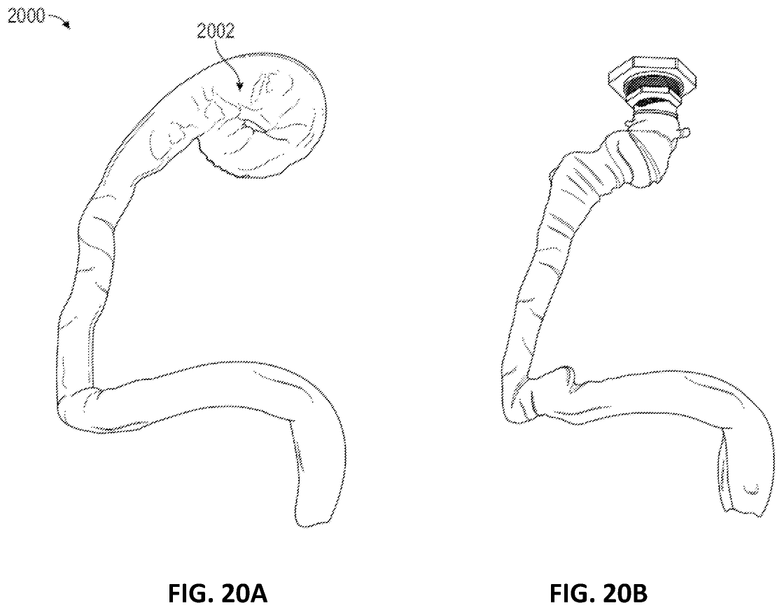

For example, FIGS. 20A and 20B show an exemplary reduction of an alpha loop 2002 formed in a model of a colon 2000. In FIG. 20A the colon model includes an alpha loop 2002 in which the colon, and the nested rigidizing members positioned within the colon, for a nearly 360 degree loop. As will be described in greater detail below, a reduction technique using the selectively rigidizing apparatus can be employed to reduce the loop, resulting in the less curved colon shape as shown in the model colon in FIG. 20B. The apparatuses and methods described herein may use a variety of features associated with the selectively rigidizing apparatus in order to perform reductions as described herein. These features are described in greater detail below and may include a floating (rotational unconstrained) proximal (e.g., “handle”) region. Any of the apparatuses described herein may use two or more nested (e.g., telescoping) tubes in which each of the tubes (or a tube and an inner member) are selectively rigidizing. The proximal end region of some or all of the telescoping members may be configured to freely rotate, for example, at the proximal clamp region. In some examples, the apparatus includes a rigidizing outer (“mother”) member and a separately rigidizing inner (“child”) member; either or both the outer member and inner member may be configured to freely rotate, or float. In some examples just the inner member or just the outer member may be freely rotated. The apparatus may be configured so that rotation at the proximal end may be engaged automatically or manually, allowing the apparatus to transition from a rotationally locked (or controlled) configuration to a freely rotating configuration.

Any of these apparatuses may be configured so that the rigidizing member (or a plurality of telescoping members) may be actively rotated or driven in rotation by the apparatus. In examples in which two telescoping member (inner and outer) are included, either or both the inner and outer members may be configured to be driven in rotation from the proximal end region.

As describe above, the apparatus may be configured to perform one or more preset motions that may be used for reductions. For example, a “spin move” may be preset or programmed into the apparatus and may be triggered manually or automatically. Manually triggering the spin move may allow the system to perform a predetermined series of movements (e.g., of the steering subsystem for controlling a distal end region of the apparatus, e.g. by operating tendons as described above) so that the distal end region forms a hooking or curved shape in a plane; the apparatus may maintain this curved or hooked configuration of the distal end region in the same plane relative to the colon while rotating the more proximal region or allowing the more proximal region to rotate out of this plane. For example, the apparatus may be configured so that the distal end of the apparatus, or in some examples the distal end region of the inner (e.g., child) member may be steered into a curve (hook, or J-shape), and the system may be configured to maintain this articulation angle while manually or automatically changing the articulation plane. This may be achieved by actively steering the distal end region using the steering member (e.g., tendons). The apparatus may be configured to robotically perform these techniques.

Any of the apparatuses described herein may also or alternatively be configured to provide non-uniform bending stiffness radially along the elongate stiffening member long axis. In an example of an apparatus including a pair of stiffening members (e.g., an inner and an outer member), one or both of the members may be configured to be highly flexible in a first plane oriented along the long axis; for example, the apparatus may be highly flexible in the 0 and 180 degree radial positions, but stiffer (e.g., by 1.5×, 2×, 2.5×, 3×, etc.) in a plane that is perpendicular to this, e.g., in the 90 and 270 radial positions. In some examples where multiple telescoping members are included, the relatively stiff and relatively more flexible radial positions may be radially offset relative to each other.

Any of the apparatuses described herein may be partially rigidizing. For example the amount of rigidization may be adjustable or selectable by adjusting or selecting the suction (in some examples) or positive pressure (in other examples) applied. For example, increasing the positive pressure may result in an increase in the rigidity along the entire length. In any of these examples the apparatus may be configured with telescoping members that are partially rigidizing. This may allow the apparatus to achieve variable nested plastic deformation.

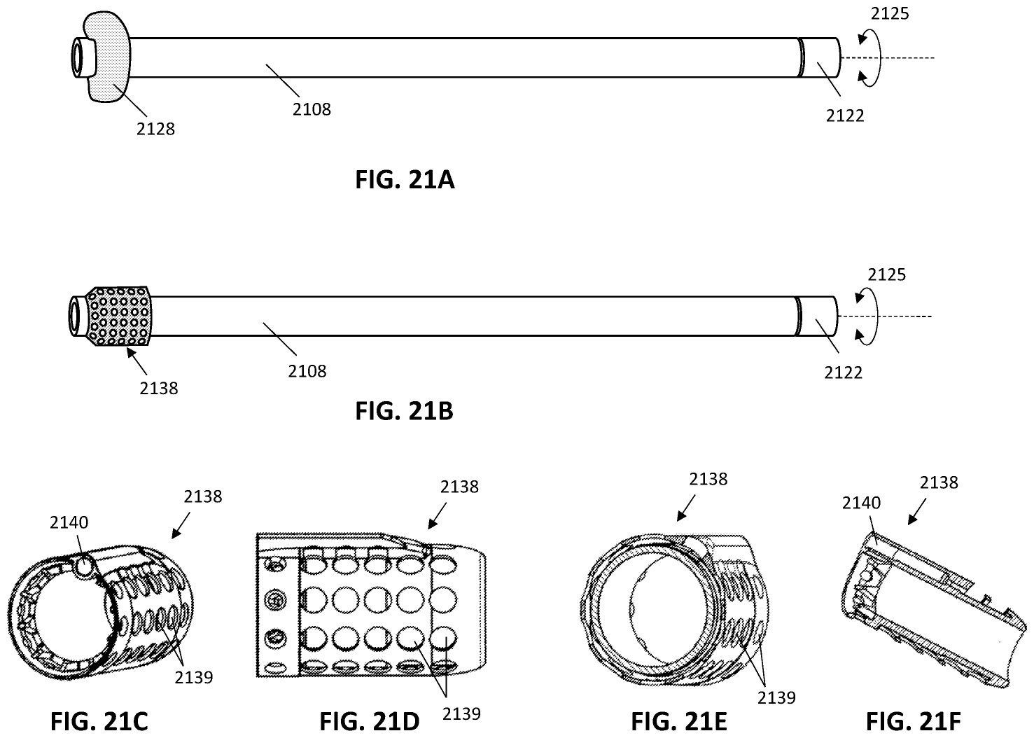

The apparatuses described herein may alternatively or additionally include an anchor, such as a balloon, at the distal end region of the apparatus; in variations including multiple, telescoping members one or more balloons may be used. For example, in a telescoping apparatus the outer member may be configured to expand (e.g., as an inflatable balloon or other expanding structure). In some examples the distal end region of an inner member may be configured to expand (e.g., a balloon). Thus, in any of these apparatuses and methods an anchor such as a balloon may be used to reduce. For example, FIG. 21A illustrates one example of an elongate rigidizable member 2108 that may be, e.g., an outer 2400 or an inner 2410 elongate rigidizable member that includes a balloon 2128 at the distal end, which may be coupled to an inflation lumen (not shown) running on an outside of the member, an inside of the member or between the walls of the member. The anchoring balloon 2128 may be positioned at the distal end of the elongate rigidizable member or more proximally. Although a single donut-shaped (e.g., toroidal) balloon is shown, multiple balloons may be used and arranged radially around, and/or in some cases longitudinally along, the elongate member. Balloons may be configured to be inflated with any appropriate inflation material, including saline.

The apparatuses and methods described herein may also or alternatively include a suction through a suction chuck or opening (or multiple openings) to apply suction to hold or grasp the wall of the colon lumen. This may allow the apparatus to secure to a portion of the colon wall in a controllable (including robotically controllable manner). For example, FIG. 21B illustrates an example of an elongate rigidizable member 2108 that includes a vacuum port (or vacuum chuck) 2138 at the distal end region of the elongate rigidizable member.

FIGS. 21C-21F show alternative views of a vacuum port 2138 that may be used. In FIG. 21C the vacuum port 2138 includes multiple openings 2139 through which vacuum may be delivered. The vacuum port also includes a connection 2140 to a vacuum (suction) line that may provide suction to the ports. In this example a large number of small openings 2139 may be included to allow radial coupling to the walls of the colon. A vacuum line (not shown) extending from the proximal end may be coupled to the vacuum port 2318. FIGS. 21C and 21E show proximal perspective view and distal perspective views, respectively. FIG. 21D shows a side view and FIG. 21F shows a section through the vacuum port

Alternatively in some examples the apparatus may be configured to that the rigidity may be varied along the length of the apparatus. For example, these apparatuses may include rigidization zones along the length of the member that allows the apparatus to rigidize a portion (e.g., the distal 10 cm) rather than the entire length. This may be achieved by including separately addressable and/or multiplexed pressure/suction regions within the length of the apparatus. The apparatus (e.g., a controller) may be used to apply positive pressure (or in some examples suction) to rigidize just one region, or multiple (e.g., all) regions. Where multiple regions are included, different pressure levels and therefore different rigidities may be achieved.

Any of the apparatuses described herein may include shape sensing in which the apparatus may determine the shape assumed by the elongate member. For example a fiber optic shape sending element may be included along all or portion of the length of the apparatus and may be used to determine the shape of the elongate rigidizable member (or members) forming the apparatus. Fiber Optic Shape Sensors (FOSS) may be used and may be based on optical multicore fibers (MCF) or multiple optical single-core fibers with embedded strain sensors. Shape sensing may be used to allow a user to see the shape of the current system and the controller (e.g., processor controlling and/or coupled to the apparatus) to interpret the path taken by the robotic apparatus. This may also be used to assist the apparatus in performing the reductions described herein. Any of the methods and apparatuses described herein may also or alternatively be used with shape estimation based upon history of individual articulations, movements, and copying of shapes between mother and child, enabling some of the same additional features and algorithms enabled by shape sensing.

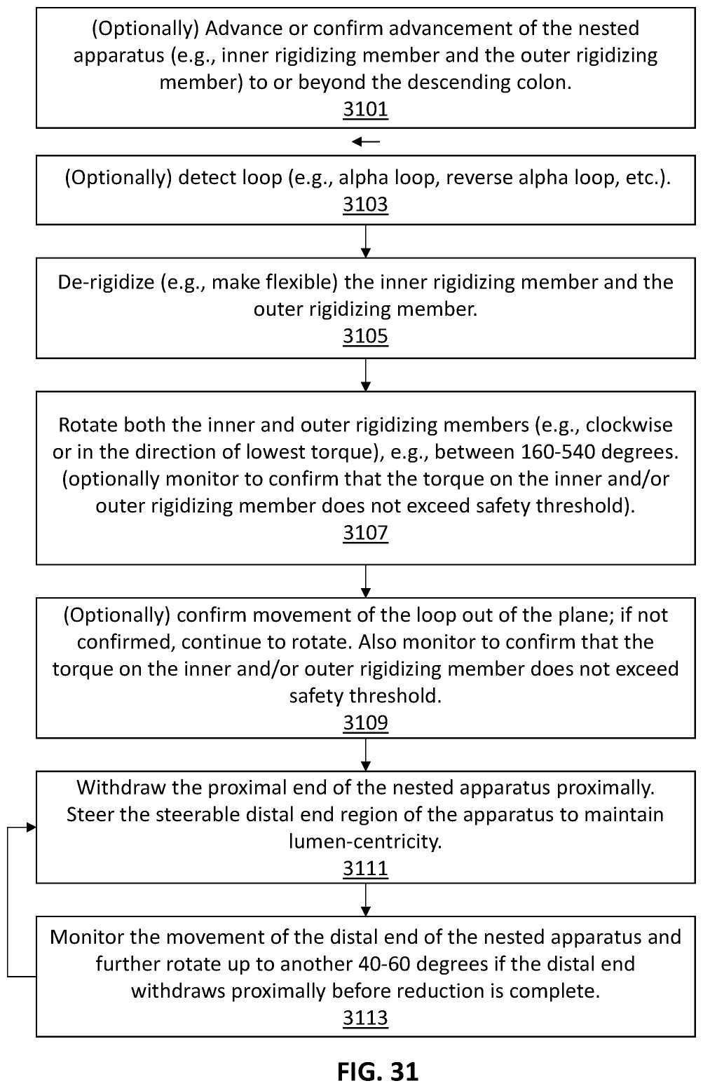

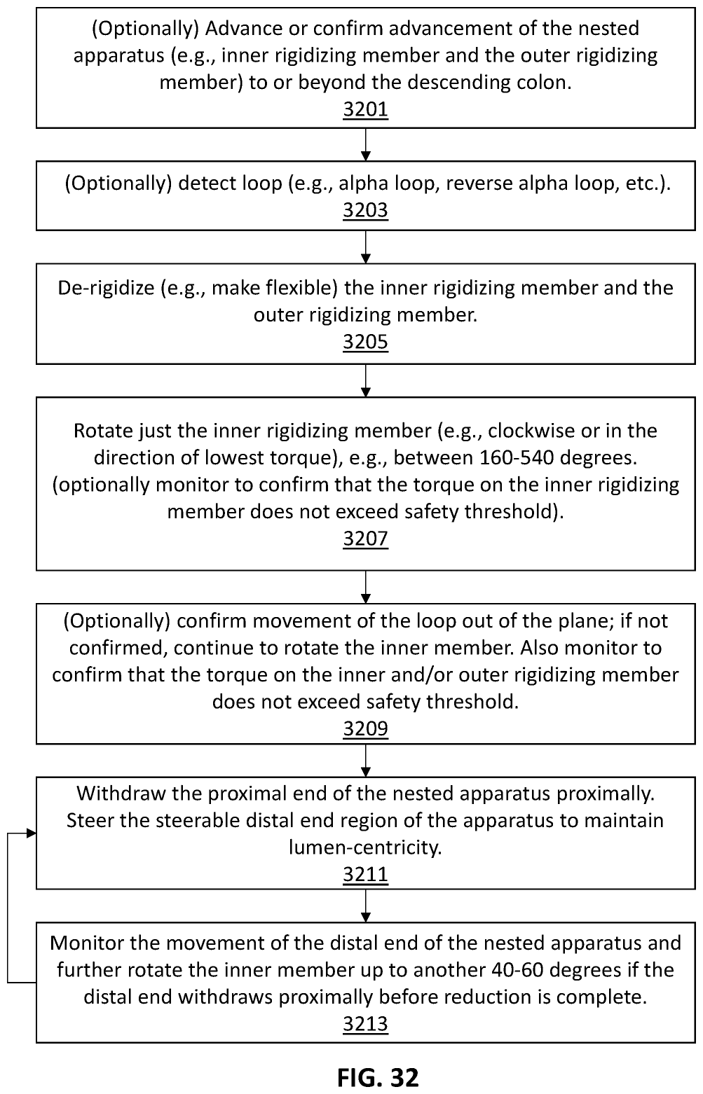

In general, the apparatuses and methods described herein may use the shape of the nested inner and outer rigidizing members when in the body lumen to control or assist in performing reductions, e.g., automatically or semi-automatically as described herein. For example, in any of these methods and apparatuses, a display may show the actual or an estimated shape of the length of the nested inner and outer rigidizing members. Thus the user may see which type of reduction or set of reduction maneuvers may be selected or used to perform a successful reduction. Alternatively the apparatus may use the estimated or determined shape of the nested inner and outer rigidizing members to select or suggest the reduction maneuver(s) to be applied to reduce a loop. For example, if the nested inner and outer rigidizing members forms an alpha loop, the apparatus may suggest or perform the steps of: making the nested inner and outer rigidizing members flexible, rolling either both the nested inner and outer rigidizing members (or just the inner rigidizing member), and withdrawing the proximal end of the nested inner and outer rigidizing members, as described below in FIGS. 31-32. In some examples if the nested inner and outer rigidizing members forms an N-loop, the apparatus may suggest or perform the steps of: making the nested inner and outer rigidizing members flexible, and withdrawing the proximal end of the nested inner and outer rigidizing members, as described below (e.g., without rolling the nested inner and outer rigidizing members).

The method and apparatuses described herein may also be used with force/torque feedback. For example, any of the apparatuses described herein may include force/torque feedback from a bending section of the elongate member (e.g., the inner member) and/or in both the outer member and inner member handles in variations including an inner member and an outer member.

Other sensing and/or feedback may be used as well for any of the methods and apparatuses for reducing curvature (e.g., robotically reducing curvature) as described herein. For example, any of these methods and apparatuses may be used with visual feedback. For example, visual feedback may be used from a camera view that allows the apparatus (e.g., the controller of the apparatus) to interpret the lumen, and/or position and/or to maintain view.

Any of these methods and apparatuses may also or alternatively include the use of machine learning, or a machine learning agent (e.g., artificial intelligence) to identify patterns in movement and/or articulation during a colonoscopy. For example, a system may be trained using multiple colonoscopy cases, and these cases may be marked to indicate successful reductions. This training may be updated continuously or periodically and may allow the machine learning agent to identify, and either perform or suggest performance of one or more maneuvers, including those described herein, in order to reduce the colon.

The apparatuses described herein may generally be referred to as robotic apparatuses that may include a controller to perform one or more of the maneuvers described herein either triggered manually or automatically (or semi-automatically). For example, any of these apparatuses or methods include presenting the user with a control or command (e.g., a virtual or actual control, e.g., button, that a user may activate to perform a coordinated series of actions in order to reduce and/or maintain reduction. Alternatively or additionally the apparatus or method may be configured to automatically determine when to perform the reduction maneuver and may perform it when the system detects it would be best performed, e.g., using a machine learning agent or AI.

One of technique that may be used by any of the methods and apparatuses described herein may include allowing free rotation (“floating”) of any of the elongate members (e.g., inner elongate member, outer elongate member) at the handle region, while forming a distal hook or end at the steerable distal end and pulling the elongate member(s) in a flexible configuration. This maneuver may be referred to herein as a floating handle maneuver and may be used alone or in combination with other maneuvers. The apparatus may be configured to perform the maneuver, including having a handle that may be freely rotatable or may transition to a freely rotatable configuration. The apparatus may also be configured to form a hook at the steerable distal end region of a rigidizable elongate member of the apparatus (e.g., in some examples a rigidizable inner member). The apparatus and method may also be configured to include a control that allows the user to select this (and/or other predetermined techniques, including reduction techniques including those described herein).

Another technique that may be used includes rotating (“spinning”) one or more of the rigidizable members (e.g., in examples having telescoping rigidizable members) of the apparatus, while or after forming a hook or bend at the steerable distal end and pulling the elongate member(s) and pulling proximally on the elongate member(s). The apparatus or method may maintain the hook or bend at the distal end in the same plane relative to the body as the elongate member proximal region rotates. This may be referred to as a spin move.

In general, any of these apparatuses and methods may be configured to permit active rotation of the rigidizable member. In variations having an inner and outer member, the apparatus and method may permit active rotation of the inner and outer, e.g., telescoping, members. In some examples the apparatus may be configured to perform an active rotation of the outer (mother) member, in which a hook or curve may be formed while pulling proximally. In some examples the apparatus may be configured to include a non-uniform bending stiffness, so that the apparatus may include active rotation of the inner and/or outer members with non-uniform bending stiffness in order to flip and a loop and reduce the curvature of the colon.

Other techniques including active rotation may include active rotation of the outer elongate member (mother), in variations having an outer elongate member as well as an inner elongate member combined with rigidifying or relaxing the outer and/or inner member, forming a hook or curve at the distal end (in the steerable region) and pulling proximally. Any of these methods may also include allowing free rotation as part of the maneuver.

Any of these techniques may include the use of a spin move to maintain the plane of the distal bend or curve as the elongate member otherwise rotates. As mentioned, the apparatus may actuate the steering elements, such as tendons, in order to keep the steerable distal tip region bent or curved in a specific plane relative to the body.

Any of these apparatuses and methods may also or additionally include a distal anchor instead of or in addition to the curve or hook described above, such as an expandable member (e.g., balloon, expandable cage or mesh, etc.) and/or suction. For example, any of these techniques may include anchoring the distal end region, e.g., by expanding a balloon and/or applying distal suction against the lumen wall(s), then pulling proximally to reduce the curvature.

The methods and apparatuses descried herein may be configured to reduce the curvature of the colon by “pleating” the colon. For example, the methods or apparatuses may anchor at a distal end region of the apparatus (e.g., the elongate rigidizable member, or in variations having more than one, e.g., telescoping, elongate members, the inner elongate rigidizable member and/or an outer elongate rigidizable member). Pleating may include folding and/or gathering the colon to shorten the length of the colon and therefore the curvature of the colon. The methods an apparatuses described herein may include pleating the colon by anchoring a distal end of the elongate rigidizable member and pulling proximally; the distal end of the rigidizable member may be anchored by an expandable anchor (e.g., balloon), and/or by applying suction.

In some examples the apparatus or method may include feedback (e.g., force/torque feedback visual feedback, shape sensing feedback, etc.) to assist during the reduction. This may help ensure patient safety. Shape sensing may be used to help perform any of the techniques described herein. Also described herein are methods and apparatuses including vision feedback to help automate reductions. As discussed above, reductions may be fully automated, including using a machine learning agent (e.g., artificial intelligence, AI) or partially automated, including prompting a user (e.g., physician (to consider a recommend reduction technique.

The automated or semi-automated methods and apparatuses including reduction of the colon described herein provide numerous advantages over current methods for endoscopy and related procedures within the colon. For example, some endoscopes currently have the ability to modulate bending stiffness of the scope. Flexibility can be increased to navigate through tortuous anatomies. Once they have navigated through the tortuous anatomies, increasing stiffness can allow the loops to be ‘locked out’. However, if a loop is re-forming, the clinician generally has a limited number of currently possible approaches. For example, the clinician can try further reduction to achieve better one to one effect or reciprocal motion at the distal and proximal ends. The clinician can have someone apply abdominal pressure during reduction. Finally, the clinician may push through the loop. This approach is less advisable but is still commonly practiced. Alternatively and beneficially, systems such as those described herein (e.g., system 2400z/9300z) can be used to provide a fully automated or semi-automated reduction of curvature in a body lumen such as the colon. The system may comprise various features and capabilities that allow for a safe and more reliable reduction in curvature than what is possible using currently available devices and techniques.

In some examples, a proximal end of the inner and outer elongate members (e.g., 2410/9310, 2400/9300) may be able to freely rotate within a floating handle or collar. During a reduction, the system has navigated around a loop or circle. In order to reduce this, the system has to rotate the same number of degrees as the tortuous path it has navigated through.

If the inner member (e.g., 2410, 9310) is hooked at a flexure or tight turn, adding a floating handle or handle to the inner member (e.g., 2410,9310) would enable the system to reduce with no active rotation of the system. As the system is being pulled out, the hook is supported by the anatomy at the distal end. The floating handle then allows the system to untwist at the proximal end instead. In examples where the inner member is sufficiently torsionally stiff, by the end of the reduction, the handle would have rotated nearly 360 degrees.

In the example elongate rigidizable members 2108 shown in FIGS. 21A and 21B the elongate rigidizable members have a proximal end with a rotatable connection that may be configured to freely rotate and/or be driven in a clockwise or counterclockwise direction 2125. For example, the proximal end connector 2122 may be secured into a handle region of the apparatus, and the junction between the proximal end connector and the body of the elongate rigidizable member may allow rotation relative to the two.

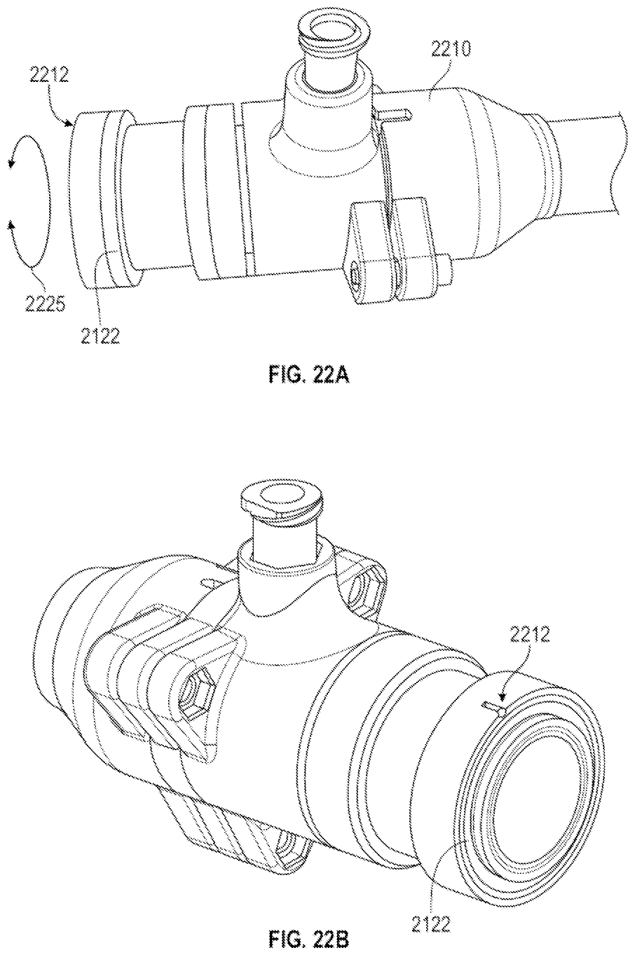

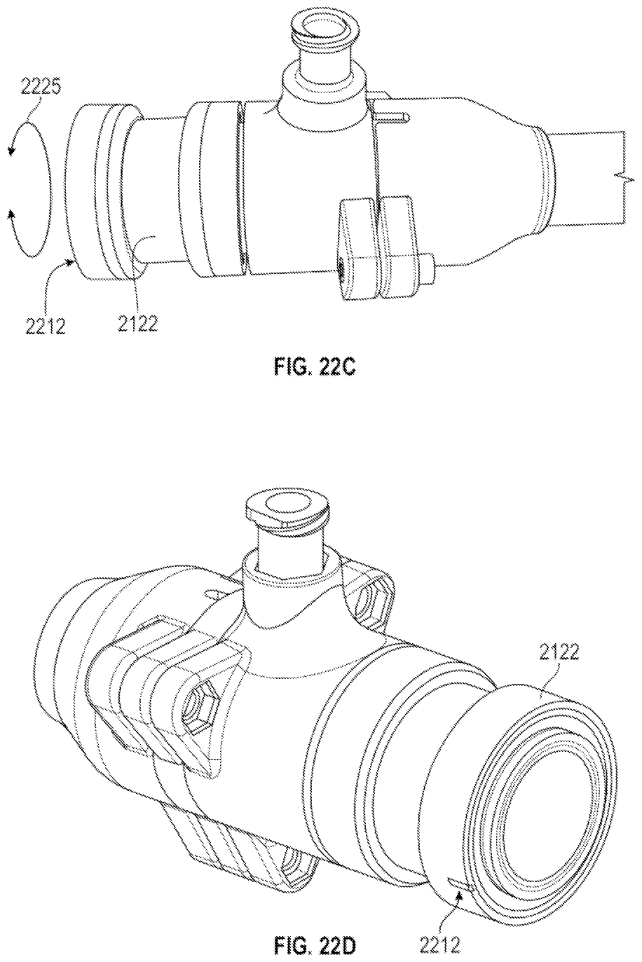

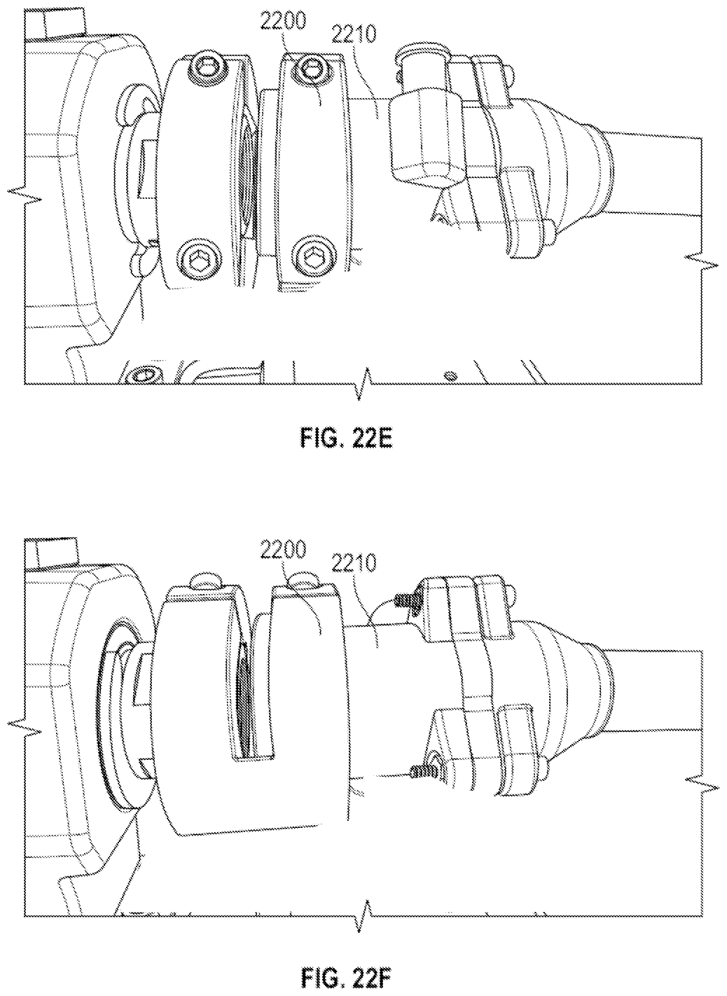

FIGS. 22A-22D illustrate another example of a proximal end of an elongate rigidizable member of an apparatus as described herein. In this example, the apparatus includes a proximal end connector 2122 that is rotatable relative to a more distal handle region 2210 of the elongate rigidizable member. The junction between the proximal end connector 2122 and the distal handle region 2210 may configured to lock (preventing rotation) or be released, allowing rotation. The rotational lock may be automatically operated by the system and/or may be manually operated. Rotation 2225 of the distal handle region 2210 relative to the proximal end connector 2122 may result in rotation of the elongate rigidizable member within the body. In FIGS. 22A and 22B the distal end region of the elongate rigidizable member is shown from the side and from a distal end perspective view. In FIGS. 22A-22B a marker 2212 is shown in a first position. In FIGS. 22C and 22D the distal handle region 2210 has been rotated relative to the proximal end connector 2122, as shown by the change in position of the maker 2212.

In some examples, e.g., including telescoping or nested (parent/child) pairs of elongate rigidizable members may be included in which one or both of the elongate rigidizable members may be configured to rotate as shown in FIGS. 22A-22D. The inner elongate rigidizable member may rotate freely and/or be driven, and/or the outer elongate rigidizable member may be configured to rotate freely and/or be driven. For example, a system may include a handle coupler that may engage with the elongate rigidizable member by one or both of the distal handle region 2210 and the proximal end connector 2122. FIGS. 22E-22F illustrate an example of an elongate rigidizable member in which the distal handle region 2210 of the elongate rigidizable member is held, clamped, in a handle coupler 2200.

The handle coupler may engage with the proximal end connector 2122 to secure it in place and/or in some examples may engage it for driving rotation relative to the distal handle region 2210; in some examples the handle coupler may also engage with the distal handle region 2210. The engagement between either the distal handle region 2210 or the proximal end connector 2122 and/or both may be releasable; releasing just one of the distal handle region 2210 or the proximal end connector 2122 may allow the elongate rigidizable member to freely rotate. As shown by the different rotational positions of the proximal handle region 2210 in FIGS. 22E and 22F, the elongate rigidizable member shown in the example may freely rotate within the handle coupler. A similar but separately controlled configuration may be used (in line) for a telescoping second elongate rigidizable member (e.g., and inner or outer elongate rigidizable member).

In addition to freely rotating, any of the apparatuses described herein may be configured for admittance control rotation, which may provide the experience of freely rotating by driving in the direction of sensed torque. For example, in admittance control rotation, one or more sensors (e.g., a torque sensor) may detect torque on the elongate rigidizable member, as when the user (physician, technician, etc.) rotates the elongate rigidizable member and rotation may be driven based on the sensed torque. Both freely rotating and admittance control rotation handles may be referred to herein as floating handles as they may not have a predetermined driven position or orientation, and/or may rotate unconstrained.

An exemplary method for using a floating (freely rotating) handle follows. In some examples, the inner member (e.g., 2410/9310) may be hooked around a bend in the lumen (e.g., splenic or sigmoid/descending junction, etc.). Alternatively, a balloon can be used to anchor the system to the lumen. Using a balloon may allow the system to anchor to a location other than around a bend.

Once the system is hooked, the inner and outer members (e.g., 2410/9310, 2400/9300) can be de-rigidized. The handle of the inner member can be actuated (e.g., by clutching) to allow the proximal end of the inner member to freely rotate within the handle. In some examples, the proximal end of the inner member is always free to rotate. The system is pulled back until the curve is sufficiently reduced. The inner member will rotate as the curve is reduced.

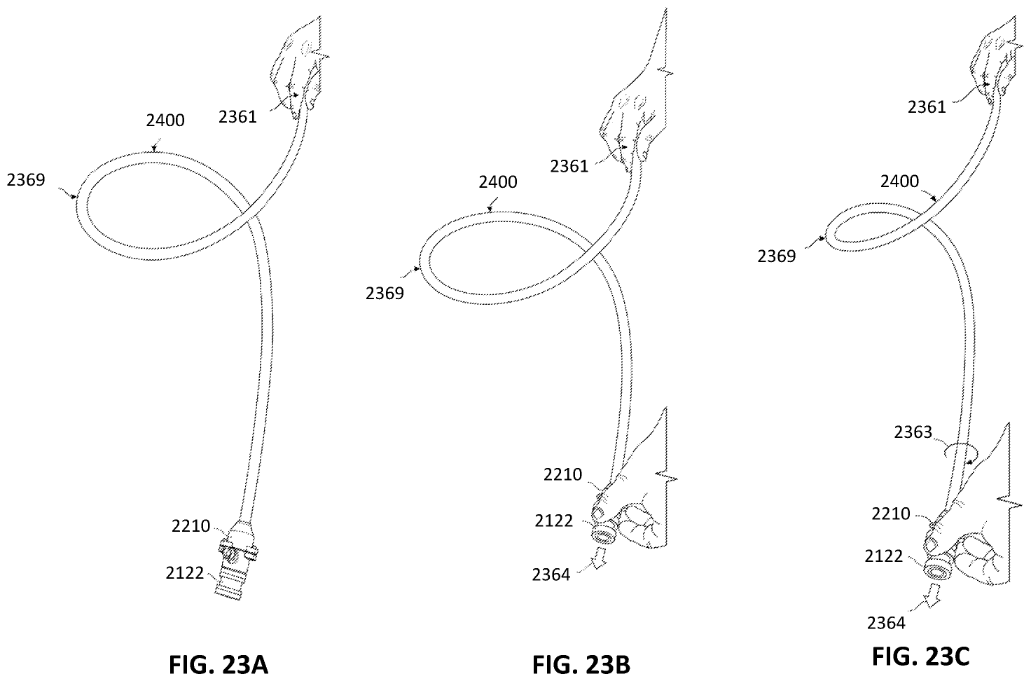

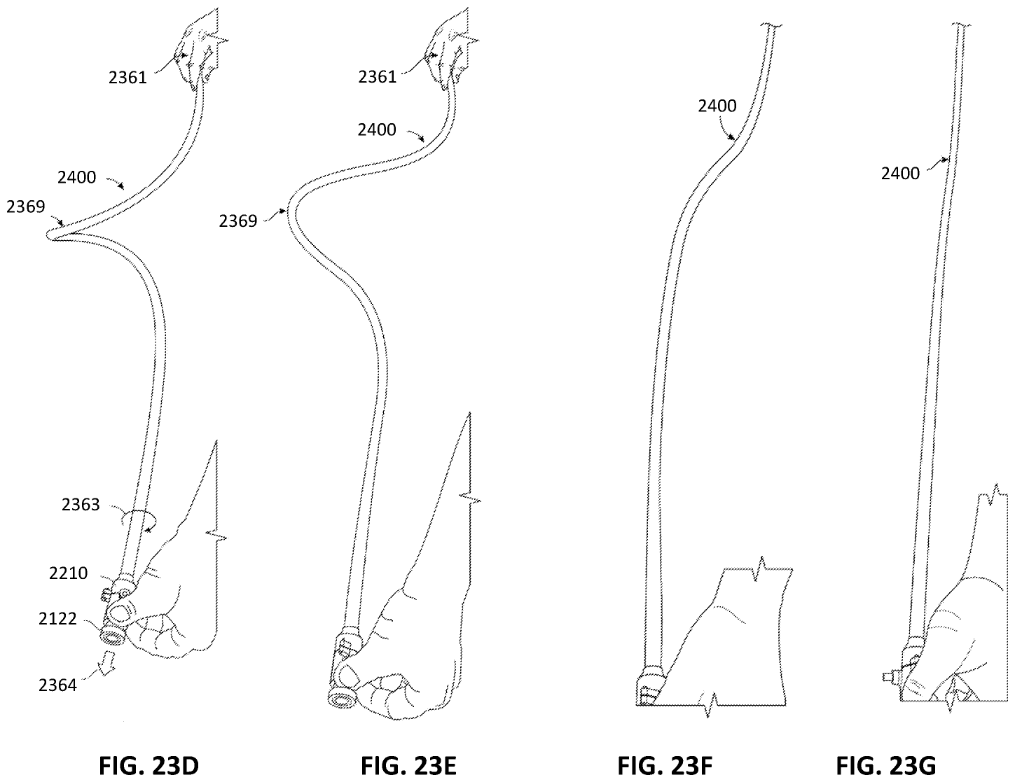

FIGS. 23A-23G illustrate reducing of a loop or curve by allowing an elongate rigidizable member 2400 to rotate at a proximal end, while pulling the proximal end proximally. In some examples the distal end region of the elongate rigidizable member may be anchored or held proximally. For example, the distal end region may be formed into a hook or curve that can be leveraged against the lumen of the colon and/or the distal end region may be anchored by using an expanding or inflating member (e.g., a balloon anchor) or by suction, as described above. FIGS. 23A-23G show the exemplary device outside of the body for convenience, however the same or a similar procedure may be performed within the colon.

In FIG. 23A, the elongate rigidizable member 2400 may be a single elongate rigidizable member or it may be coupled with an inner elongate rigidizable member 2410 to form a system as described herein. The elongate rigidizable member may be initially rigidized to hold the loop or curved shape shown in FIG. 23A. In FIG. 23A the elongate rigidizable member includes a proximal end having a proximal end connector 2122 that is rotatably coupled to a distal handle region 2210 so that it may rotate (e.g., freely, driven, and/or in admittance control rotation). In this example the distal end region is held 2361 in place (e.g., anchored) as mentioned above. The elongate rigidizable member 2410 may then be pulled proximally 2364 while either allowing the distal handle region 2210 to rotate relative to the proximal end connector 2122, either freely or by admittance control rotation, so that the elongate rigidizable member rotates 2363. This is illustrated in FIGS. 23B and 23C. In FIG. 23B the proximal end connector 2122 is held (in this example, manually, but a handle coupler may be used. FIGS. 23D-23G continue this process, pulling the proximal end connector 2122 proximally while the elongate rigidizable member rotates 2363, which results in uncoiling of the loop 2369.

In some examples, the robotic system (e.g., system 2400z/9300z) is configured to maintain lumen-centricity of a distal end of the inner member as it is pulled back and/or rotated. Pulling back on the system can cause a deflected or hooked distal end to contact or dig into the lumen sidewall (e.g., colon wall). Rotating the distal end of the inner member can allow it to remain at or near a center of the body lumen.

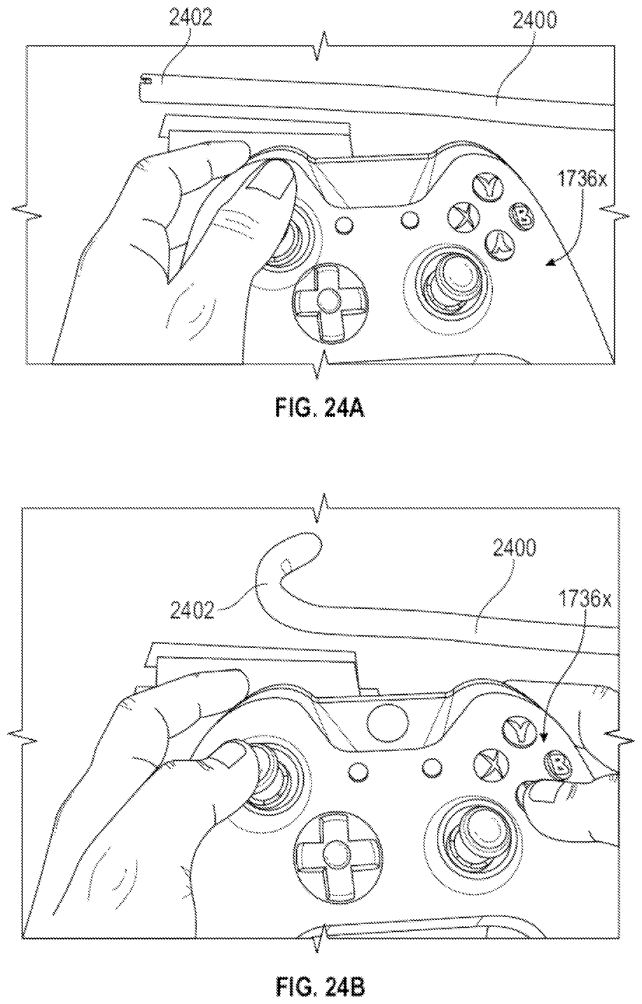

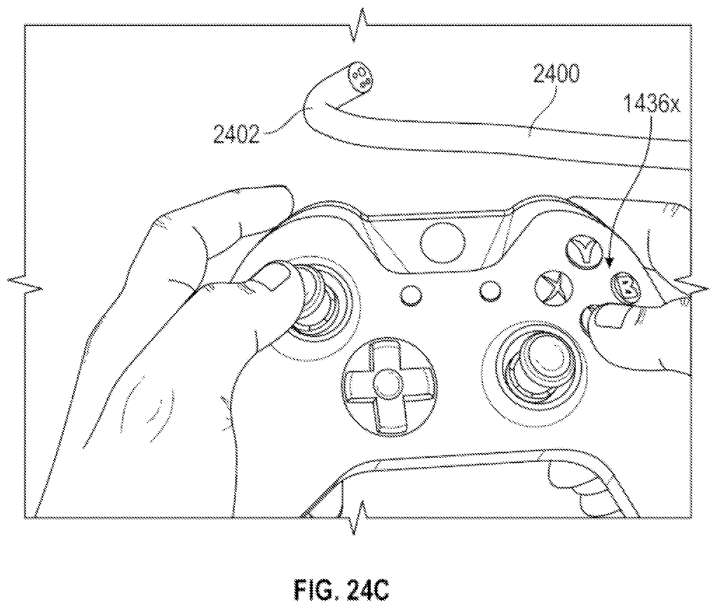

FIGS. 24A-24C show an example of an elongate stiffening member 2400. The distal end 2402 is shown straight or undeflected in FIG. 24A. FIG. 24B shows the distal end 2402 deflected or bent. The bending of the distal end may be controlled by controller 1736x, as described above. Such a maneuver can be used to hook the distal end 2402 into a lumen wall. FIG. 24C shows the elongate member 2400 rotating, as it would do as the system is maintaining lumen-centricity.

An example for maintaining lumen centricity of the distal end of the inner member comprises hooking the distal end of the inner member around a bend of the body lumen (e.g., splenic or sigmoid/descending junction, etc.). A camera or sensor at or near a distal end of the inner member can allow for a view of the lumen. The clinician may de-rigidize the system and pull the system back. As the view of the lumen moves, the clinician may pause and activate a “distal end articulation” macro or program. Execution of this macro actuates a bending section at or near the distal end of the inner member to maintain lumen-centricity. The clinician may continue to pull back the system, causing actuation of the bending section until the loop is sufficiently reduced.

In some examples, the rotation of the distal end can be done automatically by the system based on gathered data. For example, visual feedback from a distal end of the inner member can be analyzed and allow the system to automatically rotate the distal end to maintain a field of view within certain parameters. For another example, shape sensing feedback regarding the shape of the inner member and/or the body lumen can be analyzed and allow the system to automatically rotate the distal end of the inner member to a desired position within the body lumen. Lumen centricity may be helpful to minimize the risk of damage to the colon walls; for example, if visualization of the apparatus (e.g., the distal end of the elongate rigidizable member) is difficult or lost during a procedure, lumen centricity may be maintained to prevent damage to the colon wall.

In some examples, the system does not automatically maintain lumen centricity, but may prompt a clinician to manually rotate the end of the inner member based on gathered data (e.g., visual or shape sensing feedback).

In some examples, the inner member may be partially rigidized while the system is being retracted. This partial rigidization may help prevent sudden unfurling of the loop, allowing the system to better monitor the lumen centricity of the distal end of the inner member.

In some examples, the system (e.g., system 2400z/9300z) is configured to drive rotation of the outer member until a loop or curve in the body lumen is reduced. The rotation allows the loop to come out of plane and increases the “moment arm” to reduce the loop or curve. The clinician may navigate through a loop or curve in the lumen. The clinician can then hook a distal end of the inner member (e.g., 2410/9310) around the curve (e.g., at splenic or sigmoid/descending junction, or hepatic flexure, etc.). In some examples, a balloon can be used to anchor within the lumen, allowing a clinician to anchor at a point away from a curve, if desired. The system may be de-rigidized. The clinician can control the system to actuate rotation of the outer member to a specified rotation (e.g., about 180°).

In some examples, the system utilizes shape sensing to determine whether the system, and therefore, the body lumen, is moving. If the system determines that the system is not moving, rotation continues until it is determined that the system is moving.

In some examples, the system utilizes torque feedback to confirm that rotating the outer member (e.g., 2400/9300) is safe. If the measured torque exceeds an upper limit after a specific amount of rotation (e.g.,) 30° and no shape change was detected, then the system can stop rotation of the outer member.

The clinician can continue to pull the system back until the loop or curve is reduced.

In some examples, any of the described functionality can be performed by a system (e.g., system 2400z/9300z) having a non-uniform bending stiffness in at least one of the inner member (e.g., 2410/9310) and the outer member (e.g., 2400/9300).

In examples in which the outer member has a non-uniform bending stiffness, as the system is advanced through a loop or curve, it can be assumed that the outer member bends along its more flexible axis. When the clinician desires to begin the reduction, they can cause the system to rotate the outer member to apply a torque and flip the loop or curve. As the system is pulled back, the outer member will tend to stay bent along the loop until enough torque is applied to cause the outer member to flip, bending in the opposite direction along the flexible bending axis, causing the loop or curve to reduce.

It will be appreciated that the inner member (e.g., 2410/9310) may also comprise a non-uniform bending stiffness such that the inner member causes the system to flip around upon rotation of the outer member, thereby reducing the curve in the lumen.

In some examples, the non-uniform bending stiffness is formed by insertion of an element having a rectangular cross-section within the elongate member wall. Other configurations are also possible. For example, wires or fibers in the elongate member walls can be wound or arranged to create a preferred and non-preferred bending axis.

An exemplary method of utilizing an outer member (e.g., 2400/9300) having a non-uniform bending stiffness to reduce a curve or loop comprises navigating the system through the loop or curve. The clinician can hook or anchor a distal end of the inner member at a loop or curve (e.g., splenic or sigmoid/descending junction, etc.,). In some examples, a balloon can be used to anchor the system to the body lumen, advantageously allowing for the anchoring site to be away from a curve or loop. After anchoring, a clinician may de-rigidize the system. The clinician may cause the system to rotate a proximal end (e.g., handle) of the outer member to a specified amount of rotation corresponding to the more flexible bending axis of the outer member.

In some examples, the system utilizes shape sensing to determine whether the system, and therefore, the body lumen, is moving. If the system determines that the system is not moving, rotation continues until it is determined that the system is moving.

In some examples, the system utilizes torque feedback to confirm that rotating the outer member is safe. If the measured torque exceeds an upper limit after a specific amount of rotation (e.g., 30°, or 20-40°, etc.) and no shape change was detected, then the system can stop rotation of the outer member.

The clinician can continue to pull the system back until the loop or curve is reduced.

It will be appreciated that various features and functionalities described herein can be combined. For example, in some examples, (e.g., system 2400z/9300z) is configured to drive rotation of the outer member (e.g., 2400/9300) until a loop or curve in the body lumen is reduced and also comprises a “floating” handle, allowing a proximal end of the inner member to freely rotate within a handle or collar at its proximal end. The addition of the floating handle may advantageously enhance the reduction because the floating handle would allow the child to twist, removing any twisting that may otherwise occur at the proximal end, while maintaining the relative position of the distal end anchor steady during the reduction.

In such examples, the clinician may navigate through a loop or curve in the lumen. The clinician can then hook a distal end of the inner member around the curve (e.g., at splenic or sigmoid/descending junction, etc.). In some examples, a balloon can be used to anchor within the lumen, allowing a clinician to anchor at a point away from a curve, if desired. The system may be de-rigidized. The clinician can control the system to actuate rotation of the outer member to a specified rotation (e.g., about 180°).

In some examples, the system utilizes shape sensing to determine whether the system, and therefore, the body lumen, is moving. If the system determines that the system is not moving, rotation continues until it is determined that the system is moving.

In some examples, the system utilizes torque feedback to confirm that rotating the outer member is safe. If the measured torque exceeds an upper limit after a specific amount of rotation (e.g., 30°) and no shape change was detected, then the system can stop rotation of the outer member.

The clinician may actuate the system to allow the inner member proximal end to freely rotate (e.g., by clutching a handle at its proximal end). The clinician can then continue to pull the system back until the loop or curve is reduced as the inner member rotates at its proximal end to untwist.

As described herein, in some examples, the system (e.g., system 2400z/9300z) may utilize a balloon to anchor either the inner or outer member to the body lumen. Utilizing a balloon instead of hooking a distal end of the inner member to a curve in the lumen may advantageously allow for anchoring at any location within the body lumen, including locations positioned away from curves or loops.

In some examples, the system (e.g., system 2400z/9300z) may utilize suction (e.g., using an air chuck) to anchor either the inner or outer member to the body lumen. Utilizing suction instead of hooking a distal end of the inner member to a curve in the lumen may advantageously allow for anchoring at any location within the body lumen, including locations positioned away from curves or loops. Initiating suction may also advantageously be performed more quickly than, for example, inflation of a balloon.

In some examples, the system (e.g., system 2400z/9300z) is configured to reduce curvature or a loop in a lumen by retracting portions of the lumen, thereby causing a portion of the lumen proximal to the retracted portion to reduce in length by gathering, folding together, or pleating.

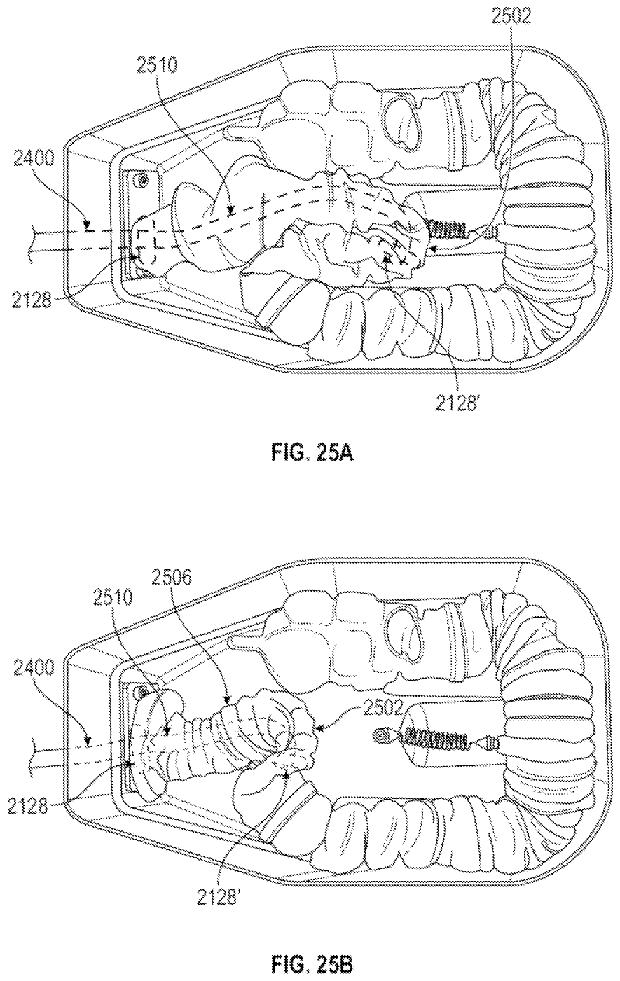

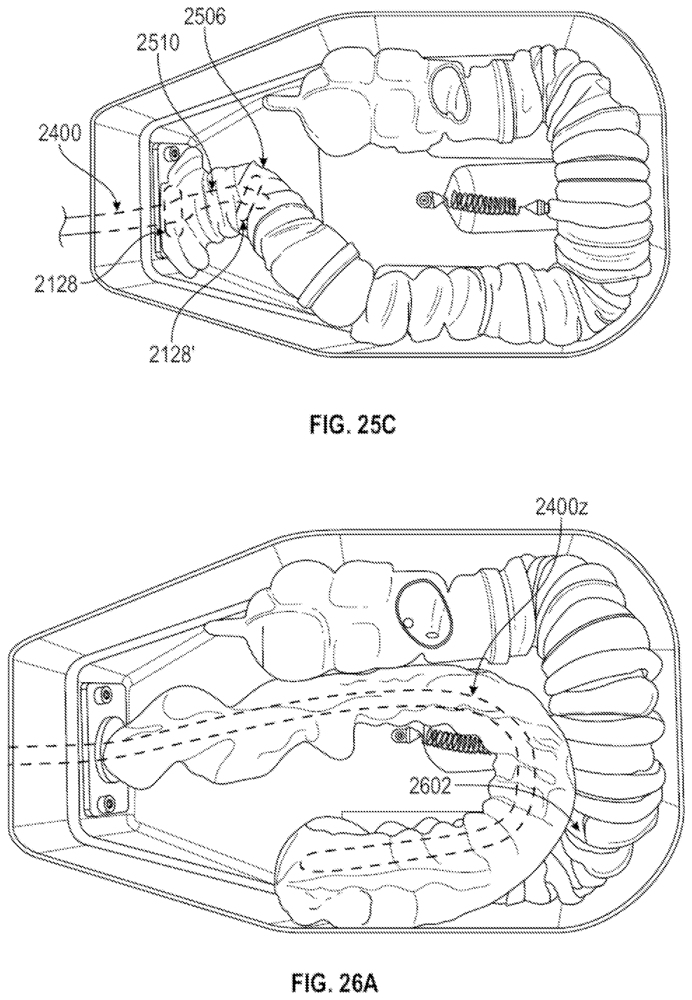

FIGS. 25A-25C show an example of such a method being performed in a colon. In this examples, a clinician may control the system (e.g., system 2400z/9300z), including an outer elongate rigidizable member and an inner elongate rigidizable member, to navigate a curve or bend 2502, as shown in FIG. 25A. The outer elongate rigidizable member (e.g., 2400/9300) may be held proximal of the telescoping inner member (e.g., 2410/9310). An anchor (e.g., a balloon 2128′ or other expandable member) on the inner elongate rigidizable member may be actuated (e.g., expanded) to anchor the system to the lumen. In FIG. 25A, the distal anchor is an annular balloon 2128′ that has expanded within the colon. The system may remove or reduce the rigidity of the inner (and/or outer) elongate rigidizable member and may withdraw the anchored inner elongate rigidizable member proximally to reduce the curvature. In some examples, the inner elongate rigidizable member may be pulled proximally until either it cannot be pulled back more or until visual feedback from a distal end of the inner member is withdrawn to the outer member position. In some examples both the inner and outer elongate rigidizable members may be withdrawn proximally. Either a single distal anchor may be used or a pair of anchors, a first (distal) anchor 2128′ on the distal end region of the inner elongate rigidizable member and a second (proximal) anchor 2128 on the outer elongate rigidizable member may be used. FIGS. 25A-25C show both a distal anchor 2128′ and a proximal anchor 2128. The distal anchor 2128′ may be drawn proximally towards the proximal anchor 2128 to reduce the colon. The resulting pleating of the colon shown in FIG. 25C provides a less tortious, shorter path, and may simplify insertion of one or more therapeutic tools through the system, as well improve visualization of the colon. In FIG. 25B the colon has been partially reduced by pulling back on the inner elongate rigidizable member 2400z, reducing curve 2502 and forming pleats or gathers 2506.

Any of the systems described herein may include one or more sensors to provide force feedback to ensure that the retraction of the lumen is performed under reasonable force. If the measured force spikes above a predetermined threshold (e.g., due to adhesions or scarring), the retraction can be terminated. Alternatively or additionally, the partially rigidizing examples (described in greater detail below) may prevent damage to the colon by reducing the force between the system 2400z and the walls of the colon.

Once the colon has been reduced sufficiently, which may be determined by the user manually using imaging into or through the lumen and/or by shape sensing of the system, or may be performed automatically, e.g., in which the system indicates a clear or sufficiently straightened lumen and/or a limit based on the forces applied to the colon, the system may maintain the reduced state of the colon by rigidizing the inner and/or outer elongate rigidizable members, allowing further advancement. For example, the inner elongate rigidizable member may be advanced while the outer elongate rigidizable member remains rigidized to hold the colon in a reduced configuration until the inner elongate rigidizable member reaches another curve or bend. This process can be repeated.

In some examples, instead of using a balloon to anchor to the lumen, suction may be used. Examples of suction ports are shown and described in FIGS. 21B-21F, above. The suction may be applied through a working channel of the inner member or a suction channel. The suction channel may be helically wound around the elongate rigidizable member. In some examples, the suction is applied through the annular space between the inner and outer members. The suction can be applied to anchor the inner elongate member to the lumen of the colon in the same way that the balloon anchor(s) shown in FIGS. 25A-25C were described. In some examples just the inner elongate rigidizable member includes a suction port/anchor. In some examples both the inner elongate rigidizable member and the outer elongate rigidizable member may include a suction port that may anchor to the wall(s) of the colon. The inner elongate rigidizable member may be anchored to the entire circumferential region of the colon, or to a portion of the circumference of the colon lumen.

In any of the reduction techniques described herein the rigidity of the elongate rigidizable member may be set to an intermediate rigidity, e.g., neither fully flexible nor fully rigid, to enhance the ability of the apparatus to safely and rapidly reduce the tortuosity of the colon. This may be referred to herein as partial rigidization. For example, the system (e.g., system 2400z/9300z) may be configured to vary the stiffness of the inner elongate rigidizable member and the outer elongate rigidizable member such that they can transition from a fully flexible configuration (that is freely and easily bendable) to a more rigid shape (which may be arbitrarily formed by the user steering the apparatus) that is not fully rigid but may be deformable within a predetermined, and in some examples selectable force. This allows the apparatus to deform from the partially rigid shape (e.g., to “give”) at a force that is less than the force that may cause damage to the colon. The user may select this relative stiffness (increasing/decreasing it) and/or the system may adjust the relative stiffness, dynamically during performance of a reduction maneuver, which may further prevent harm to the patient.

A system and method for reducing the curvature of a colon using partial (or variable) rigidization may be performed using shape sensing, force sensing and/or torque sensing. For example, the system may use shape sensing of the apparatus to provide an image of the shape of the inner and/or outer elongate rigidizable members while withdrawing them proximally in a rigid or partially rigid configuration. If the force acting on the inner and/or outer elongate rigidizable member resisting proximal withdrawal increase to a threshold which may be preset of user defined (e.g., 5 N, 7N, 10N, 12N, 15N, etc.), and the shape of the inner and/or outer elongate rigidizable member remains curved (e.g., is not unfurling), the system may automatically or manually adjust the rigidity, e.g., to decrease the rigidity slightly by an increment (e.g., 5%, 10%, etc.) until the force on the inner and/or outer elongate rigidizable member is decreases and the shape, as detected by the shape sensor, changes. If the force reduces too much, and/or the shape bends more than desired the rigidity may be increased again, e.g., to a rigidity that is less than the original rigidity but more than the current rigidity.

The increment by which the rigidity is increased or decreased may be a function of the positive or negative pressure applied. For example, the apparatus may include an actual or estimated maximum rigidity based on the maximum pressure (positive or negative) applied by the apparatus to rigidize the inner and/or outer elongate rigidizable member. The rigidity of the inner and/or outer elongate rigidizable member may be set to between the minimum (e.g., no applied pressure) and this maximum pressure and therefore rigidity. The apparatus may alternatively allow selection of any intermediate pressure, and therefore rigidity of the inner and/or outer elongate rigidizable member. For example, the apparatus may allow selection of a percentage of the maximum pressure (positive or negative pressure) or in some cases as a percentage of the maximum rigidity, e.g., 10%, 15%, 20%, 25%, 30%, 35%, 40%, 50%, etc. of the maximum pressure or rigidity. The apparatus may include a control (e.g., button, dial, knob, etc. which may be virtual or actual) to allow the user to manually adjust the rigidity/pressure applied.

In some examples the percentage of the rigidity may be mapped to the applied pressure (positive or negative pressure), since the relationship between the applied pressure and the rigidity may be non-linear. This mapping of pressure applied to rigidity of the apparatus, e.g., of the inner and/or outer elongate rigidizable member, may be pre-calibrated by the apparatus. Thus, the apparatus may use this mapping to adjust the applied pressure to deliver a manually or automatically (or semi-automatically) selected rigidity during operation, including during a reduction procedure including intermediate rigidization.

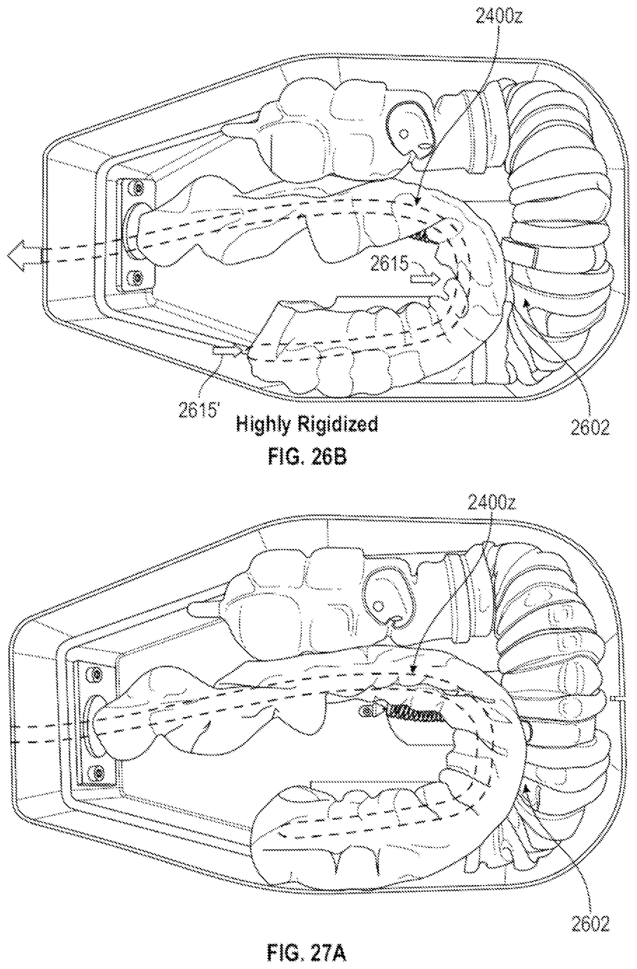

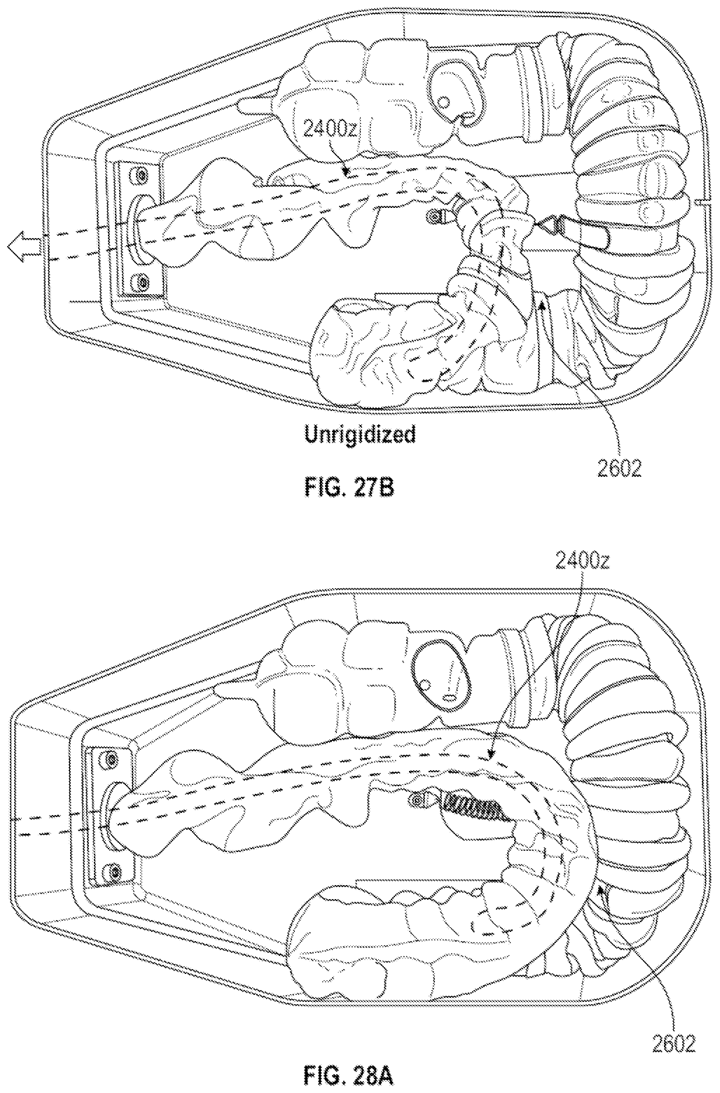

FIGS. 26A and 26B show and example of a system in which the inner and outer elongate rigidizable member is fully rigidized. In this example, the shape may be set and highly (or fully) rigidized after navigating around the bend 2602 in the colon, as shown in FIG. 26A. The apparatus may show the shape of the system 2400z (inner and/or outer elongate rigidizable member(s)), rigidized in the curved configuration. If the rigidized system is pulled proximally, as shown in FIG. 26B, the inner and/or outer elongate rigidizable member(s) may be driven against the lumen of the colon with a force at one or more regions 2615, 2615′ that may harm the colon. The rigidized system 2400z in this example may bend slightly in response to the force pulling it against the lumen wall but this force may be substantial and may result in damage to the colon. In contrast, if the system is withdrawn proximally when fully un-rigidized, as shown in FIGS. 27A-27B, the inner and/or outer elongate rigidizable member(s) may not be able to apply sufficient force to reduce the curvature. FIG. 27A is identical to FIG. 26A, however FIG. 27B the system 2400z is not rigidized. The apparatus may display the force acting between the system 2400z and the lumen of the colon, which may be relatively small, and the shape (based on a shape sensor) of the system 2400z.

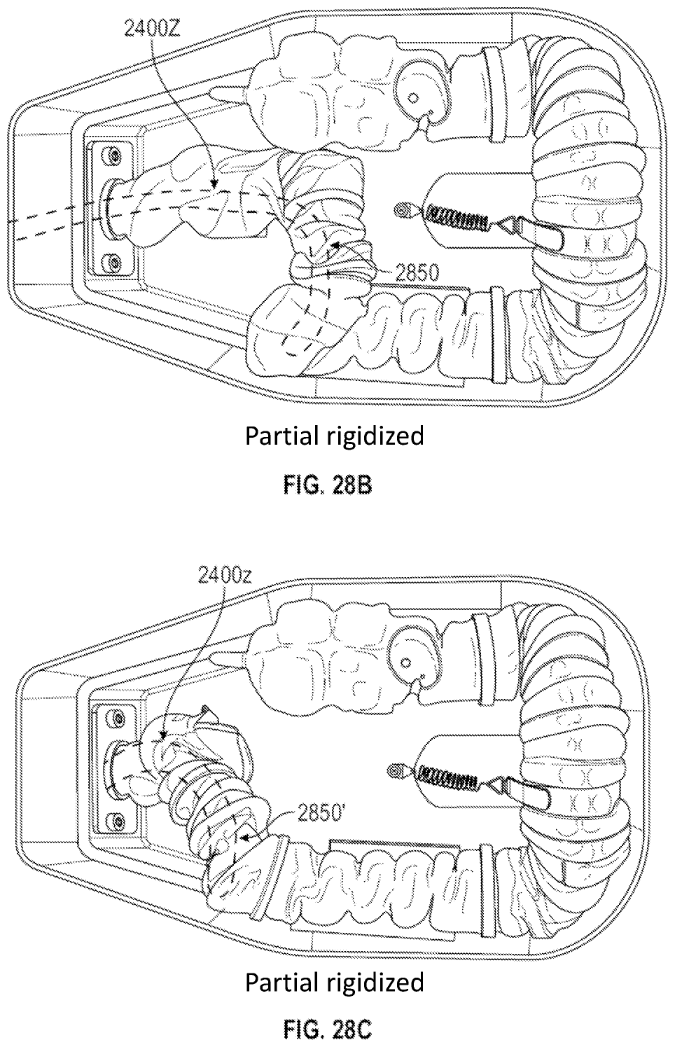

FIGS. 28A-28C illustrate the use of a partially (or incrementally) rigidizing apparatus in reducing the curvature of the colon. FIG. 28A shows the same starting configuration as FIGS. 26A and 27A, with the system 2400z (e.g., having an inner and outer elongate rigidizable member) within the lumen of the colon forming a curve that is initially highly rigid. The apparatus may display to the user the force(s) acting on the elongate rigidizable member(s), including in some examples the torque acting on the elongate rigidizable member. The apparatus may also display an image of the shape of the elongate rigidizable member(s). During a procedure, the user may reduce the curvature of the colon by withdrawing the system 2400z (e.g., the inner and/or outer elongate rigidizable members) proximally, while concurrently displaying the force on the system and/or the shape of the elongate rigidizable member(s). In addition to the force measurements, and in some cases instead of an explicit force measurement, the user may feel the resistance to withdrawing the system proximally. If the sensed force(s) exceed a threshold the apparatus may reduce the pressure rigidizing the elongate rigidizable member(s), as shown in FIG. 28B (showing a partially rigidized system 2400z), thereby reducing the rigidity of the system. The rigidity and/or pressure may be reduced by a percentage (e.g., by approximately 5%, 10%, 15%, 20%, 25%, 30%, etc., or any increment therebefore), including dynamically reducing the pressure and rigidity while pulling the system proximally and, in some cases, observing the shape of the system. Thus, the apparatus (e.g., the inner elongate rigidizable member) may be rigidized such that it can just barely overcome the natural bending stiffness of the outer member but can still be deformed before the force applied by the elongate rigidizable member exceeds a threshold that may otherwise damage the colon. In FIG. 28B, the rigidity/pressure may be decreased to allow the system 2400z to bend (shown by arrow 2850) and “give” slightly, while retaining enough resistance to bending so that the system continues to pull the colon proximally while pulling the system proximally. In some examples the user may feel the force exerted by the system in response to pulling proximally (e.g., the resistance to pulling), and if the feel of the force feels too high, may trigger a control (e.g., button, dial, slider, etc.) to release additional pressure rigidizing the system. Alternatively or additionally, the system may automatically or semi-automatically detect the force (from one or more force sensors) and may either alert the user to reduce the pressure and therefore decrease the rigidity or may automatically reduce the pressure rigidizing the system. In some instances, if either or both the force sensed feels too low (e.g., falls below and minimum force threshold), the apparatus may increase the pressure and therefore the rigidity of the system. In FIG. 28C, after reducing the rigidity further the system may be withdrawn to reduce the colon as shown. Further reduction may be achieved by rotating or otherwise manipulating the system. Optionally, the system restore rigidity, e.g., of the outer elongate rigidizable member, to maintain the reduced shape so that the inner elongate member may be advanced and/or the system may be advanced within the reduced colon to allow one or more procedures to be performed.

In one example of a reduction using partial rigidization, the user may navigate the system to a curve or loop in the body lumen. A distal end of the system may be hooked against a portion of the lumen, and the system may be withdrawn proximally. The clinician can de-rigidize either the outer or inner elongate rigidizable member while the other member (e.g., the inner elongate rigidizable member, is partially rigidized by the application of pressure (e.g., positive or negative pressure). The user can pull proximally to retract the system while adjusting the rigidity of the other member based on tactile feedback (e.g., the resistance to pulling proximally and/or to rotation), visual feedback (e.g., looking at a shape of the system that is detected, e.g., by a shape sensor), and/or a measure of the actual force being applied against the system as it is withdrawn proximally and/or rotated, and/or based on a view from one or more cameras on the device, e.g., at a distal end of the system, which may confirm that the lumen is in clear view.

As mentioned, shape sensing feedback can be used to confirm that the loop or curve is reduced while performing this reduction technique. Optical (fiber optic) shape setting may be used. In some examples, feedback regarding force can be used to confirm that forces are within a predetermined range at or around the distal tip system handle during withdrawal. Sensed feedback may be displayed on a display or screen and may be presented as an absolute or normalized numeric and/or graphical number. In some examples, the robotic system (e.g., 2400z/9300z) is configured to receive feedback regarding forces applied at various components and at various locations within the system. Gathering information about the forces applied can allow the robotic system to ensure that the movement of the system is safe and reliable.

In some examples, the robotic system is configured to sense rotational torque applied to handles or proximal ends of the inner and/or outer members. The robotic system can use gathered force data to assist a clinician during a reduction and inform them if any maneuver they are attempting exceeds a predetermined range of reasonable forces/torques.

For example, in some examples, during a reduction of a loop (e.g., an alpha loop), rotation may be applied to the outer member (e.g., 2400/9300). If the torque applied to the outer member handle or proximal end does not allow the system to rotate the necessary amount, the system can deduce that there are adhesions on the colon or scar tissue preventing lumen movement, limiting the system's ability to manipulate the lumen walls. In certain such examples, the robotic system may automatically stop the reduction technique.

As described above, in some examples, the robotic system (e.g., 2400z/9300z) is configured to receive feedback regarding the shape of the system. This feedback can comprise sensing the shape of the inner member (e.g., 2410/9310), the outer member (e.g., 2400/9300), or both the inner and outer members. The shape sensing can comprise fiber optic shape sensing. Other examples are also possible (e.g., EM shape sensing).

In some examples, shape sensing can be performed based on a record of the system's movements throughout the body lumen. The robotic system can be configured to analyze the historical record of the movements and, based on those, extrapolate a current shape for the system.

Shape sensing capability can advantageously allow for accurate interpretation of the system as it is navigated through a body lumen. The shape of the system can be assumed to be the shape of the body lumen through which the system has navigated. The system being able to determine the shape of the body lumen, in combination with reduction techniques described herein, can allow the robotic system or partially or fully automate the reduction process.

An exemplary method of using shape sensing to facilitate an automated reduction process follows. In some examples, while navigating the system through the colon, an alpha loop forms. Once the clinician gets to a suitable or stable position within the colon (e.g., near the splenic flexure) they can de-rigidize the inner and outer members. The system can use shape sensing feedback to automatically determine how much or how long to perform a reduction technique (e.g., rotation of the outer member, articulation of the inner member distal end, etc.) until sufficient reduction is achieved.

If the reduction technique fails, and the system begins to slip back, the system can automatically rigidize in place to prevent further losing its position.

By sensing the shape, the robotic system is capable of assisting the clinician during reduction and informing them how close they are to sufficient reduction. The robotic system can also be configured to recommend a specific reduction technique or techniques to the clinician.

In some examples, the robotic system is configured to sense the shape of the loop (e.g., N loop, Alpha loop, reverse alpha loop, gamma loop, transverse loop, etc.) and chooses reduction techniques necessary to reduce loop.

As described above, in some examples, the robotic system (e.g., 2400z/9300z) is configured to receive visual feedback from the system.

The robotic system can receive visual feedback from a distal end of the inner and/or outer member (e.g., 2410/9310, 2400/9300). In some examples, the robotic system can automatically maintain a lumen-centric view, no matter what is going on behind the tip. For example, during an alpha loop reduction, an articulated distal end of the inner member anchored to the lumen wall would actuate automatically to maintain the lumen view as the system pulls back.

In some examples, the visual feedback can be used by the robotic system to provide assistance to the clinician during a reduction technique. The robotic system can use the visual feedback to inform the clinician what direction(s) they should steer to maintain a lumen-centric view.

In some examples, the robotic system can utilize the visual feedback to actuate the steering system in order to maintain lumen-centricity. For example, in some examples, the robotic system may employ artificial intelligence algorithms to actuate the steering cables in order to maintain lumen-centricity.

As described above, in some examples, using artificial intelligence and/or machine learning, the robotic system (e.g., 2400z/9300z) autonomously performs a reduction based on information gathered from earlier reductions.

The robotic system can use any combination of feedback gathered from the system's sensors (e.g., force/torque feedback, shape sensing feedback, visual feedback, etc.).

For example, in some examples, the clinician may navigate through a curve or loop. The robotic system can sense the shape of the lumen (e.g., the colon) throughout the intubation. In the middle of the intubation, the robot determines that a loop has been formed (e.g., N-loop, alpha loop, reverse alpha loop, gamma loop, transverse loop, etc.). For example, the apparatus may determine that a loop has formed by one or more sensors (shape sensors, visual sensors, etc.). The robotic system can then recommend the clinician perform a reduction based on artificial intelligence and/or machine learning. In some examples, the robotic system can also recommend how to perform the reduction, based on artificial intelligence and/or machine learning algorithms.

The clinician can either accepts recommendation or continue forward until forces are too high to continue. If the clinician decides to accept the recommendation, they can de-rigidize the system. The clinician can then perform the reduction based on the recommendation based on a combination of feedback and data gathered from numerous previous reductions. In some examples, the robotic system can use force/torque feedback to consistently check rotational and withdrawal forces during reduction are reasonable with respect to the shape of the loop and/or lumen.

In some examples, instead of the robotic system recommending whether or how to perform a reduction, the robotic system can automatically perform one or more reduction techniques or maneuvers. The robotic system can select the reduction technique(s) using artificial intelligence and/or machine learning algorithms based on stored information from numerous previously performed reductions.

In some examples, once the reduction is completed, the robotic system returns control to the clinician. The clinician can then verify 1:1 reciprocal movement and continue to intubate.

It will be appreciated that the terms ‘reduction’ and ‘reducing’ with respect to features including, but not limited to, a curve, curvature, bend, loop, etc. in a body lumen does not always mean that the feature is completely eliminated. Instead, these terms can mean that the feature is moved towards a straighter or less curved or bent configuration.

In some examples the apparatuses and methods described herein may be used to reduce a loop (e.g., N loop, Alpha loop, reverse alpha loop, gamma loop, transverse loop, etc.) during a procedure using a nested pair of rigidizing members without hooking the distal end of either (or both) the inner and outer rigidizing members. This may be performed by active rotation of either both the inner (e.g., child) rigidizing member and the outer (e.g., mother) rigidizing member by the robotic system, or by active rotation of just the inner rigidizing member.

FIG. 31 illustrates a first example of a method for reducing a loop of a nested pair of rigidizing members within a body lumen, which may also be referred to as reducing the curvature of a body lumen (e.g., colon). This method may be performed by a system, e.g., a robotic system, as described herein. The method illustrated in FIG. 31 may be particularly useful to reduce an alpha or reverse alpha loop, and does not request attachment, e.g., by hooking, of the lumen by the distal end region of the nested apparatus. In practice the system may apply torque to the nested apparatus, e.g., in the direction of least resistance to torque, and may thereafter pull proximally; the torquing allows the loop to move out of plane it was originally in and may increase the moment arm to reduce loop. In FIG. 31, rotation is applied to both the outer and the inner elongate members of the nested apparatus.

For example, the nested apparatus may be advanced through the colon to at least the descending colon 3101 by alternately rigidizing and making flexible the inner rigidizing member and the outer rigidizing member, and steering either the inner or outer (or both) rigidizing members, as described above. A loop of the nested apparatus may be detected 3103. Any appropriate detection mechanism may be used. For example, in some examples the apparatus may detect or determine the presence of a loop (e.g., alpha loop or reverse loop) by one or more of a shape sensor, visual sensor, mapping system, or the like. In order to reduce the loop both the inner rigidizing member and the outer rigidizing member may be de-rigidized (e.g., transitioned to a relaxed configuration) 3105. Both the inner rigidizing member and the outer rigidizing member may then be rotated (by rotation of the proximal handle regions of each) to a rotational angle of between about 160-540 degrees 3107. In some examples shape sensing of either (or both) the inner rigidizing member and the outer rigidizing member may be monitored to confirm that the colon is moving; if movement is not confirmed then the inner rigidizing member and the outer rigidizing member may be further rotated in the same direction until the loop starts to move. In any of these examples, torque sensing may be used to provide feedback to assure that rotation of the inner rigidizing member and/or the outer rigidizing member remains within safe parameters. If the sensed torque, or if the rate of change of the sensed torque, exceeds a safety threshold then the rotation may be stopped 3109. The safety threshold may be user defined or may be set (e.g., about 1 N*m, about 2 N*m, about 3 N*m, about 4 N*m, about 5 N*m, about 6 N*m, about 7 N*m, about 8 N*m, about 9 N*m, about 10 N*m, etc.). Once the loop moves out of the plane it was originally in (e.g., once the inner rigidizing member and the outer rigidizing member have been rotated together to between 160-540 degrees), the nested apparatus may be pulled back from the proximal end 3111. During withdrawal of the proximal end of the nested apparatus, the steerable distal end region of either the inner rigidizing member or the outer rigidizing member may be continuously steered to maintain lumen-centricity of the distal end region. Optionally, if the distal end region begins to move proximally (e.g., starts withdrawing), then the inner rigidizing member and the outer rigidizing member may be further rotated, e.g., another 40-60 degrees and the withdrawal of the proximal end may be continued until the loop is sufficiently reduced 3113.

In some examples the method described in FIG. 31 may be modified as shown in FIG. 32, so that just the inner rigidizing member is actively rotated, and the outer rigidizing member is not actively rotated, but is allowed to float. This example has one less degree of freedom as compared to the technique shown in FIG. 31 in which both the inner rigidizing member and the outer rigidizing member were rotated. As described above, this technique may be performed without hooking (or otherwise attaching) the distal end region of the nested apparatus, e.g., by forming a hook in the steerable distal end region of the inner rigidizing member or the outer rigidizing member. Instead, during an alpha or reverse alpha loop reduction, once the distal end region of the nested apparatus has been navigated (as described above) to or beyond the descending colon, the inner rigidizing member maybe torqued slightly clockwise (or in the direction of least resistance) and then pulled. As described above, torquing causes the loop to come out of plane it was initially in, and may increase the moment arm to reduce the loop.

Removing the need for holding the distal end of the nested apparatus while still allowing for consistent and successful reduction of loops may permit the user or the system to maintain lumen-centricity by steering the distal end region of the inner rigidizing member during the reduction process. In any of these methods, the handle of the outer rigidizing member may be allowed or set to rotate freely (e.g., “float”) at the base/handle, or in some examples the outer rigidizing member may be sufficiently rotationally compliant so that it may be rotated by the inner rigidizing member.

As shown in FIG. 32, the nested apparatus (e.g., the nested inner rigidizing member and the outer rigidizing member) may be navigated through a loop and the distal end region of the nested apparatus may extend at least to the descending color or beyond 3201. A loop of the nested apparatus may be detected 3203. To reduce the loop, both the inner rigidizing member and the outer rigidizing member may first be de-rigidized (e.g., transitioned to a relaxed configuration) 3205. Then, just the inner rigidizing member (and not the outer rigidizing member) may then be rotated, e.g., by rotation of the proximal handle regions of, to a rotational angle of between about 160-540 degrees 3207. In some examples, the shape of the inner rigidizing member and the outer rigidizing member (or both) may be monitored to confirm that the colon is moving out of the plane; if movement is not confirmed then the inner rigidizing member and the outer rigidizing member may be further rotated in the same direction until the loop starts to move 3209. In any of these examples, torque sensing may be used to provide feedback to assure that rotation of the inner rigidizing member and/or the outer rigidizing member remains within safe parameters. If the sensed torque, or if the rate of change of the sensed torque, exceeds a safety threshold then the rotation may be stopped. Once the loop moves out of the plane it was originally in (e.g., once the inner rigidizing member has been rotated together to between 160-540 degrees), the nested apparatus may be pulled back from the proximal end 3211. During withdrawal of the proximal end of the nested apparatus, the steerable distal end region of either the inner rigidizing member or the outer rigidizing member may be continuously steered to maintain lumen-centricity of the distal end region. Optionally, if the distal end region begins to move proximally (e.g., starts withdrawing), then the inner rigidizing member and the outer rigidizing member may be further rotated, e.g., another 40-60 degrees and the withdrawal of the proximal end may be continued until the loop is sufficiently reduced 3213.

The methods described herein may be modified so that the loop is at least partially reduced without rotating the inner rigidizing member, or the inner and outer rigidizing members (e.g., as shown in FIGS. 31 and 32). For example, once the user is in a position to reduce the loop (e.g., an alpha loop or reverse alpha loop), the inner and outer rigidizing members may be made flexible, and both the inner and outer rigidizing members may be pulled back (posteriorly) without rolling. This may reduce the diameter of the loop first. Once the distal end of the inner and outer rigidizing members starts to move proximally (e.g., starts to slip back), the inner or inner and outer elongate rigidizing members may be rotated; for example, between about 160-540 degrees, and then the inner and outer rigidizing members may be pulled proximally again, as described above.

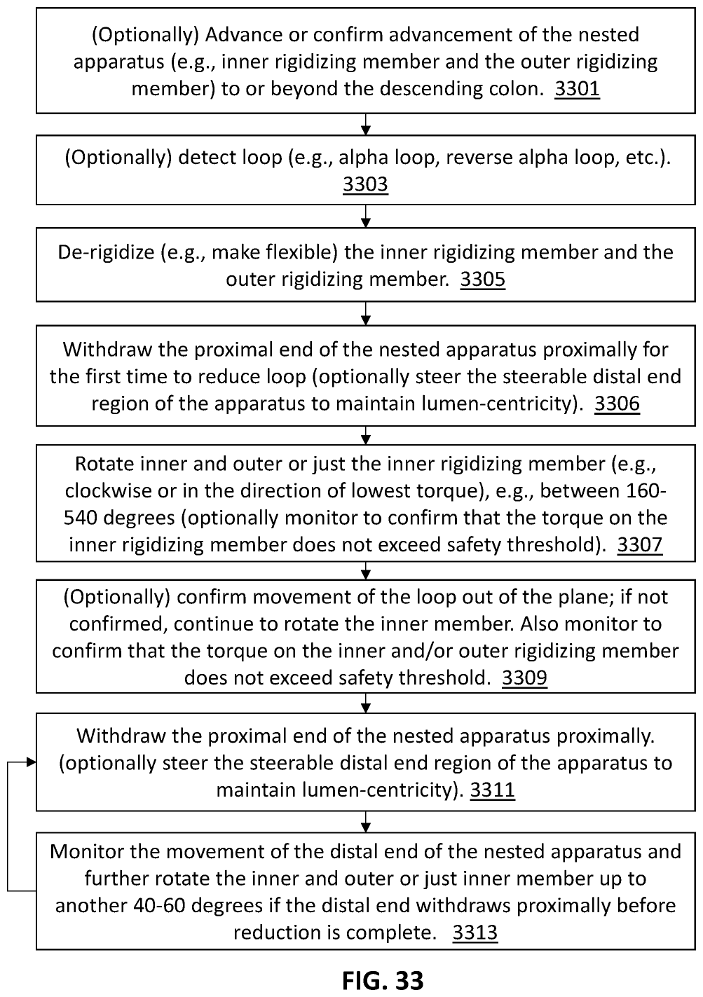

For example, FIG. 33 illustrates an example of a method for reducing a loop of a nested pair of rigidizing members within a body lumen (e.g., reducing the curvature of a body lumen such as the colon) that modifies FIGS. 31 and 32 to include the initial pull-back step. In FIG. 33, the nested apparatus (e.g., the nested inner rigidizing member and the outer rigidizing member) may be navigated through a loop and the distal end region of the nested apparatus may extend at least to the descending color or beyond 3301. A loop of the nested apparatus may be detected 3303. To reduce the loop, both the inner rigidizing member and the outer rigidizing member may first be de-rigidized (e.g., transitioned to a relaxed configuration) 3305.

Thereafter, both the inner rigidizing member and the outer rigidizing member may be pulled back by pulling proximally on both of the inner and outer rigidizing members (withdrawing them) 3306. The inner and outer rigidizing members may be pulled from the proximal end (e.g., handle) regions of each. The steerable distal end of the inner (or in some examples, inner and outer) rigidizing members may be steered to maintain lumen-centricity while pulling the proximal end proximally. The distal end region position may be monitored. In examples in which the distal end region of the inner or inner and outer rigidizing members are not hooked or attached (e.g., by vacuum, etc.) to the lumen, when the distal end region begins to move proximally back while pulling, pulling proximally may be stopped and, if the loop is not fully reduced or minimized, either just the inner rigidizing member (as in FIG. 32) or both the inner rigidizing member and the outer rigidizing member (as in FIG. 31) may then be rotated, e.g., by rotation of the proximal handle regions of, to a rotational angle of between about 160-540 degrees 3307. In some examples, the shape of the inner rigidizing member and the outer rigidizing member (or both) may be monitored to confirm that the colon is moving out of the plane; if movement is not confirmed then the inner rigidizing member and the outer rigidizing member may be further rotated in the same direction until the loop starts to move 3309. In any of these examples, torque sensing may be used to provide feedback to assure that rotation of the inner rigidizing member and/or the outer rigidizing member remains within safe parameters. If the sensed torque, or if the rate of change of the sensed torque, exceeds a safety threshold then the rotation may be stopped. Once the loop moves out of the plane it was originally in (e.g., once the inner rigidizing member has been rotated together to between 160-540 degrees), the nested apparatus may be pulled back from the proximal end 3311. As before, during withdrawal of the proximal end of the nested apparatus, the steerable distal end region of either the inner rigidizing member and/or the outer rigidizing member may be continuously steered to maintain lumen-centricity of the distal end region. Optionally, if the distal end region begins to move proximally (e.g., starts withdrawing), then the inner rigidizing member and the outer rigidizing member may be further rotated, e.g., another 40-60 degrees and the withdrawal of the proximal end may be continued until the loop is sufficiently reduced 3313.

In some examples of the methods described herein the distal end region of the outer rigidizing member and/or the inner rigidizing member may be at least loosely anchored to the wall or region of the lumen (e.g., colon), e.g., by vacuum or forming a hook as described herein. A similar technique may be used in these examples, including optionally detecting the loop, attaching or least loosely anchoring the distal end region (e.g., the steerable distal end region may be hooked, a vacuum may be applied, an expandable member may be expanded, etc.). Thereafter, both the inner rigidizing member and the outer rigidizing member may be de-rigidized (e.g., made flexible). Note that in variations forming a hook region with the steerable distal end, the hook shape (formed by the steerable distal end region) may be maintained. The inner rigidizing member and the outer rigidizing member may then be withdrawn, as described.

### Torsional Stiffening

Any of the apparatuses including one or more rigidizing members described herein may be configured with one or more components to increase torsional stiffness and/or to compensate for low torsional stiffness. As described above, a rigidizing apparatus 100 (including a rigidizable member) may be formed of a plurality of layers that, in the un-rigidized configuration, may generally slide over each other. In particular, apparatuses including braid layers (formed of a braid or weave) may be configured to have a high degree of flexibility in the un-rigidized configuration and may therefore have a low braid angle, α, relative to the long axis of the member. For example, the braid angle may be less than about 40 degrees, less than about 35 degrees, between about 5-40 degrees, between about 5-35 degrees, between about 10-40 degrees, between about 15-40 degrees, between about 5 and less than 35 degrees, etc.

Thus, in the un-rigidized configuration the rigidizable member may have a low torsional stiffness and therefore not transmit torque particularly well along the length (e.g., from a proximal to a distal end) of the rigidizable member. Thus, it would be beneficial to provide one or more torque transmitting features (e.g., braids, laser cut hypotubes, etc.) that provide torsional stiffness in the un-rigidized configuration without substantially decreasing the flexibility of the un-rigidized member. In some examples a torque-transmitting element may be included that is separate from the rigidizing braid layer.

Any of these apparatuses may include a torque transmitting feature configured as an internal channel (such as, but not limited to a working channel wall or reinforcement). For example, a working channel may be configured as a torque transmitting channel and may be referred to herein as a torque transmitting working channel. A torque transmitting internal channel, such as a torque transmitting working channel, may be formed, e.g., of a laser-cut hypotube having a high torsional stiffness, but with a high flexibility in bending. A torque transmitting working channel may be configured so that the wall of the working channel is formed of torque transmitting feature (e.g., laser cut hypotube). This high torsional stiffness member may be coupled to the lower torsional stiffness wall(s), e.g., outer wall of the rigidizable member. For example, the high torsional stiffness member may be coupled at the bulkhead of the elongate rigidizable member, such as the inner layer 115.

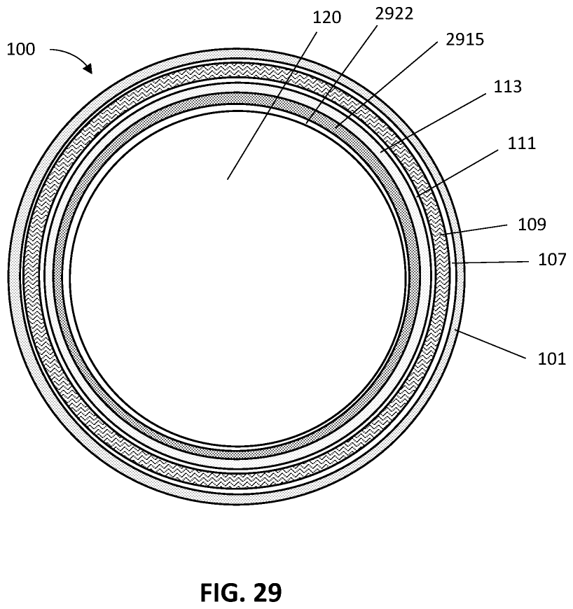

FIG. 29 illustrates one example of an apparatus in which the innermost layer 2915 or a layer adjacent to the innermost layer (e.g., radially outside of the innermost layer) is configured as a cut hypotube that has been cut to form a highly flexible member that also had a high transmission of torque. This configuration may be used either with the vacuum actuated configurations (as shown in FIGS. 3A-3F and 29) or in a positive-pressure actuated configuration (as shown in FIGS. 4A-4B). An additional sealing layer of material 2922 (e.g., polymeric material) that may maintain the pressure applied (either positive and/or negative pressure within the walls of the rigidizable member may be included. In FIG. 29, this sealing layer is shown one the inside surface of the high torsional stiffness layer 2915, but it may be positioned on the outer layer, adjacent to the slip layer 113. For example, the sealing layer 2922 may be laminated or sealed to the laser-cut hypotube 2915 without significantly decreasing the flexibility of the laser cut hypotube.

Alternatively or additionally, any of the apparatuses described herein may include a torque transmission feature that includes a braid having a high-angle relative to the long axis (e.g., an angle of greater than 35, greater than 40, greater than 45, etc.). that is configured to transmit torque. In some examples the torque transmission feature is configured to include wide, counter-wound wires that are constrained radially. This may be used, e.g., in a telescoping configuration in which an inner elongate rigidizable member and an outer elongate rigidizable member are included.

As described above, a high torsional stiffness member may be helpful when reducing, including as described above, automatically or semi-automatically reducing the curvature of a colon. Torsional stiffness may allow the apparatus to be rotate and flip over in a predictable and controllable manner. Torsional stiffness may also allow the user to rotationally orient the apparatus in the un-rigidized configuration. For example, any of these apparatuses may be configured to automatically level or orient the distal end of the elongate rigidizable member (e.g., to maintain the plane of the distal end, which may be curved or hooked, relative to the colon while moving the elongate rigidizable member). For example, the apparatus may include a control for executing a series of movements to level the elongate rigidizable member so that the distal end region of the elongate rigidizable member is oriented in a predefined manner relative to either the patient or an external reference frame (e.g., gravity). In some example the series of movements may be configured to rotate an inner elongate rigidizable member relative to the outer elongate rigidizable member in variations including two (or more) elongate rigidizable members, such as telescoping elongate rigidizable members. The apparatus may precisely rotate the inner elongate rigidizable member relative to the outer elongate rigidizable member (or vice versal), e.g., between 30-180 degrees. The apparatus may determine the reference frame based on one or more sensors, including the shape sensors, visual sensors (e.g., cameras) and/or an accelerometer or other gravimetric sensor, which may be, e.g., at a distal end region of the elongate rigidizable member. In some examples the apparatus may be configured to maintain the orientation (relative to a reference frame, such as relative to gravity) during operation of the apparatus by rotating the inner elongate rigidizable member relative to the outer elongate rigidizable member (or vice versal) when moving the apparatus within the body lumen including, but not limited to, when reducing the curvature of the body lumen (e.g., colon).

The torsional stiffness members, such as working channel torsional stiffness members described herein may allow decoupling of the bending flexibility of the apparatus from the torsional stiffness in rotation by including a separate torsional stiffness member, such as a cut (e.g., laser cut) hypotube forming the wall(s) of the working channel along the length of the rigidizable member. For example, a torsional stiffness member (e.g., laser-cut hypotube) forming a working channel of the apparatus may be coupled at the proximal end and at the distal end of the elongate rigidizable member. In some example the torsional stiffness member may be coupled distally to a distal end region that is bendable or steerable (e.g., by using one or more tendon-driven elements), such as coupled to the bulkhead portion of the elongate rigidizable member, e.g., the inner, e.g., supporting, layer(s). The proximal end region may be coupled to the handle region of the elongate rigidizable member. In examples including multiple elongate rigidizable members (e.g., inner and outer elongate rigidizable members), only one of the elongate rigidizable members (e.g., inner or outer) or some or all of the elongate rigidizable members may include a torsional stiffness member. For example, any of the apparatuses described herein may be configured to orient

the distal end region (including a tip of the elongate rigidizable member) at the target site by rotating the elongate rigidizable member. Thus, a camera and/or working channel of the elongate rigidizable member may be oriented by rotating the elongate rigidizable member that includes a torsional stiffness member within or part of the elongate rigidizable member, as described herein. This may enable the user to re-orient the clock position of the working channel and camera relative to a target structure (e.g., a polyp) or other reference frame (including but not limited to gravity). As mentioned above, the apparatus may be configured to drive rotation of the elongate rigidizable member in either the rigidized configuration or the un-rigidized configuration. For example an elongate rigidizable member of the apparatus may be rotated +/−180 degrees.

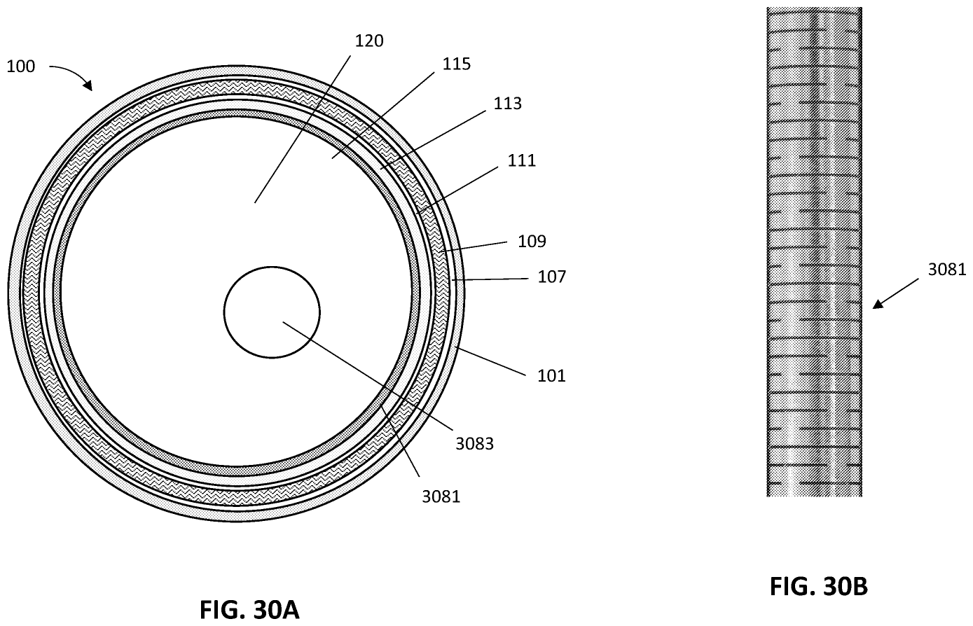

FIGS. 30A-30B illustrate and example of an elongate rigidizable member that includes a torsional stiffening member 3081 forming an internal working channel 3083. In this example the working channel is shown within the inner layer 115. In some examples the working channel is coupled to or formed within any of the other layers (or more than one of the layers), such as the gap layers 111, 107, the braid layer(s) 109, the slip layer 113, etc. Alternatively or additionally the torsional stiffening member may be coupled to an outer surface of the elongate rigidizable member 100, such as the outer layer 101.

FIG. 30B shows one example of a laser cut hypotube that may be used as part of the elongate rigidizable members described herein. Any appropriate pattern of the laser-cut hypotube may be bused, including a spiral pattern, alternating cuts, or the like. The space between the cuts (kerf) may be any appropriate size as well. The shape of the cuts of the hypotube may be selected to optimize the ease of bending (flexibility), while maintaining torsional stiffness. The hypotube may be formed of any appropriate material, including, for example, a stainless steel material a shape-memory material (e.g., a nickel titanium alloy such as a Nitinol, etc.).

As mentioned, any of these examples may replace a working channel with a laser cut hypotube in a positive pressure actuated configuration, such as when a double bladder may be used for rigidization. In general, apparatuses with a torque transmitting feature may permit rotation of the distal end of the elongate rigidizable member by rotating proximally, e.g., from a handle region. The torque transmitting feature may permit 1:1 correspondence between torque (rotation) of the proximal handle and rotation of the distal handle. In variations in which the laser-cut hypotube is used for the inner wall (e.g., innermost layer 115, 2115) or outer wall (e.g., outermost layer 101, 2101) the layer may be thin, but may maintain the strength resisting expansion and/or contraction of the hypotube-formed layer. In any of these configuration, including in particular when a laser-cut hypotube is used, the outer elongate rigidizing member may be stiff in torsion when rigidized and the handle may be locked in roll (e.g., to prevent rotation) to enhance fine rotation control of the inner rigidizing member. In some examples, unless the outer elongate rigidizing member is stiff in torsion when rigidized and the handle is locked in roll, the inner rigidizing member may not permit fine control of rotation of the inner rigidizing member.

As an alternative or in addition to the use of a torsional stiffening member forming an internal channel of an elongate rigidizable member, in some examples a torsional stiffening member may be inserted within the elongate rigidizable member and rotated to transmit torque to the elongate rigidizable member. For example a torsional stiffening member may be inserted into the lumen of the elongate rigidizable member and rotated to transmit torque to the distal end of the elongate rigidizable member. In any of these apparatuses a distal end region of the elongate rigidizable member may be configured to couple with (including releasably couple with) the inner torsional stiffening member. Thus, the torsional stiffening member may be inserted into the elongate rigidizable member after it has been positioned, including after the body lumen in which the elongate rigidizable member is inserted has been reduced.

For example, a torsional stiffening member (torque transmitting element) may be included as a torque cable or torque element (such as a laser-cut hypotube, braided catheter, speedometer cable, torque cable, shaft, etc.) that is connected to a core of the elongate rigidizable member and may be threaded along a working channels or working line of the apparatus. Any of these apparatuses may be configured so that the handle portion is permitted to float (e.g., to rotate freely) and/or to be driven in rotation. For example the handle region may include bearings to allow for free rotation, as during a reduction.

In some examples the torsional stiffening member may be configured to rotate within the elongate rigidizable member and may otherwise connect to the distal end region of the elongate rigidizable member through a bearing, bushing or sleeve to allow rotation of just the core without necessarily rotating the elongate rigidizable member. For example the torsional stiffening member may be coupled to the core and threaded along a working channel or line of the apparatus. This configuration may allow rotation of just a tip region (which may be rigidly coupled to the core torsional stiffening member and may require a lower torque to rotate. In some examples the distal end region of the apparatus may be configured to rotate.

Alternatively or additionally, in some of the apparatuses described herein including two or more telescoping elongate rigidizable members, one of the elongate rigidizable member (e.g., and inner elongate rigidizable member) may be configured to have a low transmission of torque, while the second elongate rigidizable member (e.g., an outer elongate rigidizable member) may have a higher torque transmission. The inner and outer elongate rigidizable members may be reversed (e.g., the inner elongate rigidizable member may be more torque transmissive while the outer elongate rigidizable member may be less torque transmissive). In some example torque generated by rotating the proximal end may result in 1:1 rotation of the distal end by rotating the higher torque transmitting elongate rigidizable member. For example, in some examples the inner elongate rigidizable member may include spline features that may couple to corresponding features on an inner diameter of the outer elongate rigidizable member.

In some of the apparatuses described herein, the apparatus may accommodate the low torque transmission by providing radially offset cameras and/or working channels around the perimeter of the distal end region of the elongate rigidizable member(s). For example, the apparatus may switch between multiple cameras arranged around the distal end region of the apparatus and/or by inserting a camera into different working channels arranged with exits at different radial positions around the distal end region of the elongate rigidizable member. For example, a camera can be swapped in either a “working channel” or a “camera channel” that are positioned at radial locations that are 180 degrees offset from each other. In some examples both a working channel and a camera channel may be roughly the same size; the camera may be slid out of the camera channel and re-inserted to another channel (e.g., working channel or another camera channel) depending on the location of a target tissue or region (e.g. polyp).

Alternatively or additionally, in some examples the apparatus may be configured as described above to articulate a distal bending section of the elongate rigidizable member to drive rotation (turning) of the elongate rigidizable member from the distal end region first, while allowing the more proximal end region to rotate freely. For example, any of these apparatuses may include a force and/or position sensor in the bending section(s) of the elongate rigidizable member (such as an inner or child elongate rigidizable member). The apparatus may also include a bearing at the handle region to allow free rotation, as described above.

In some of the apparatuses described herein the system may be configured to provide telescoping and independent rotation of an inner elongate rigidizable member relative to an outer elongate rigidizable member, as mentioned above. In some examples, the apparatus may be configured to rotate the body lumen (e.g., colon) around the apparatus by rotating the outer elongate rigidizable member after anchoring it to the wall of the body lumen. For example, a balloon on a distal end of the outer elongate rigidizable member may be used to rotate the lumen wall by rotating the elongate rigidizable member from the proximal end. This may be done in conjunction with or instead of rotating the inner elongate rigidizable member, which may be rotated in the opposite direction. This may allow the apparatus to re-orient the working and/or camera channel(s) relative to the tissue, particularly where these channels are located on an inner elongate rigidizable member. In some of these variations the outer elongate rigidizable member may be more torsionally stiff than the inner elongate rigidizable member.

As mentioned above any of the elongate rigidizable members described herein may be torsionally stiff in the rigidized confirmation. Thus in some examples the apparatus may be configured to partially rigidize the elongate rigidizable member, for example, along a proximal end region of the apparatus. This may reduce the length of the elongate rigidizable member that is less torsionally stiff. For example, the apparatus may include different rigidizable regions along the length of the apparatus. To perform a rotation of a distal end of the elongate rigidizable member, the apparatus may be rigidized up until a curved region starts (e.g., or up until the insertion of the apparatus within the body, such as within the anal sphincter). This may effectively shorten the length of the torsionally compliant system and allow for greater transmission of torque.

It should be understood that any feature described herein with respect to one embodiment can be combined with or substituted for any feature described herein with respect to another embodiment. For example, the various layers and/or features of the rigidizing devices described herein can be combined, substituted, and/or rearranged relative to other layers.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-90. (canceled)

91. A method of reducing curvature of a colon using a robotic system, the method comprising:
navigating an elongate member distally into the colon; and
activating a set of movements of the robotic system to control a steerable distal end region of the elongate member relative to a wall of the colon while withdrawing a proximal portion of the elongate member proximally out of the colon to reduce the curvature of the colon.

92. The method of claim 91, wherein activating the set of movements of the robotic system comprises adjusting a rigidity of the elongate member.

93. The method of claim 91, wherein activating the set of movements of the robotic system comprises activating a set of preprogrammed movements.

94. The method of claim 91, wherein the elongate member comprises a first elongate member and a second elongate member that is axially slideable within the first elongate member.

95. The method of claim 91, further wherein activating the set of movements of the robotic system comprises allowing at least one of a first elongate member and a second elongate member to freely rotate at a proximal end region of the elongate member that is outside of the colon.

96. The method of claim 91, wherein the elongate member comprises a first elongate member and a second elongate member, wherein the second elongate member is axially slideable relative to the first elongate member, further wherein activating the set of movements of the robotic system comprises rotating either or both the first elongate member and the second elongate member.

97. The method of claim 96, wherein rotating either or both the first elongate member and the second elongate member comprises rotating the first elongate member or the second elongate member in a direction in which the first elongate member or the second elongate member has a different bending stiffness in different rotational directions.

98. The method of claim 91, wherein activating the set of movements of the robotic system comprises withdrawing the proximal portion of the elongate member proximally out of the colon while maintaining a position or configuration of a distal end region of the elongate member.

99. The method of claim 91, wherein the elongate member comprises a first elongate member and a second elongate member that is axially slideable within the first elongate member, further wherein activating the set of movements of the robotic system comprises rigidizing, at least partially, ether the first elongate member or the second elongate member.

100. The method of claim 91, wherein activating the set of movements of the robotic system to control the steerable distal end region of the elongate member relative to the wall of the colon comprises forming a curve or hook at a distal end region of the elongate member while changing an articulation plane of the elongate member proximal to the distal end region.

101. The method of claim 91, wherein activating the set of movements of the robotic system to control the steerable distal end region of the elongate member relative to the wall of the colon comprises maintaining lumen-centricity of the steerable distal end region.

102. The method of claim 91, wherein activating the set of movements of the robotic system to control the steerable distal end region of the elongate member relative to the wall comprises securing the distal end region relative to the wall by one or more of: an inflatable balloon or suction.

103. The method of claim 91, wherein activating the set of movements of the robotic system to engage a wall of the colon comprises forming a pleat in the colon by pulling the wall of the colon proximally using the elongate member.

104. The method of claim 91, further comprising determining a shape of the elongate member within the colon.

105. The method of claim 91, further comprising detecting a loop in the colon prior to activating the set of movements.

106. The method of claim 91, wherein the elongate member comprises a first elongate member and a second elongate member that is axially slideable within the first elongate member, further wherein navigating an elongate member distally into the colon comprises alternately rigidizing and un-rigidizing the first elongate member and the second elongate member.

107. A method of reducing curvature of a colon using a robotic system, comprising:
inserting an elongate member into the colon, the elongate member comprising a first elongate rigidizable member and a second elongate member, wherein the second elongate member is axially slideable relative to the first elongate rigidizable member;
advancing the elongate member through a lumen of the colon; and
controlling the elongate member to reduce the curvature in the colon by maintaining a position, orientation or position and orientation of a distal end of the elongate member relative to the lumen of the colon and withdrawing a proximal end of the elongate member out of the colon.

108. The method of claim 107, further comprising detecting a loop in the colon.

109. The method of claim 107, wherein controlling the elongate member to reduce the curvature in the colon comprises automatically articulating the distal end of the first elongate rigidizable member or the second elongate member to maintain a hook or curve shape in a plane distal relative to the colon.

110. The method of claim 107, wherein controlling the elongate member to reduce the curvature in the colon further comprises maintaining lumen-centricity by steering the distal end of the first elongate rigidizable member or the second elongate member to maintain the distal end at or near a center of the colon.

111. The method of claim 107, wherein maintaining a position of a distal end of the elongate member comprises controlling the robotic system to inflate a balloon to anchor the first elongate rigidizable member or the second elongate member to the colon and/or to apply vacuum to anchor the first elongate rigidizable member or the second elongate member to the colon.

112. The method of claim 107, wherein advancing the elongate member through the colon comprises:
inserting the first elongate rigidizable member into the colon while the first elongate rigidizable member is in a flexible configuration;
supplying vacuum or pressure to the first elongate rigidizable member to transition the first elongate rigidizable member into a rigid configuration;
inserting a second elongate member, wherein the second elongate member is a rigidizable member, in a flexible configuration through the first elongate rigidizable member while the first elongate rigidizable member is in the rigid configuration such that the second elongate member takes on a shape of the first elongate rigidizable member in the rigid configuration; and
supplying vacuum or pressure to the second elongate member to transition the second elongate member from the flexible configuration to a rigid configuration.

113. The method of claim 122, further comprising controlling the robotic system to transition the first elongate rigidizable member and the second elongate member to the flexible configurations prior to reducing the curvature in the colon.

114. The method of claim 107, further comprising controlling the robotic system to drive rotation of the first elongate rigidizable member.

115. The method of claim 114, further comprising anchoring the second elongate member during rotation of the first elongate rigidizable member.

116. The method of claim 107, further comprising driving rotation of the second elongate member.

117. The method of claim 116, further comprising anchoring the first elongate rigidizable member during rotation of the second elongate member.

118. The method of claim 107, wherein maintaining the position of the distal end of the elongate member relative to the lumen of the colon comprises controlling the robotic system to deflect a distal end of the second elongate member such that it hooks onto a curve in the colon and further comprising the robotic system automatically rotating the distal end of the first or second elongate rigidizable member to maintain the distal end at or near a center of the colon.

119. The method of claim 107, wherein controlling the elongate member to reduce the curvature in the colon comprises controlling the robotic system to rotate a distal end of the second elongate member while the distal end is fixed to the colon while allowing at least one of the first elongate rigidizable member and the second elongate member to freely rotate at their proximal ends.

120. The method of claim 107, wherein controlling the elongate member to reduce the curvature in the colon comprises controlling the robotic system to rotate the first elongate rigidizable member in a first direction and the second elongate member in a second direction, opposite to the first direction.
